(19) United States
(12) Patent Application Publication
(10) Pub. No.: US 2010/0061951 A1
(43) Pub. Date: Mar. 11, 2010

(54) SELF-DISPERSED PIGMENTS AND METHODS FOR MAKING AND USING THE SAME

(75) Inventors: Puthalath K. Sujeeth, Ballwin, MO (US); John P. Kane, Ellisville, MO (US); Mark Ulrich, Florissant, MO (US); Daniel A. Ouellette, St. Peters, MO (US); Vincent Shing, Arnold, MO (US); Mihaela Madaras, Ballwin, MO (US); Carol D. Landi, Devon, PA (US); Alberto Guerrero, Succasunna, NJ (US)

Correspondence Address: MICHAEL BEST & FRIEDRICH LLP, 100 E WISCONSIN AVENUE, Suite 3300, MILWAUKEE, WI, 53202, US

(73) Assignee: SENSIENT COLORS INC., St. Louis, MO (US)

(21) Appl. No.: 12/545,790

(22) Filed: Aug. 21, 2009

Related U.S. Application Data
(63) Continuation-in-part of application No. 12/197,087, filed on Aug. 22, 2008.
(60) Provisional application No. 60/957,596, filed on Aug. 23, 2007.
(60) Provisional application No. 61/091,300, filed on Aug. 22, 2008.
(60) Provisional application No. 61/094,307, filed on Sep. 4, 2008.
(60) Provisional application No. 61/154,686, filed on Feb. 23, 2009.

(51) Int. Cl. A61K 8/84; C08G 73/02; C09D 179/02; A61Q 1/10

(52) U.S. Cl. 424/70.7; 528/422; 524/100

(57) ABSTRACT

A modified pigment that may include a polymer. The pigment may be directly attached to a nitrogen atom. The nitrogen atom may be attached directly or indirectly to a group that may include —S-Z. S may be a substituted or unsubstituted alkyl group, substituted or unsubstituted aromatic group, or polymer chain having a molecular weight range from about 300 to about 20000. Z may be a hydrogen, carboxyl, sulfonyl, phenolic, phosphoryl, ammonium, trimethylammonium, or tributylammonium group. Also, a modified pigment that may include a polymer. The pigment may be attached to an organic group through a carbon atom that is part of a N—C═N bond. A cosmetic formulation that may include a pigment covalently bonded to an organic group.

### CROSS-REFERENCE TO RELATED APPLICATIONS

This application is a continuation-in-part of and claims priority to U.S. patent application Ser. No. 12/197,087, filed Aug. 22, 2008, which claims priority under 35 U.S.C. §119(e) to U.S. Provisional Patent Application No. 60/957,596, filed Aug. 23, 2007. This application also claims priority under 35 U.S.C. §119(e) to U.S. Provisional Patent Application Nos. 60/957,596, 61/091,300, 61/094,307, and 61/154,686, filed Aug. 23, 2007, Aug. 22, 2008, Sep. 4, 2008, and Feb. 23, 2009, respectively. The entire contents of these applications are hereby incorporated by reference in their entireties.

### FIELD OF USE

The present invention relates to a method of making self-dispersing pigments. More particularly, the present invention relates to the surface modification of pigments. Pigments whose surfaces are modified through covalent bonding are known in the industry as self-dispersing pigments. The surface modifications may be carried out in an aqueous environment and may be eco friendly. The invention further relates to end use applications comprising surface-modified pigments, including, without limitation, coatings, paints, papers, adhesives, latexes, toners, textiles, fibers, plastics, inks, and cosmetic applications. Specific examples of end uses include, without limitation, printing ink for paper, textiles, fibers, metal deco and plastics, wood stains, writing instruments, color filters, and mascaras. The invention also relates to inks such as inkjet inks.

### BACKGROUND

Pigments offer several advantages over water-soluble dyes when it comes to inks, coatings, paints, papers, adhesives, latexes, toners, textiles, fibers, wood stains, color filters, plastics, and cosmetic applications. Pigments may exhibit at least one of greater lightfastness, waterfastness, optical density and edge acuity than water-soluble dyes. Unfortunately, pigments also have a greater propensity to settle during storage, thus initially limiting their use in demanding applications such as inkjet inks. The advent of media mills to grind pigment particles to sub-micron level combined with chemical additives for colloidal stability has propelled the use of pigment dispersions in inkjet ink formulations. However, chemical additives can increase the viscosity of dispersions such that it becomes difficult to jet the ink from the small orifices in an inkjet printhead. Moreover, chemical additives can add significant cost to the preparation of the materials listed above and are therefore economically unfavorable as well. Chemical additives, or dispersants, may not be bonded to the surface of the pigment and therefore, stabilization may be compromised. A need remains for improved ink compositions, especially for use in inkjet printers, which overcome at least some of the problems typically associated with current dye-based systems and pigment systems employing chemical additives. A need also remains for improved materials that use pigments, which overcome at least some of the problems typically associated with current dye based systems and pigment systems employing chemical additives.

### SUMMARY

In one aspect, the invention may provide a modified pigment that may include a polymer. The pigment may be directly attached to a nitrogen atom. The nitrogen atom may be attached directly or indirectly to a group that may include —S-Z. S may be a substituted or unsubstituted alkyl group, substituted or unsubstituted aromatic group, or polymer chain having a molecular weight range from about 300 to about 20000. Z may be a hydrogen, carboxyl, sulfonyl, phenolic, phosphoryl, ammonium, trimethylammonium, or tributylammonium group.

In another aspect, the invention may provide a modified pigment that may include a polymer. The pigment may be attached to an organic group through a carbon atom that is part of a N—C═N bond.

In yet another aspect, the invention may provide a cosmetic formulation that may include a pigment covalently bonded to an organic group.

In a further aspect, the invention may provide a method of modifying a pigment dispersion that may include reacting a substituted triazine with a pigment dispersion.

In another aspect, the invention may provide a method of modifying a pigment that may include reacting cyanuric chloride with a secondary compound or a mixture of secondary compounds to displace at least one reactive chlorine to form a substituted triazine and attaching the substituted triazine and at least one polymer to a surface of a pigment.

In a further aspect, the invention may provide a method of modifying a pigment that may include displacing at least one reactive chorine of a cyanuric chloride with at least one polymer to form a substituted triazine. The substituted triazine may be reacted with a pigment dispersed in a medium.

In yet another aspect, the invention may provide a method of modifying a pigment that may include attaching at least one polymer to a pigment in a pigment dispersion to form a modified pigment. The modified pigment may have directly attached a nitrogen atom. The nitrogen atom may be directly or indirectly attached to an organic group.

In a further aspect, the invention may provide a modified pigment that may include a pigment having attached a polymer and at least one of a group that may include N—S, a triazine substituted with at least one group that may include N—S, and a combination thereof. N may be a nucleophilic group and S may be an organic group.

### BRIEF DESCRIPTION OF THE DRAWINGS

FIG. 1 displays low resolution X-Ray Photoelectron Spectroscopy (XPS) spectra for untreated carbon black sample, Sensijet SDP 1000 carbon, and carbon black samples from Examples 25 thru 31.

FIG. 2 displays high resolution C1s XPS spectra for untreated carbon black sample, Sensijet SDP 1000 carbon and carbon black samples from Examples 25 thru 31.

FIG. 3 displays high resolution N1s XPS spectra for carbon black samples from Examples 25 thru 31.

FIG. 4 displays high resolution O1s XPS spectra for untreated carbon black sample, Sensijet SDP 1000 carbon and carbon black samples from Examples 25 thru 31.

FIG. 5 displays high resolution S2p XPS spectra for untreated carbon black sample, Sensijet SDP 1000 carbon, and carbon black samples from Examples 25 thru 31.

FIG. 6 displays high resolution Na1s XPS spectra for Sensijet SDP 1000 carbon and carbon black samples from Examples 25 thru 31.

FIG. 7 displays low resolution X-Ray Photoelectron Spectroscopy (XPS) spectra for untreated carbon black sample and carbon black samples from Examples 3, 8, 24 and 32.

FIG. 8 displays high resolution C1s XPS spectra for untreated carbon black sample and carbon black samples from Examples 3, 8, 24 and 32.

FIG. 9 displays high resolution N1s XPS spectra for carbon black samples from Examples 3, 8, 24 and 32.

FIG. 10 displays high resolution O1s XPS spectra for untreated carbon black sample and carbon black samples from Examples 3, 8, 24 and 32.

FIG. 11 displays high resolution S2p XPS spectra for untreated carbon black sample and carbon black samples from Examples 3, 8, 24 and 32.

FIG. 12 displays high resolution Na1s XPS spectra for carbon black samples from Examples 3, 8, 24 and 32.

FIG. 13 displays low resolution XPS spectra for untreated Pigment Blue 15 sample and Pigment Blue 15 samples from Examples 10, 14, and 21.

FIG. 14 displays high resolution C1s XPS spectra for untreated Pigment Blue 15 sample and Pigment Blue 15 samples from Examples 10, 14, and 21.

FIG. 15 displays high resolution N1s XPS spectra for untreated Pigment Blue 15 sample and Pigment Blue 15 samples from Examples 10, 14, and 21.

FIG. 16 displays high resolution O1s XPS spectra for untreated Pigment Blue 15 sample and Pigment Blue 15 samples from Examples 10, 14, and 21.

FIG. 17 displays high resolution S2p XPS spectra for untreated Pigment Blue 15 sample and Pigment Blue 15 samples from Examples 10, 14, and 21.

FIG. 18 displays high resolution Na1s XPS spectra for untreated Pigment Blue 15 samples and Pigment Blue 15 sample from Examples 10 and 21.

FIG. 19 displays high resolution Cu2p XPS spectra for untreated Pigment Blue 15 samples and Pigment Blue 15 sample from Examples 10, 14, and 21.

FIG. 20 displays low resolution XPS spectra for untreated Pigment Red No. 122 sample, Pigment Red No. 122 samples from Examples 17 and 22 and Pigment Violet No. 19 samples from Examples 6 and 7.

FIG. 21 displays high resolution C1s XPS spectra for untreated Pigment Red No. 122 samples and Pigment Red No. 122 samples from Examples 17 and 22 and Pigment Violet No. 19 samples from Examples 6 and 7.

FIG. 22 displays high resolution N1s XPS spectra for untreated Pigment Red No. 122 sample and Pigment Red No. 122 samples from Examples 17 and 22 and Pigment Violet No. 19 samples from Examples 6 and 7.

FIG. 23 displays high resolution O1s XPS spectra for untreated Pigment Red No. 122 sample and Pigment Red No. 122 samples from Examples 17 and 22 and Pigment Violet No. 19 samples from Examples 6 and 7.

FIG. 24 displays high resolution S2p XPS spectra for Pigment Red No. 122 sample from Example 22 and Pigment Violet No. 19 samples from Examples 6 and 7

FIG. 25 displays high resolution Na1s XPS spectra for Pigment Red No. 122 samples from Examples 17 and 22 and Pigment Violet No. 19 samples from Examples 6 and 7.

FIG. 26 displays low resolution XPS spectra for untreated Pigment Yellow No. 74 sample and for Pigment Yellow No. 74 samples from Examples 23 and 34.

FIG. 27 displays high resolution C1s XPS spectra for untreated Pigment Yellow No. 74 sample and for Pigment Yellow No. 74 samples from Examples 23 and 34.

FIG. 28 displays high resolution N1s XPS spectra for untreated Pigment Yellow No. 74 sample and for Pigment Yellow No. 74 samples from Examples 23 and 34.

FIG. 29 displays high resolution O1s XPS spectra for untreated Pigment Yellow No. 74 sample and for Pigment Yellow No. 74 samples from Examples 23 and 34.

FIG. 30 displays high resolution S2p XPS spectra for untreated Pigment Yellow No. 74 sample and for Pigment Yellow No. 74 samples from Examples 23 and 34.

FIG. 31 displays high resolution Na1s XPS spectra for untreated Pigment Yellow No. 74 sample and for Pigment Yellow No. 74 samples from Examples 23 and 34.

FIG. 32 displays low resolution XPS spectra for untreated Pigment Yellow No. 155 sample and for Pigment Yellow No. 155 samples from Examples 11 and 12.

FIG. 33 displays high resolution C1s XPS spectra for untreated Pigment Yellow No. 155 samples and for Pigment Yellow No. 155 samples from Examples 11 and 12.

FIG. 34 displays high resolution N1s XPS spectra for untreated Pigment Yellow No. 155 samples and for Pigment Yellow No. 155 samples from Examples 11 and 12.

FIG. 35 displays high resolution O1s XPS spectra for untreated Pigment Yellow No. 155 samples and for Pigment Yellow No. 155 samples from Examples 11 and 12.

FIG. 36 displays high resolution S2p XPS spectra for Pigment Yellow No. 155 samples from Examples 11 and 12.

FIG. 37 displays an in vivo test of a mascara prepared according to the present invention compared with control mascaras (see Example 50).

### DETAILED DESCRIPTION

Before any embodiments of the invention are explained in detail, it is to be understood that the invention is not limited in its application to the details of construction and the arrangement of components set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

In one aspect, the invention provides a method of modifying a pigment. The method may include attaching an organic group with charged end groups (negative or positive) through the intermediacy of a reactive molecule to produce a surface stabilized modified pigment. Without being limited by theory, it is believed that the stabilization is achieved by an even distribution of similarly charged groups which are covalently attached on sub micron sized pigment particles by the forces of repulsion.

In yet another aspect, the invention provides a dispersion that includes a self-dispersing pigment that has been formed by a reaction of a pigment with a reactive intermediate that has been attached to suitable organic molecules as described above. The selection of reactive intermediates that are stable in an aqueous environment is another aspect of the present invention.

In a further aspect, the invention provides a dispersion that includes a self-dispersing pigment comprising about 0.01 to about 1.0 mMoles of S and about 0.01 to about 2.0 mMoles of active hydrogen per gram of pigment, and water. In another aspect, the invention provides a dispersion that includes a self-dispersing pigment comprising about 0.06 to about 0.7 mMoles of S and about 0.07 to about 1.6 mMoles of active hydrogen per gram of pigment, and water.

In another aspect, the invention provides a polymer, polymeric resin, dispersant or binder attachment to a pigment or modified pigment, which enhances at least one durability property such as highlighter, water, or rub resistance while enhancing redispersibility. These properties are relevant to certain of the applications discussed herein, such as ink jet ink printing. Fast print speeds and small jet volumes (2-5 pico liters) also dictate low viscosity ink formulations particularly for thermal ink jetting. Attaching the polymer, polymeric resin, dispersant or binder reduces the quantity requirement. Additionally, the polymer stays with the pigment, and therefore, low viscosity formulations produce comparable results.

Method for Making Surface-Modified or Self-Dispersing Pigments

One aspect of the present invention relates to a method for making stable, self-dispersing pigments.

As used herein, the term “pigment” means a colorant insoluble in a solvent medium that is used to impart color to a substrate such as plain or coated paper, film and other types of receiving media. The pigment may also impart color to cosmetic formulations. Pigments may be black as well as other colors.

As used herein, the term “self-dispersing” pigment means a pigment having stabilizing groups covalently attached to its surface such that the pigment forms a stable aqueous dispersion in the absence of any additional dispersing agents.

As used herein, the term “stable” means that on aging the dispersion will undergo minimal changes as demonstrated by less than 10% change in measured critical properties (such as at least one of mean particle size, viscosity, surface tension and pH) when stored at ambient temperature over a period of at least about three months to six months to two years. Accelerated test methods include a heat stability test at about 70° C. for at least about one week or a heat stability test at about 70° C. for at least about four weeks.

As used herein, the term “redispersible” means that a modified pigment dispersion of the present invention may be dried to form a powder, and that powder may be redispersed in a medium. The medium may be an aqueous or non-aqueous medium. In the present invention, at least about 90% of the modified pigment present in the powder may be effectively redispersed in water as a dry powder. “Effectively redispersed” means that the particles may not be agglomerated in the end product.

As used herein, the term “attachment” may include direct or indirect attachment.

In one embodiment, the invention provides a method for making a surface modified or self-dispersing pigment comprising a pigment having attached at least one of a group comprising N—S, an X substituted with at least one group comprising N—S, and a combination thereof. For example, the pigment may have attached at least one of —X—N—S-Z, —N—S-Z and a combination thereof. In some embodiments, Z may have an ionic end group with a counter ion M. The pigment being modified may be dispersed in a medium. The modified pigment may have further attached a polymer. In another embodiment, the invention provides a method for making a surface modified or self-dispersing pigment comprising attaching at least one polymer to a pigment dispersed in a medium.

X may include, without limitation, a triazinyl group, with 1,3,5 triazine being preferred. N may be a nucleophilic group including, without limitation, an amine, an imine, pyridine, or thiol. S may include, without limitation, organic groups such as, a substituted or unsubstituted alkyl group, substituted or unsubstituted aryl group, substituted or unsubstituted aromatic group, or polymer chain having from about 1 to greater than 100 carbons or having a molecular weight range from about 300 to about 20000, suitably about 300 to about 8000. Z may be a hydrogen, carboxyl, sulfonyl, phenolic, phosphoryl, ammonium, trimethylammonium, or tributylammonium group. Z may have an ionic end group with a counter ion M. If present, M may be a halide, a negatively charged ion, ammonium, or a cation. In the case of stabilization by negative charge, ZM is an acidic tail group, wherein Z may be, without limitation, carboxyl, sulfonyl, phenolic, and phosphoryl and M may be either an ammonium or a cation. In the case of stabilization by positive charge, ZM may be a positively charged quaternary ammonium type tail group, wherein Z may be, without limitation, ammonium, trimethylammonium, and tributylammonium, and M may be a halide or any negatively charged ion. Examples of secondary compounds (N—S-ZM) include, without limitation, simple diamino aromatics or cationic polymers consisting of polyethyleneimines, polyguanidines, quaternary ammonium compounds etc.

In one embodiment, the method may comprise (1) optionally milling and dispersing a pigment to form an aqueous pigment dispersion (P)(R), the aqueous pigment dispersion (P)(R) comprising a) pigment (P), b) at least one of a polymer, a polymeric resin, a dispersant or binder (R) and c) water (alternatively, the aqueous pigment dispersion (P)(R) may be a commercially-available pigment dispersion such as those identified below and such as those stabilized with polymer additives); (2) reacting cyanuric chloride with about three equivalents of a secondary compound NSZ or a mixture of secondary compounds (NSZ, N₁S₁Z₁ and N₂S₂Z₂) to displace all reactive chlorines to form a substituted triazine; (3) reacting the substituted triazine with the aqueous pigment dispersion (P)(R) to form a self-dispersed pigment; and 4) optionally purifying the self-dispersed pigment to remove impurities, including the unattached dispersant. The resulting self-dispersed pigment may have attached at least one of a group comprising N—S, a triazine substituted with at least one group comprising N—S, and a combination thereof. For example, the modified pigment may have attached at least one of —X—N—S-Z, —X—N₁—S₁-Z₁, —X—N₂—S₂-Z₂, —N—S-Z, —N₁—S₁-Z₁, and —N₂—S₂-Z₂, depending on the secondary compounds or mixture of secondary compounds used. Optionally —X—N—S-Z, —X—N₁—S₁-Z₁, —X—N₂—S₂-Z₂, may be the same or different, just as —N—S-Z, —N₁—S₁-Z₁, —N₂—S₂-Z₂ may be the same or different. Additionally, each X may be substituted with —N—S-Z groups that are the same or different, and X may be substituted with (R) (such as an amine-containing (R)). (R) may attach directly to the pigment, may attach to the pigment indirectly via X, or a combination thereof. Indirectly via X means that additional groups may or may not be present between (R) and X.

In another embodiment, the method for making a surface modified or self-dispersing pigment may comprise (1) reacting cyanuric chloride with about three equivalents of a secondary compound NSZ or a mixture of secondary compounds (NSZ, N₁S₁Z₁ and N₂S₂Z₂) to displace all reactive chlorines to form a substituted triazine; (2) reacting the substituted triazine with a pigment (P), which may or may not be a pigment dispersion (e.g., a dispersion with or without (R)), to form a self-dispersed pigment; (3) optionally mixing at least one (R) with the self-dispersed pigment of step (2); (4) optionally purifying the self-dispersed pigment to remove impurities, including the unattached (R), if applicable. The resulting self-dispersed pigment may have attached at least one of a group comprising N—S, a triazine substituted with at least one group comprising N—S, and a combination thereof. For example, the modified pigment may have attached at least one of —X—N—S-Z, —X—N₁—S₁-Z₁, —X—N₂—S₂-Z₂, —N—S-Z, —N₁—S₁-Z₁, and —N₂—S₂-Z₂, depending on the secondary compounds or mixture of secondary compounds used. Optionally —X—N—S-Z, —X—N₁—S₁-Z₁, —X—N₂—S₂-Z₂, may be the same or different, just as —N—S-Z, —N₁—S₁-Z₁, —N₂—S₂-Z₂ may be the same or different, and X may be substituted with (R) (such as an amine-containing (R)). Additionally, each X may be substituted with —N—S-Z groups that are the same or different. (R) may attach directly to the pigment, may attach to the pigment indirectly via X, or a combination thereof. Indirectly via X means that additional groups may or may not be present between (R) and X. Alternatively, mixing at least one (R) may be conducted with step (1) prior to step (2). Alternatively, reacting the substituted triazine with a pigment (P) and mixing at least one (R) may be conducted concurrently or substantially concurrently.

During the substitution step, at least one chlorine of the cyanuric chloride is substituted with the secondary compound N—S-Z. This substitution may impart charge and bulk to the surface of the pigment. The substitution step may take place in an aqueous media. The choice of functional groups at the acidic tail is dictated by the final application while the functional groups at the basic head must have sufficient nucleophilicity to displace the chlorine in cyanuric chloride. The secondary compound may comprise polymers, amines, amino acids, alcohols, thiols, and combinations thereof. Examples of secondary compounds include, without limitation, amino benzoic acids, amino benzene sulfonic acids, amino phenols, amino sulfonic acids, polyethoxylated amino acids, sodium sulfanilate, sulfanilic acid, sodium p-aminobenzoate, p-aminophenol, ethyl 4-aminobenzoate, taurine, oleic acid (amino), sodium aminooleate, tetramethylammonium 4-aminobenzoate, and sodium 4-aminophenolate. Additional secondary compounds include organic polymeric substrates. Examples of organic polymeric substrates may include, without limitation, pentaethylenehexamine, linear alkyl and branched ethoxy and propoxy chain polymers with a known molecular weight range of 300-3000 MW, available from Huntsman Chemicals under the trade name “Surfonamines,” linear polyethoxy polymeric amines, linear propoxy polymeric amines, and polyethyleneimines sold under the trade name “Epomines.” Specific examples of organic polymeric substrates may include, without limitation, Surfonamine B30, L100, L300, B60, and L207 (Huntsman), pentaethylene hexamine (Akzo Nobel), and Epomin SP-012 (Nippon Shokubai).

As set forth above, R may be a polymer, a polymeric resin, a dispersant or binder. In one embodiment, a dispersant may be a polymer with functional groups that may be activated to form a radical and attach to the surface of the pigments. R may already present in a raw pigment dispersion; may be added to a raw pigment; added to a raw pigment dispersion; or combinations thereof. Specific examples of polymers include, but are not limited to polystyrene-co-maleicanhydride resins (SMA), poly(styrene-co-maleic anhydride) cumene terminated resins, PEI, PEHA, styrene-acrylic (SA), pentaethylenehexamine, linear alkyl and branched ethoxy and propoxy chain polymers with a known molecular weight range of 300-3000 MW, available from Huntsman Chemicals under the trade name “Surfonamines,” linear polyethoxy polymeric amines, linear propoxy polymeric amines, styrene acrylic copolymers available from BASF under the trade name “Joncryls,” and polyethyleneimines sold under the trade name “Epomines,” etc. Specific examples of polymers may include, without limitation, Joncryl HPD 96, HPD 296, HPD 196 (BASF), Surfonamine B30, L100, L300, B60, and L207 (Huntsman), poly(styrene-co-maleic anhydride) cumene terminated resin (molecular weights of 1700 and 1900) (Aldrich), pentaethylene hexamine (Akzo Nobel), and Epomin SP-012 (Nippon Shokubai). R may attach directly to the pigment, to the pigment via at least one of X—N—S-Z, or a combination thereof.

Examples of commercial aqueous pigment dispersions (P)(R) that may be used in the present invention, include, but are not limited to, Sensijet® Magenta PV19, Sensijet® Cyan PB15:3, Sensijet® Yellow PY155, and Sensijet® Black PB 094. Additional examples of commercial aqueous pigment dispersions (P)(R) include, but are not limited to, Sensijet® Black SDP pigment dispersions (100, 1000 and 2000) available from Sensient Colors Inc. Examples of pigment dispersions (under the trade name Lemantex) available from Sensient Imaging Technologies—Specialty Inks and Colors (Switzerland) include, but are not limited to, Cyan PB 15:3, Blue PB 60, Green PG 7, Magenta PR 122, Red PR 254, Orange PY 83, and Yellow PY 120. Additional examples of pigment dispersions that may be modified using the methods of the present invention may be found in U.S. Pat. No. 4,597,794 issued Jul. 1, 1986, U.S. Pat. No. 5,172,133, issued Dec. 15, 1992, and U.S. Pat. No. 4,156,616, issued May 29, 1979, each of which is hereby incorporated by reference.

To help illustrate the invention, a specific example of the first embodiment is provided below, wherein P represents a pigment and R represents a polymer, polymeric resin or dispersant.

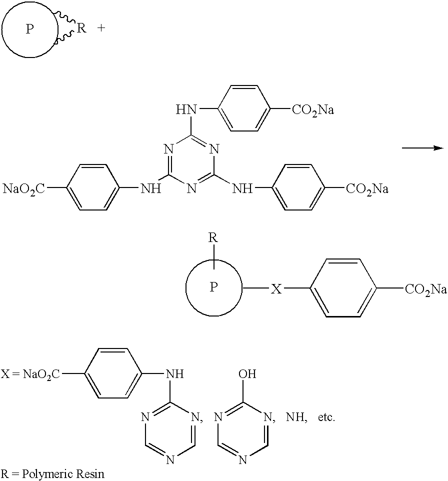

In some embodiments, the modified pigment may comprise a polymer, and the pigment may be directly attached to a nitrogen atom, the nitrogen atom being attached directly or indirectly to an organic group. The organic group may comprise —S-Z. The nitrogen atom may be part of an amino group. The nitrogen atom may be part of an aromatic amino acid such as, without limitation, aminobenzoic acid, aminobenzenesulfonic acid, or aminophenol. The pigment may be covalently bonded to an aromatic substrate with an amine and quarternary ammonium end groups through an amine nitrogen. In other embodiments, the modified pigment may comprise a polymer, and the pigment may be attached to an organic group through a carbon atom that is part of a N—C═N bond. The N—C═N bond may be part of a triazine.

More generally speaking, surface modified pigments may be formed by milling raw pigments to a fine grind (typically less than 100 nm) and subsequently attaching small organic molecules as stabilizing groups. Surface modification chemistries, including those described herein, may also be used on raw pigment dispersions that comprise raw pigment, a dispersant, and water. A raw pigment may be dispersed as known in the art using a dispersant to form a raw pigment dispersion The raw pigment dispersion, rather than the raw pigment (e.g., in powder form), may be used in the surface modification techniques described herein, as well as other surface modification techniques that are well known in the art. In addition, raw pigment dispersions and raw pigment may be used together as starting materials in the surface modification techniques described herein. Any combination of raw pigments, raw pigment dispersions, surface modified pigments, and surface modified pigments from raw pigment dispersions, may be used as starting materials in the surface modification techniques described herein. Examples of other surface modification techniques that may be used with this technique include, but are not limited to, U.S. Pat. Nos. 5,085,698, 5,310,778, 5,172,133, 4,156,616, 4,597,794B1, and 6,406,143B1, each of which is incorporated herein by reference.

In using any of the previous surface modification chemistries to modify a raw pigment dispersion, the dispersant in the raw pigment dispersion, as well as the compounds described above, may attach to the surface of the raw pigment during the surface modification. In this way, dispersants capable of forming radicals and substituted reactive intermediates may be attached to the surface of the pigment simultaneously. This may form a stable pigment dispersion. Any remaining dispersant that does not attach to the surface of the pigment during the surface modification process, i.e., any dispersant that is only adsorbed by the pigment, and not attached, may be removed through a purification process.

In one embodiment, the commercial pigment dispersion may be modified without any milling required. If smaller particles are desired, then the dispersion may be milled prior to or at any point during the attachment process. For example, a Buhler micro media mill may be used. In a further embodiment, the dispersant may be added to a raw pigment and the pigment and dispersant may then be milled prior to or at any point during the attachment process. In yet a further embodiment, the dispersant may be added to a raw pigment and the pigment and dispersant may then be milled, or a raw pigment dispersion may be milled, and before or at any point during milling, additional polymer or substituted reactive intermediate may be added. A grind aid may also be milled with the raw pigment and dispersant. The amount of dispersant added may be controlled to affect the final amount of dispersant attached to the surface of the pigment. Milling performed prior to chemical treatment may allow the use of common mill chambers and parts while preventing re-agglomeration during the attachment process.

Pigments

Pigments that may be surface modified according to the present invention may include, but are not limited to, azo pigment, phthalocyanine pigment, anthraquinone pigment, quinacridone pigment, thioindigo pigment, triphenylmethane lake pigment, and oxazine lake pigment. Specifically, those having yellow colors include, for example, C. I. Pigment Yellow 1, 2, 3, 4, 5, 6, 10, 12, 13, 14, 16, 17, 65, 74, 83, 97, 120, 138, 150, 151 and 155. Those having red colors include, for example, C. I. Pigment Red 1, 2, 3, 4, 5, 6, 7, 8, 9, 10, 11, 12, 13, 14, 15, 16, 17, 18, 21, 22, 23, 31, 32, 37, 38, 41, 48, 49, 50, 51, 52, 57, 58, 60, 64, 83, 88, 89, 90, 112, 114, 122, 123, 166, 188, 202, 254, C. I. Pigment Violet 19 and 23. Those having blue colors include, for example, C. I. Pigment Blue 1, 2, 15, 15:3, 15:4, 16, 25, 60, and 75. Those having green colors include, for example C.I. Pigment Green 7 and 36. Those having black colors include, for example, C. I. Pigment Black 1 and 7. Commercially available colored pigments include, for example, Pigment Red 122 and Pigment Violet 19 available from Lansco Colors, Montvale, N.J. or BASF Color, Charlotte, N.C. or Clariant Colors, Charlotte, N.C. or Sun Chemical, Cincinnati, Ohio, Pigment Blue 15:3, Pigment 15:4, Pigment Yellow 74 and Pigment Yellow 97 (available from BASF Color, Charlotte, N.C. or Clariant Colors, Charlotte, N.C. or Sun Chemical, Cincinnati, Ohio).

Suitable pigments also include carbon black. Carbon black is the generic name for carbon particles derived from the thermal decomposition or the incomplete combustion of natural gas and hydrocarbons, such as aromatic oils on coal tar basis, mineral oils, coal tar distillate, and acetylene. More than 100 individual grades of carbon black are available on the market today, each with its own distinctive set of characteristics and properties. Any acidic carbon black, neutral carbon black and alkaline carbon black may be beneficially subjected to the treatment disclosed in the present invention. This includes channel blacks, gas blacks, lamp blacks, thermal blacks, acetylene blacks and furnace blacks. More particularly, suitable carbon blacks include channel blacks. The quality of carbon black utilized will have an impact on the critical properties of the dispersion such as mean particle size, opacity, color shade, stability, etc. Examples of commercially available carbon blacks include, but are not limited to, those available from Cabot (Elftex 8, Black Pearls® 490, Black Pearls® 120, Monarch® 120, Monarch® 700, Monarch® 880, Monarch® 1000, Monarch® 1100, Monarch® 1300, Monarch® 1400, Mogul® L, Regal® 99R, Regal® 250R, Regal® 300R, Regal® 330R, Regal® 400R, Regal® 500R, Regal® 660R), Degussa (NIPex® 150 IQ, NIPex® 150, Printex® 55, Printex® 80, Printex® 90, Printex® A, Printex® G, Printex® U, Printex® V, Printex® 140U, Printex® 140V, Purex® LS 35, Corax® HP 160, Thermal Black N 990, NIPex® 160 IQ, NIPex® 90, Special black 4, Special black 4A, Special black 5, Special black 6, Special black 100, Special black 250, Color black FW1, Color black FW2, Color black FW2V, Color black FW18, Color black FW200, Color black S150, Color black S160 and Color black S170), Columbian (Raven® 780, Raven® 5000 UII, Raven®1255, Raven® 2500 U, Raven® 3600 U, Raven® 3500, Raven® 7000, Raven® 1220 and Raven® 1225) and Mitsubishi Kagaku K.K. (MA8, MA11, MA77, MA100, MA220, MA230, MA600, MCF88, #10B, #20B, #30, #33, #40, #44, #45, #45L, #50, #55, #95, #260, #900, 970#, #1000, #2200B, #2300, #2350, #2400B, #2650, #2700, #4000B and CF9).

Other pigments that may be surface modified according to the present invention may include, without limitation, pigments that have been FDA approved. These may be suitable for cosmetic applications. Acceptable pigments that may be used in cosmetics may be found in 21 C.F.R. §§70-82, which are hereby incorporated by reference. Specific examples of black pigments (carbon blacks), include, without limitation, high purity carbon black prepared by the oil furnace process, D&C Black No. 2, and Unipure Black LC 902 (available from Sensient Cosmetic Technologies).

Other pigments that may be surface modified according to the present invention may include, pigments that have been previously oxidized, sulfonated, or a combination thereof. Oxidants include, without limitation, nitric acid, ozone, hydrogen peroxide, persulfate, hypohalite, or a combination thereof. Aqueous oxidation of carbon black using sodium hypochlorite is taught by U.S. Pat. No. 2,439,442 issued Apr. 13, 1948 and U.S. Pat. No. 3,347,632 issued Oct. 17, 1967, each of which is hereby incorporated by reference. Hydrophilic groups comprising sulfonic acid are attached to pigments by sulfonation with sulfuric acid, oleum (fuming sulfuric acid) or a combination thereof. Attachment of sulfonic acid groups directly on the surface of the pigment may also be achieved by sulfonating with other known chemical agents such as chlorosulfonic acid or by displacement of a leaving group attached to the pigment, such as halogen with a suitable reagent such as sodium bisulfite. Following the oxidation, sulfonation, or combination thereof of the pigment, the surface of the pigment may be treated using the methods of the current invention. The oxidized or sulfonated pigment may be dispersed in a dispersion prior to treating it with the methods of the current invention. Examples of commercially available surface modified pigment dispersions include, without limitation, Sensijet® Black SDP 1000 carbon black dispersion and Sensijet® SDP 2000 dispersions (available from Sensient Colors Inc., St. Louis, Mo.). Other commercially available pigment dispersions available from Cabot Corporation as Cab-O-Jet self dispersed pigments and from Orient Chemicals as Bonjet black dispersions, may also be similarly modified.

Pigments are available in a variety of particle sizes. Generally, smaller particle sizes are associated with larger surface areas, and larger surface areas can accommodate a higher concentration of hydrophilic surface groups, which ultimately enhance the dispersibility of the pigment in aqueous-based media. Therefore, particle size can influence the dispersibility of a surface-modified pigment. For example, the average primary particle size of carbon blacks in the present invention may be less than about 50 nm, particularly less than about 30 nm, particularly less than about 20 nm, and more particularly less than about 10 nm. Aggregates of carbon black particles may be less than about 200 nm, particularly less than about 150 nm, and more particularly less than about 100 nm. The surface area of carbon black particles may be greater than about 100 m²/g, particularly greater than about 150 m²/g, and more particularly greater than about 200 m²/g. Pigment particles with larger dimensions may be comminuted to a desired size either before or during surface modification using any number of techniques known to those skilled in the art. Such techniques may include, but are not limited to, a ball mill, an attritor, a flow jet mixer, an impeller mill, a colloidal mill and a sand mill (e.g., one commercially sold under the trade name ‘Super Mill’, ‘Agitator Mill’, ‘Dyno-mill’ or ‘Beads Mill’). Mill media may include, but are not limited to, glass beads, zirconia beads, plastic beads and stainless steal beads. Mill media may comprise particles ranging in size from about 0.01 mm to about 5 mm, suitably from about 0.1 mm to about 3 mm. If the pigment is easily crumbled, a rotary homogenizer or an ultrasonic homogenizer may be used to reduce particle size. In one embodiment, a surface-modified black pigment is made from a commercial grade carbon black pigment consisting of primary particle sizes less than about 30 nm and aggregates not more than about 200 nm with a surface area greater than about 100 m²/g.

In some instances, prior to the creation of the self-dispersing pigments, the pigment may be wetted and milled to nano sized particles and dispersed using a grind-aid and/or a polymeric resin. The pigment may be in powder or wet cake form prior to milling with the aid of a grind aid. The milling may take place prior to, at any point during, or after the reaction with the substituted reactive intermediate or additional polymer. After the attachment reaction is complete, unattached grind-aid/resin may be removed using purification methods that are known to those skilled in the art, forming a dispersion containing primarily the modified pigment with attached substrates and water. Examples of grind aids include, but are not limited to Triton X-100 (available from Ashland Inc., Dublin, Ohio), Igepal CA-630 (available from Rhodia, Cranbury, N.J.), Surfynol CT 121, 131, and 141 (available from Air Products, Allentown, Pa.), and Lemantex Binder (available from Sensient Imaging Technologies S.A., Switzerland).

In such instances, a radical initiator such as a persulfate moiety is used to disproportionate and facilitate the attachment process. In some embodiments, the reaction may be carried out at a temperature of about 25° C. to about 90° C. The pigment may be milled to less than about 100 nm before, during, or after reacting the pigment with the substituted triazine. A defoamer may be added as needed to control foaming. Dye solutions and/or surfactants may be used as needed for wetting the pigment.

In embodiments where there are two slurries with different secondary compounds, the pigment is mixed with the slurries sequentially. The temperature of the dispersion may be maintained at about 0° C. to about 15° C. for a period of about 1 hour to about 2 hours. The mixture of the reactive compound (e.g., substituted triazine) dispersion and the pigment is then heated to elevated temperatures for a period of up to about 2 days. A free radical initiator such as potassium persulfate may be added to promote the reaction. The reaction temperature may be at least about 40° C., particularly at least about 50° C., and more particularly at least about 60° C. Furthermore, the reaction temperature may be less than or equal to about 90° C., particularly less than or equal to about 80° C., and more particularly less than or equal to about 60° C. This includes embodiments where the reaction temperature is about 50° C. to about 60° C., more particularly no more than 90° C. Generally, temperatures above 50° C. are required for the free radical initiator to be effective. This includes embodiments where the reaction time is from about 16 hours to about 24 hours. The contents of the reaction vessel are stirred during the reaction to insure adequate mixing. The modified pigment may be filtered to remove excess reactants and impurities.

In one embodiment, the reactive compound (such as cyanuric chloride) is reacted with the secondary compound in an acidic pH (about 2 to about 5) range. The acidic pH range increases the stability of the reactive compound and decreases the degree of undesirable reactions such as hydrolysis and self-condensation. The reactive compound reacts preferentially with a base such as a primary amine even when an amino phenol is used as the organic group. The reaction can be directed primarily to the amino end by the proper choice of the reaction conditions such as pH, temperature, and dilution which is well known to those skilled in the art. For example, the pH may be from about 2 to about 5 and the temperature may be from about 0° C. to about 5° C.

Optionally, while reacting the pigment with the groups described above (such as, for example, X, X—N—S-Z, N—S-Z, and/or (R)), the particle size of the pigment can be reduced by performing the reaction in a bead mill. Due to the corrosivity of the secondary compound, proper materials of construction resistant to strong acids and bases may be selected to prevent metal leaching into the product.

Reaction of the pigments with reactive compounds or secondary groups that include acid derivatives may create acidic surface groups that can lower the pH of the reaction mixture. A decrease in pH may result in a destabilization of the modified pigment dispersion or slurry of reactive compound and secondary compound during the substitution and may also result in an increase in viscosity. Therefore, the pH may be adjusted, as needed, before and during the substitution with a basic reagent. The pH of the reaction mixture during substitution may be greater than or equal to about 7, particularly greater than or equal to about 8, and more particularly greater than or equal to about 9. The pH may be adjusted by any known method in the art including, for example, the addition of base. Suitable bases may include, but are not limited to, alkali hydroxides and calcium free alkali hydroxides (e.g., NaOH, KOH, LiOH, NH₄OH), alkali carbonates and bicarbonates (e.g., NaHCO₃, KHCO₃), and organic bases (e.g., dimethylethanol amine and triethanol amine). In particular, a suitable pH adjuster comprises calcium free sodium hydroxide.

Surface Modified Pigment

After the reactions described above are complete, the self-dispersing pigment may be isolated from the reaction mixture as a dry powder. The resultant modified pigment may be purified by using any number of techniques known to those skilled in the art to remove unreacted raw materials, byproduct salts and other reaction impurities. Purification techniques may include, but are not limited to, filtration, centrifugation, or a combination of the two. The modified pigment may also be isolated, for example, by evaporation or it may be recovered by filtration and drying using techniques known to those skilled in the art.

Alternatively, the self-dispersing pigment may be delivered as concentrated aqueous pigment dispersion. Dispersions of the self-dispersing pigments of the present invention may be purified to remove organic and inorganic impurities and other undesirable free species which can co-exist in the dispersion as a result of the manufacturing process. Purification techniques may include, but are not limited to, water washing, reverse osmosis, and ultrafiltration. In some embodiments, dissolved impurities may be removed by ultrafiltration until the chloride and sulfate content of the feed sample adjusted to 10% solids is less than about 150 ppm, particularly less than about 100 ppm, and more particularly less than about 25 ppm. If necessary, the pH of the dispersion may be adjusted prior to purification. A sufficient amount of acid or base may be added to adjust the pH of the dispersion to at least about 7, particularly to at least about 8, and more particularly to at least about 9. This includes embodiments where the pH of the dispersion is about 7 to about 9. The dispersion may be concentrated if desired by removal of some of the water. In some embodiments, the dispersion is concentrated to at least about 8% solids, in others to at least about 14% solids, and in yet others to at least about 20% solids. This includes embodiments where the dispersion is concentrated to about 8% to about 16% solids. In other embodiments, the dispersion is concentrated to at least about 10% solids, in others to at least about 18% solids, and in yet others to at least about 20% solids. This includes embodiments where the dispersion is concentrated to about 10% to about 17% solids.

In some embodiments, the dispersion may be dried and reconstituted to at least about 40% solids. In other embodiments, the dispersion may be dried and reconstituted to about 60% to about 70% solids.

In some embodiments, pigments modified according to the present invention may be dispersed with unmodified pigment to form a dispersion comprising both modified and unmodified pigment.

A biocide may also be added to the dispersion to inhibit the growth of microorganisms. Examples of suitable biocides include, but are not limited to, sodium benzoate, pentachlorophenol sodium, 2-pyridinethiol-1-oxide sodium, sodium sorbate, sodium dehydroacetate, benzisothiazolinone, 1,2-dibenzothiazolin-3-one, methylisothiazolinone and chloromethylisothiazolinone. Commercially available biocides include Proxel® CRL, Proxel®BDN, Proxel® GXL, Proxel® XL-2, and Proxel® TN (available from Arch Chemicals, Smyrna, Ga.) and XBINX® (available from PMC Specialties Group, Inc., Cincinnati, Ohio). Typically, a small amount, such as 0.05 to 5%, particularly 0.1 to 1%, and more particularly 0.2 to 0.4% by weight of biocide, is used in the dispersion. This includes 0.3% by weight biocide.

The dispersion may be filtered through filter cartridges as required for the designated end use of the dispersion. In some embodiments, the nominal pore size of the filter cartridge is less than or equal to about 5 microns, particularly less than or equal to about 1 micron, particularly less than or equal to about 0.5 micron, and more particularly less than or equal to about 0.2 micron.

In addition to powders and dispersions, the self-dispersing pigment may also be isolated as a water wet presscake. In presscake form, the self-dispersing pigment is not agglomerated to the extent that it is in dry form and thus the self-dispersing pigment does not require as much deagglomeration when used, for example, in the preparation of inks.

If desired, the charge-balancing counterions associated with the surface-modifying groups as a result of the attachment/substitution process may be at least partially substituted or changed with the use of suitable base or salt form or exchanged or substituted with other suitable cations using known ion-exchange techniques such as ultrafiltration, reverse osmosis, conversion to acid form as an intermediate and the like. Examples of counterions include, but are not limited to, alkali metal ions (e.g., Na⁺, K⁺ and Li⁺), NR₁R₂R₃H⁺, and combinations thereof, wherein R₁, R₂ and R₃ may independently be H or C₁-C₅ alkyl groups that may be unsubstituted or substituted (e.g., tetraethylammonium ion (TEA), tetramethylammonium ion (TMA), ethanolammonium ion, triethanolammonium ion, tetrabutylammonium ion, etc).

Properties of Modified Pigments

The self-dispersing pigments may exhibit at least one of long-term and high temperature stability, higher water and highlighter fastness than expected of a pigment particle with attached sulfonic or carboxylic acid groups, and have a particle size distribution suitable for use in high speed jetting applications.

The self-dispersing pigments may possess the following properties. The % of solids in the modified pigments may be from about 5-30, suitably about 10-30, suitably about 10-22.

The pH of the modified pigment dispersion may be from about 5 to about 12, suitably about 5 to about 10.

The viscosity of the modified pigment dispersion may be from about 1 to about 11 cps, particularly about 2 to about 8 cps.

The surface tension of the modified pigment dispersion may be from about 30 to about 72 dynes/cm, suitably about 30 to about 60 dynes/cm.

The amount of Na and K in the modified pigment dispersion may be a measure of a newly attached anionic substrate (sulfanilic or 4-aminobenzoic acid or 4-aminophenol as Na/K forms). The amount of Na may be from about 100 to about 7500 ppm and the amount of K may be from about 30 to about 3000 ppm, suitably about 30 to about 2500 ppm.

The increase in the sulfur content in the modified pigment dispersion may be due to the introduction of a sulfonyl group and/or attachment of a sulfonated substrate such as, without limitation, sulfanilic acid. The amount of sulfur in the modified pigments may be from about 0 ppm to about 3000 ppm, suitably about 50 ppm to about 3000 ppm. In one embodiment, the amount of sulfur in the modified pigments may be about 50 ppm for 4-aminobenzoic acid and 4-aminophenol attachments. In another embodiment, the amount of sulfur in the modified pigments may be about 2000 ppm when a sulfanilic acid is attached to the pigment.

Carbon black modified according to the present invention may comprise about 0.3 to about 1.7 mMoles, suitably about 0.403 to about 1.584 mMoles of active hydrogen per gram of pigment.

Cyan pigments modified according to the present invention may comprise about 0 to 1 mMoles, suitably 0.03 to about 0.3 mMoles, suitably about 0.050 to about 0.112 mMoles of sulfur per gram of pigment. Cyan pigments modified according to the present invention may comprise about 0.2 to about 0.9 mMoles, suitably about 0.395 to about 0.732 mMoles of active hydrogen per gram of pigment.

Magenta pigments modified according to the present invention may comprise about 0 to 1 mMoles, suitably 0.02 to about 0.2 mMoles, suitably about 0.034 to about 0.140 mMoles of sulfur per gram of pigment. Magenta pigments modified according to the present invention may comprise about 0.1 to about 1.2 mMoles, suitably about 0.196 to about 0.911 mMoles of active hydrogen per gram of pigment.

Yellow pigments modified according to the present invention may comprise about 0 to about 1 mMoles, suitably about 0.02 to about 1.0 mMoles, suitably about 0.065 to about 0.081 mMoles, suitably about 0.034 to about 0.075 mMoles of sulfur per gram of pigment. Yellow pigments modified according to the present invention may comprise about 0.1 to about 1.0 mMoles, suitably about 0.196 to about 0.757 mMoles, suitably about 0.148 to about 0.442 mMoles of active hydrogen per gram of pigment.

Violet pigments modified according to the present invention may comprise about 0 to 1 mMoles, suitably 0.03 to 0.3, suitably about 0.022 to about 0.087 mMoles of sulfur per gram of pigment. Violet pigments modified according to the present invention may comprise about 0.2 to about 0.4 mMoles, suitably about 0.283 to about 0.347 mMoles of active hydrogen per gram of pigment.

Pigments modified according to the present invention may be redispersible in an aqueous or non-aqueous medium.

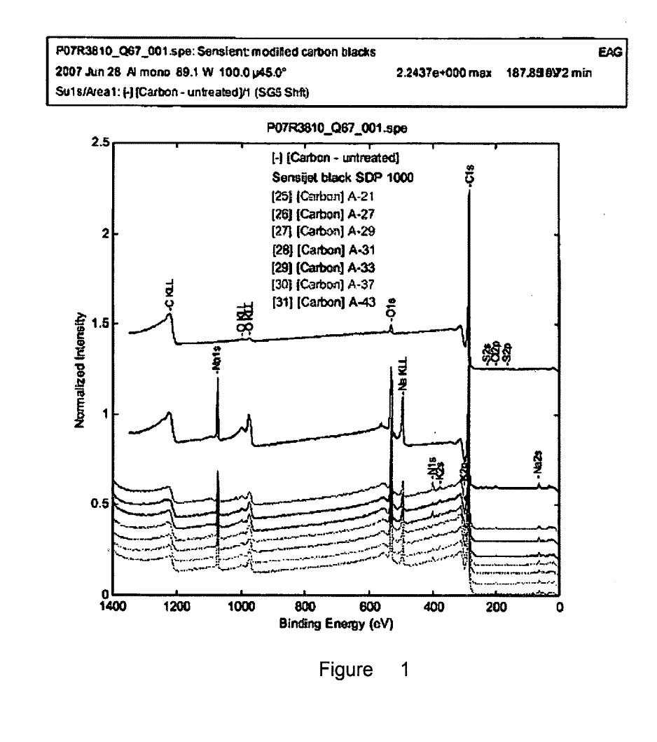

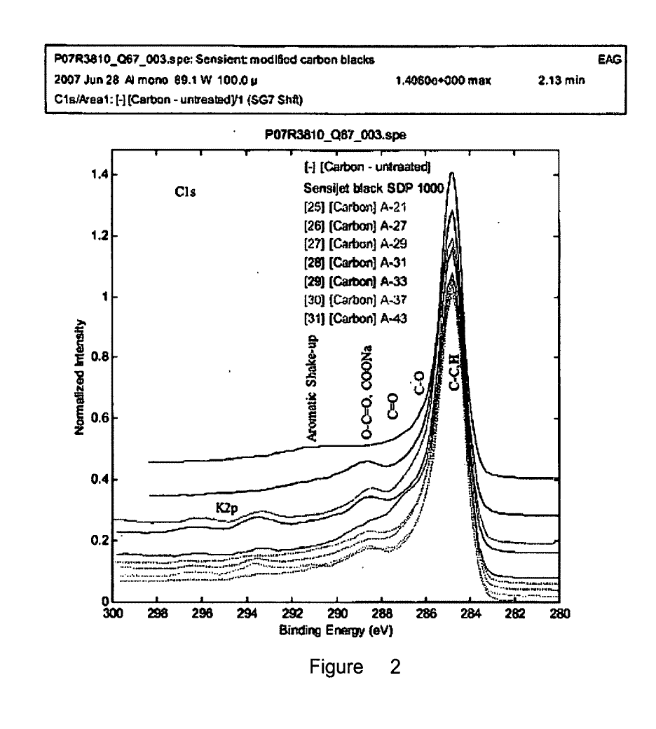

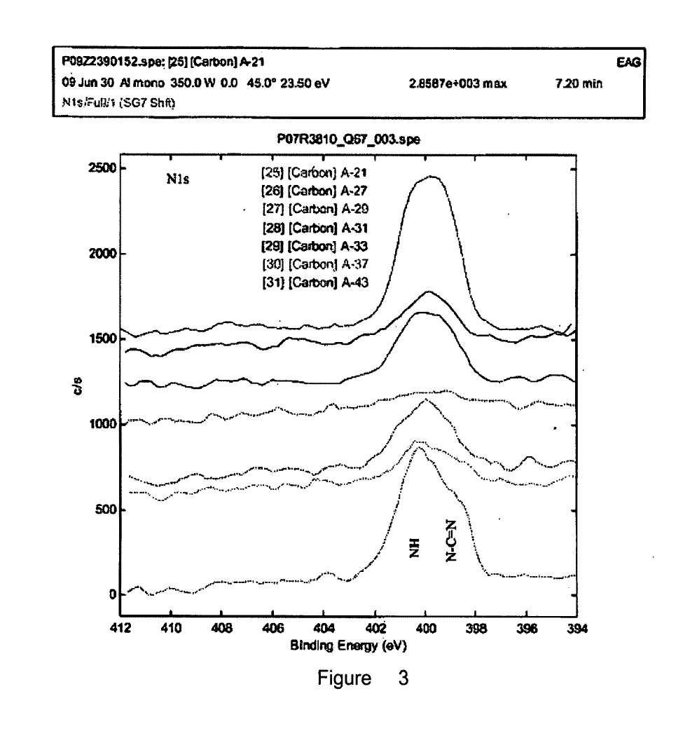

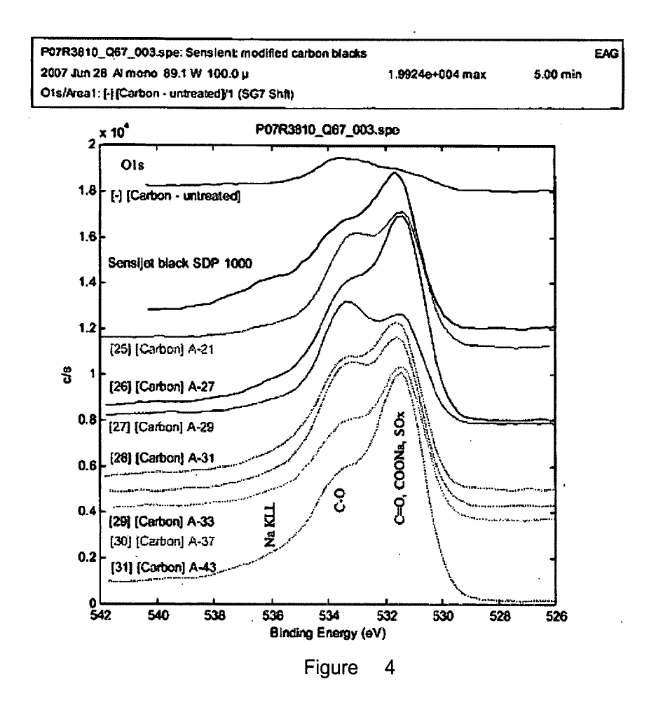

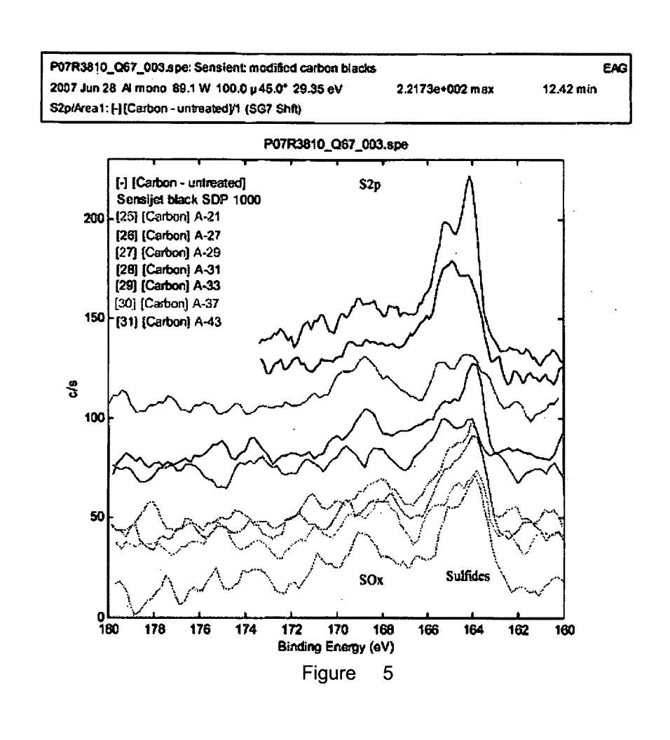

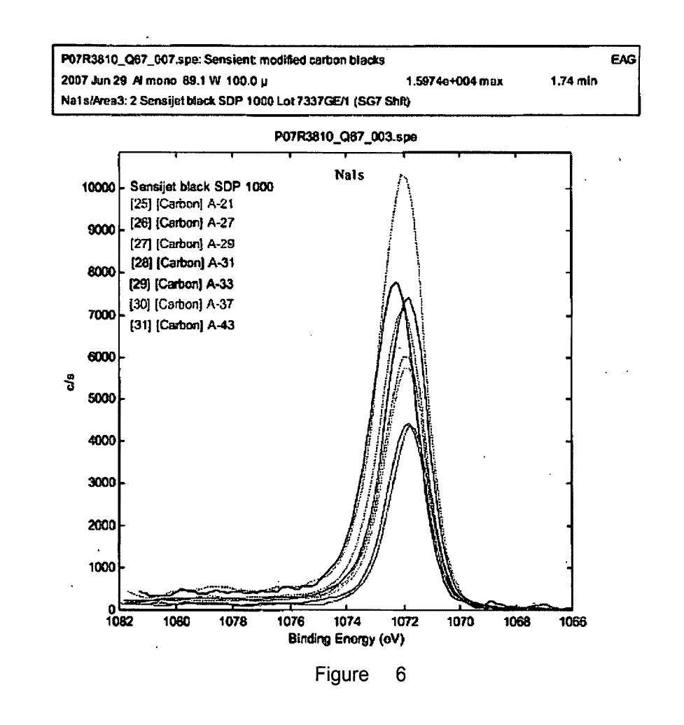

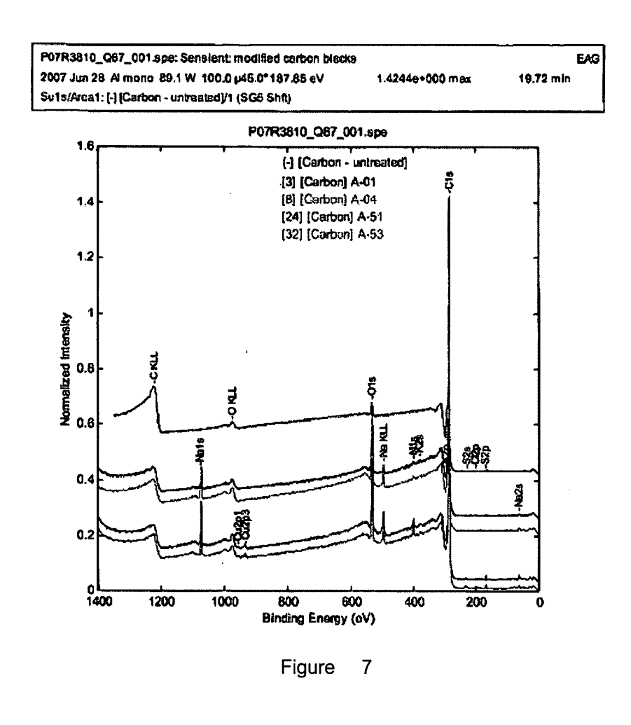

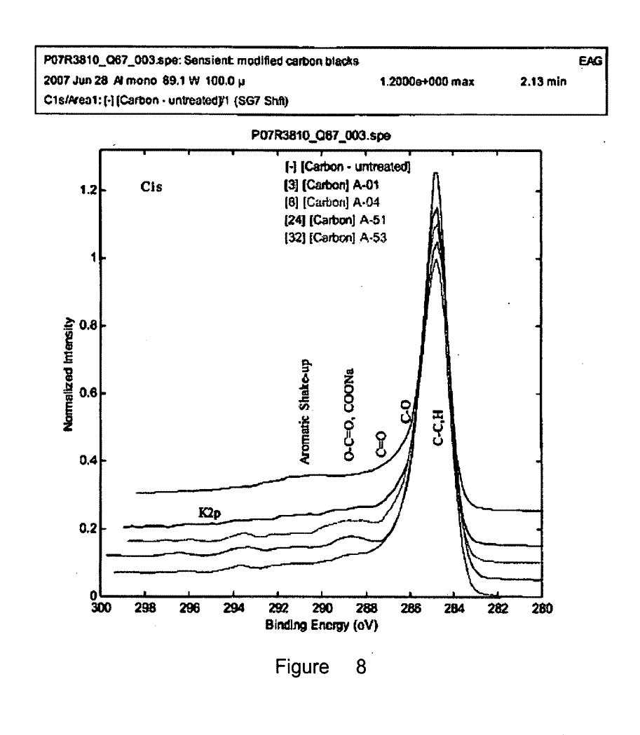

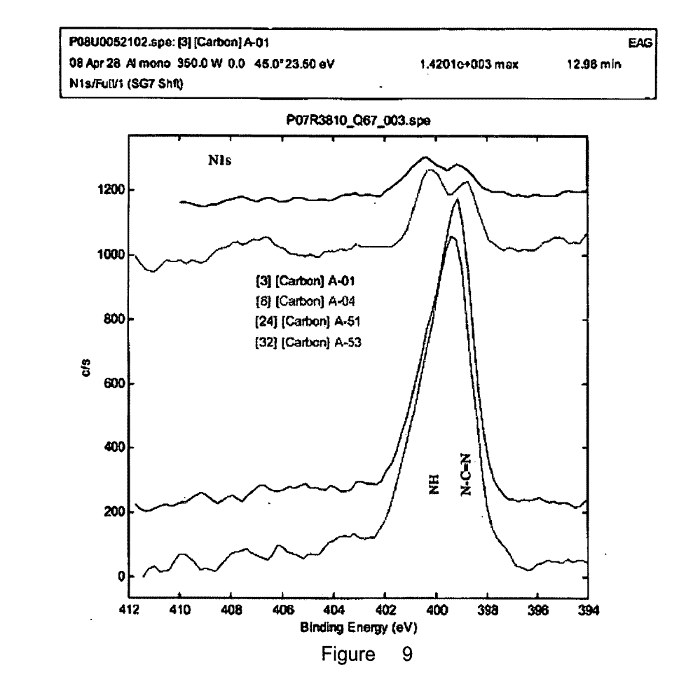

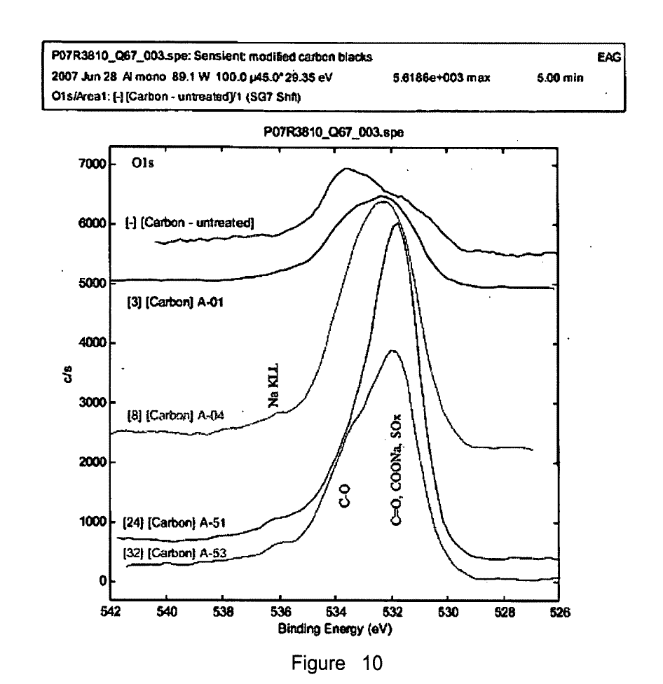

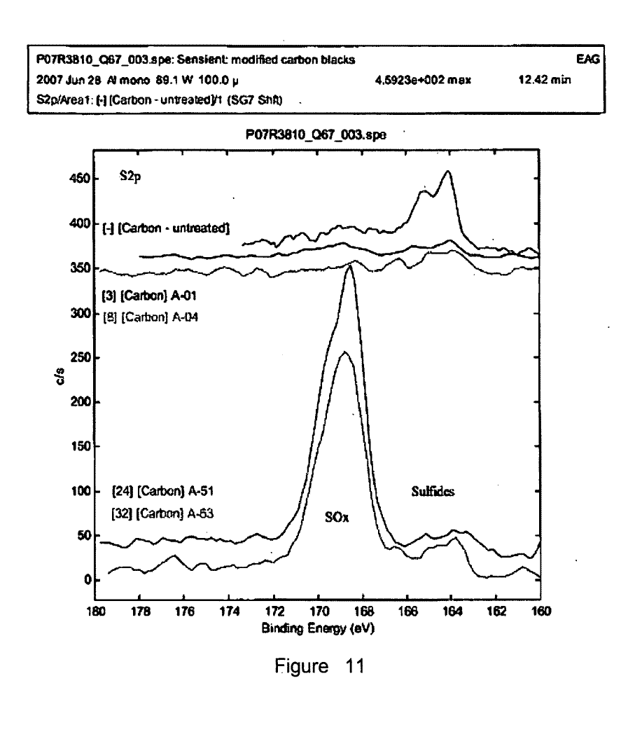

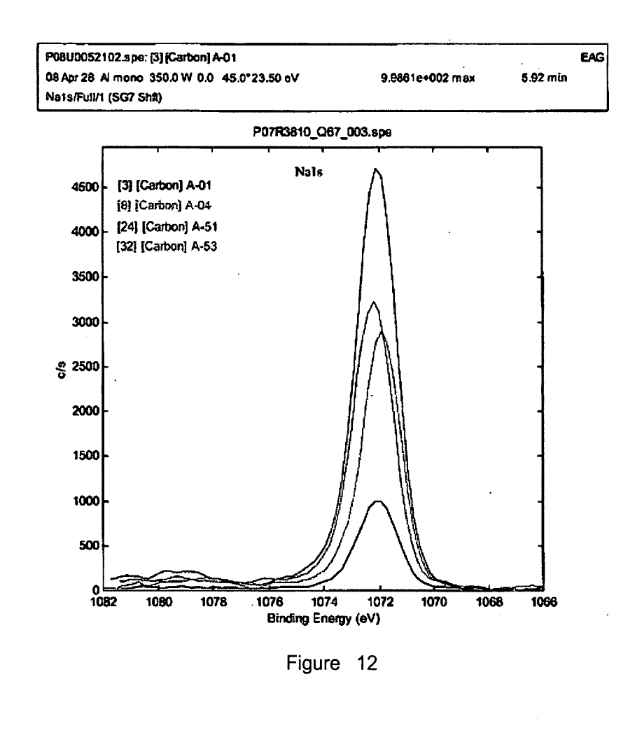

The XPS results disclosed in Example 37 indicate that the surface modification as disclosed yields a modified carbon black with an increase in surface sodium, as COONa, in about 1.4 to 5.3 atomic %. The XPS results for untreated carbon blacks, Sensijet SDP 1000 carbon and carbon blacks from Examples 3, 8, 24, 25-31, and 32 is displayed in FIGS. 1-12.

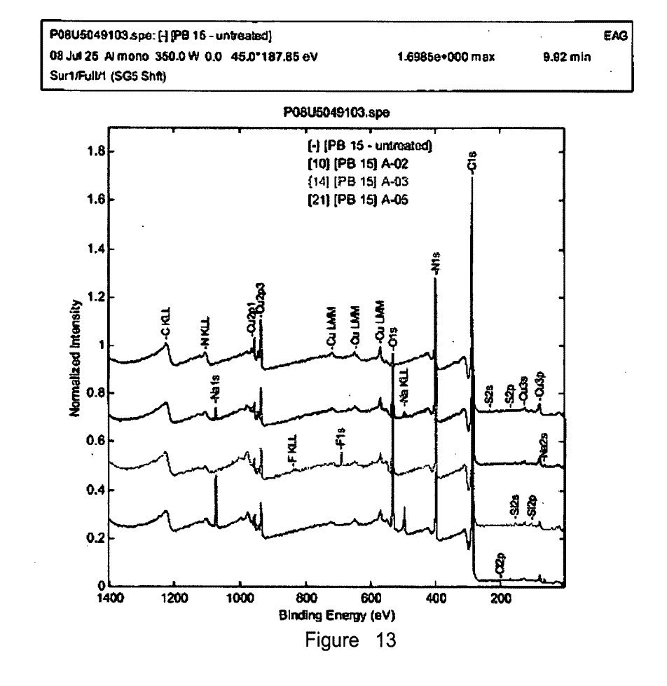

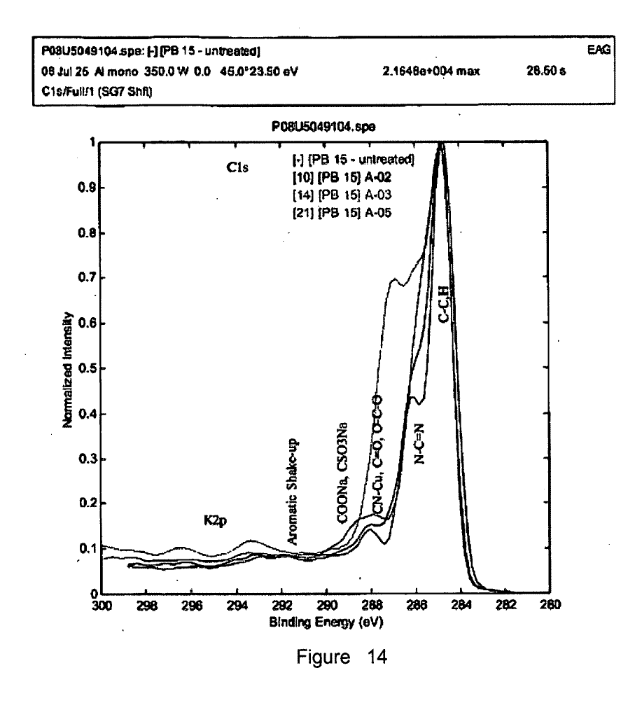

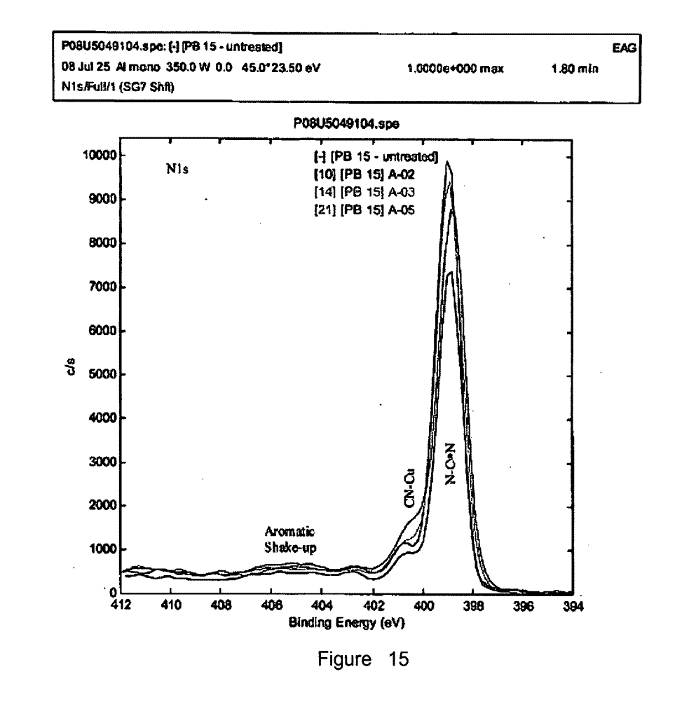

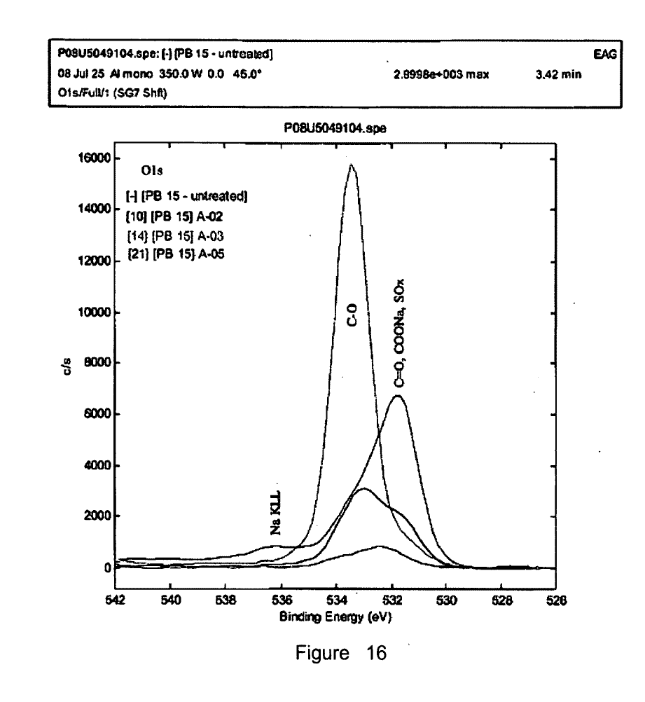

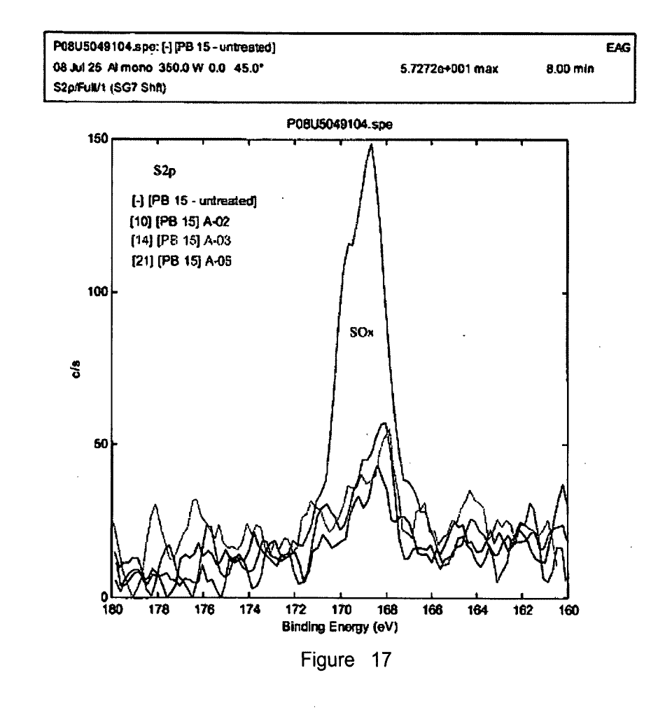

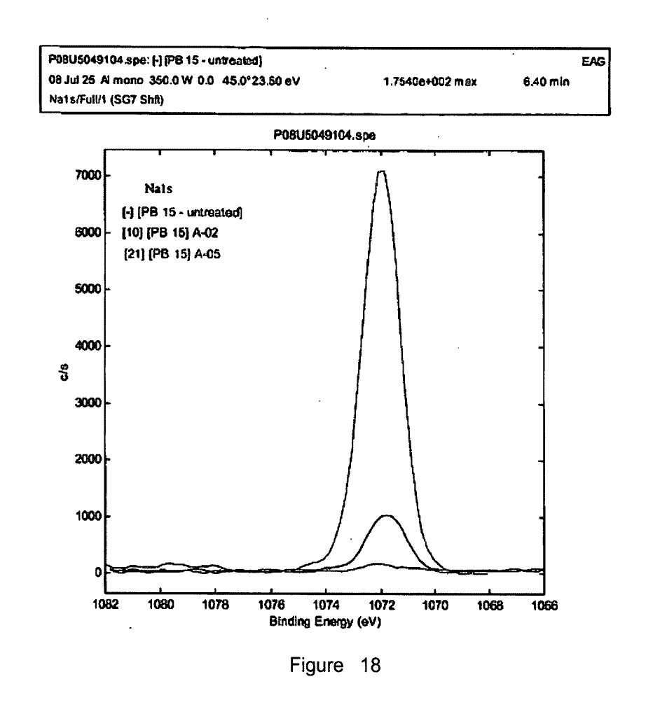

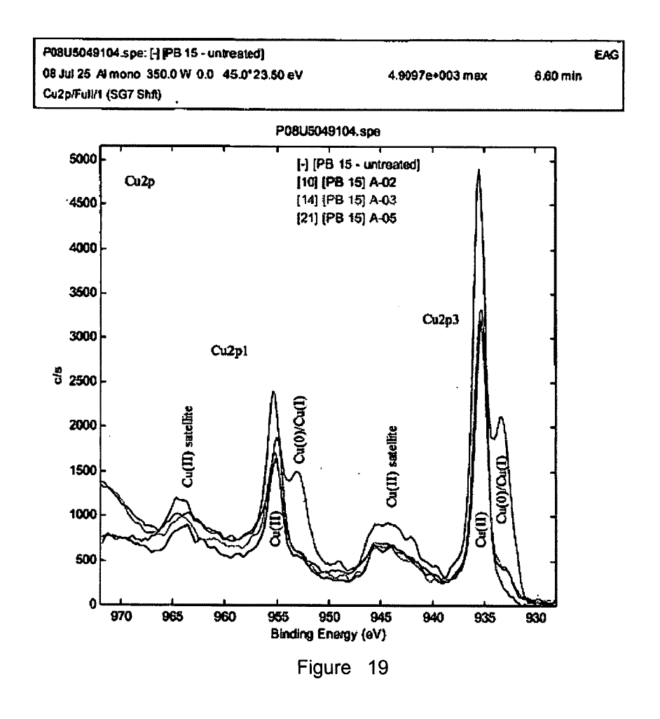

The XPS results disclosed in Example 37 indicate that surface modification as disclosed yields a modified Pigment Blue No. 15 with significantly higher surface sodium content (0.8 to 4.2 atomic %) compared to a low concentration of 0.1 atomic % in the untreated pigment. The XPS results for untreated Pigment Blue No. 15 and Pigment Blue No. 15 from Examples 10, 14, and 21 are displayed in FIGS. 13-19.

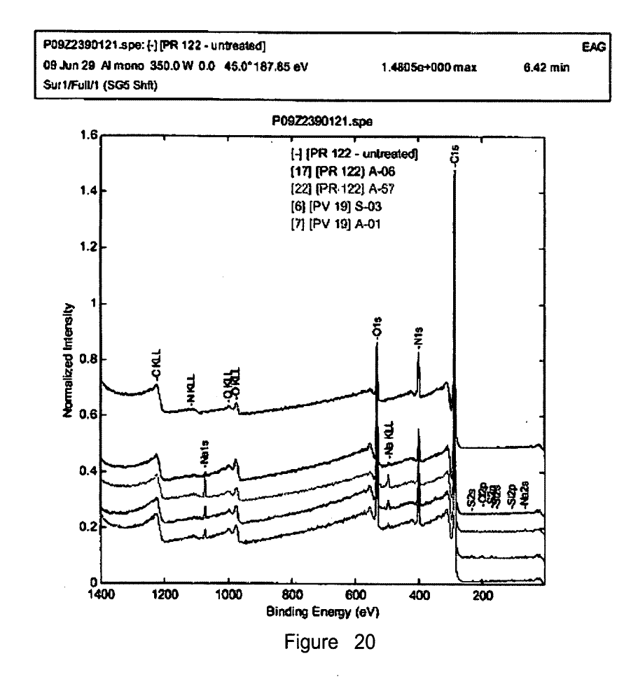

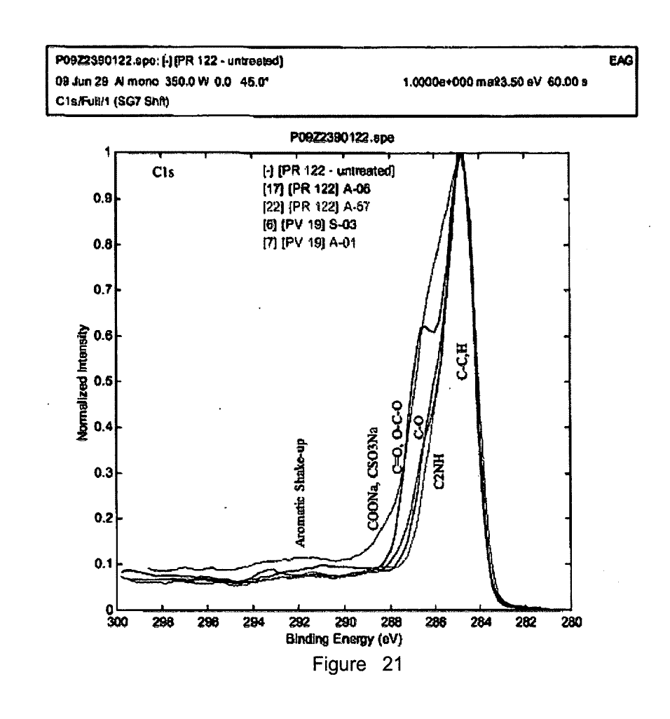

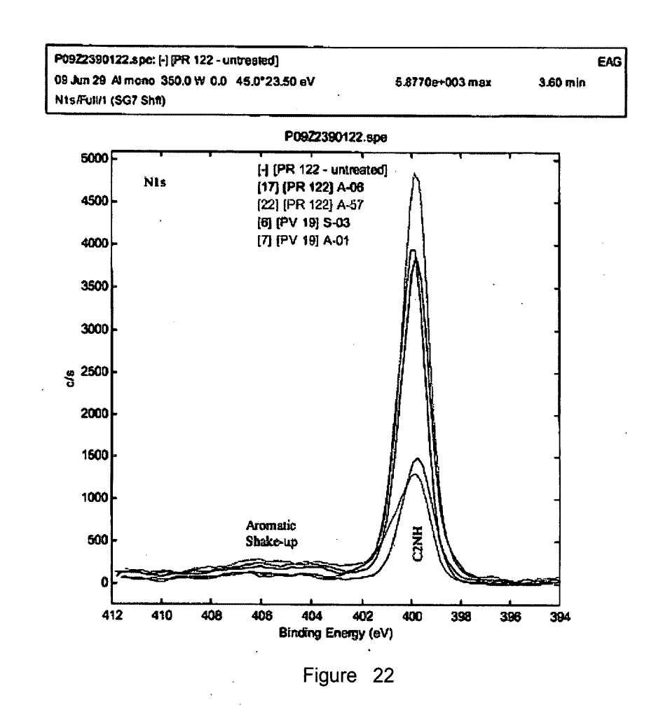

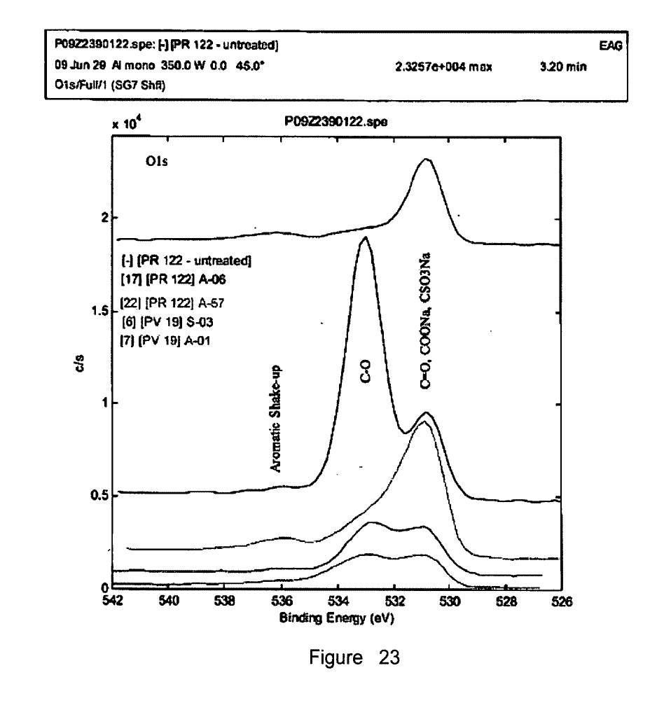

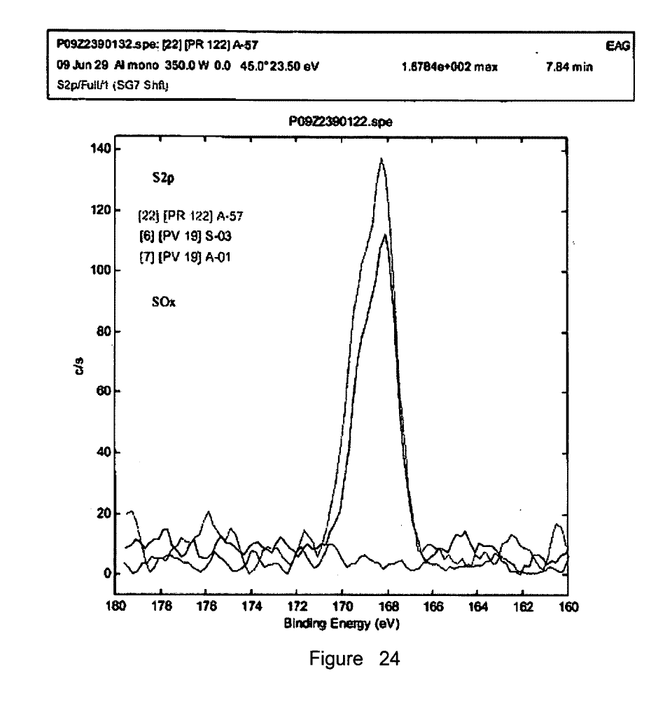

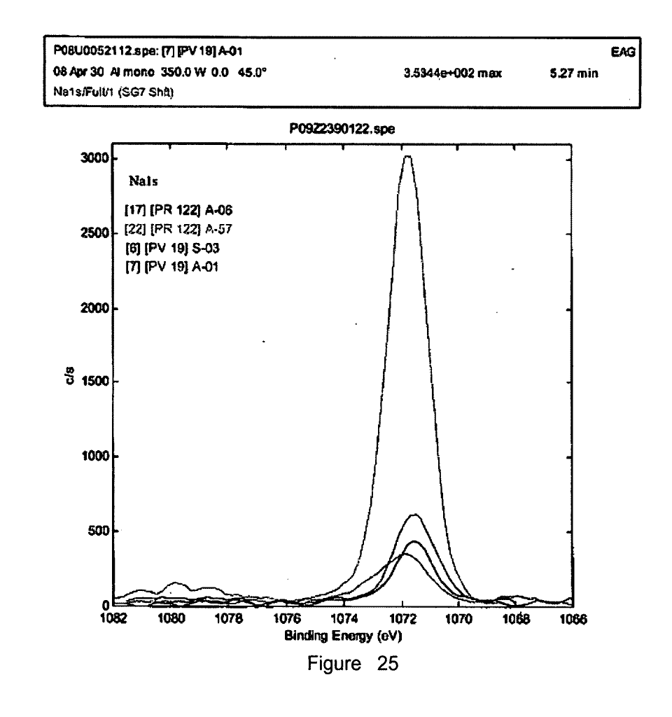

The XPS results disclosed in Example 37 indicate that the surface modification as disclosed yields a modified Pigment Red No. 122 with a surface sodium present at concentrations in the range of 0.3-1.6 atomic % while in comparison the untreated pigment has none. The XPS results for untreated Pigment Red No. 122, Pigment Red No. 122 from Examples 17 and 22, and Pigment Violet 19 from Examples 6 and 7 are displayed in FIGS. 20-25.

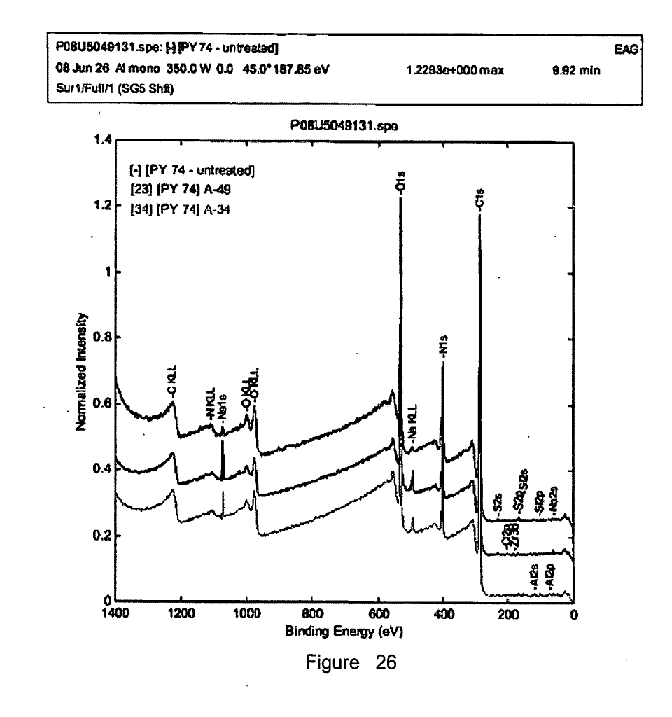

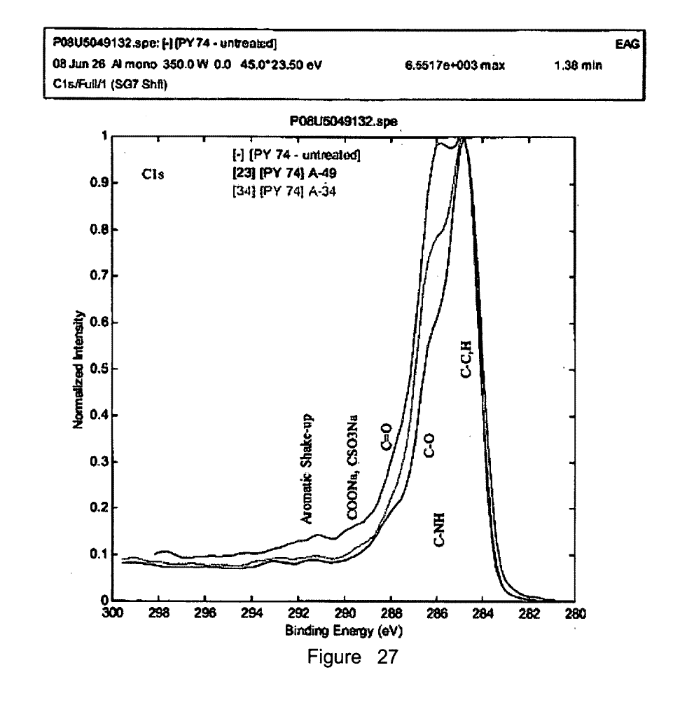

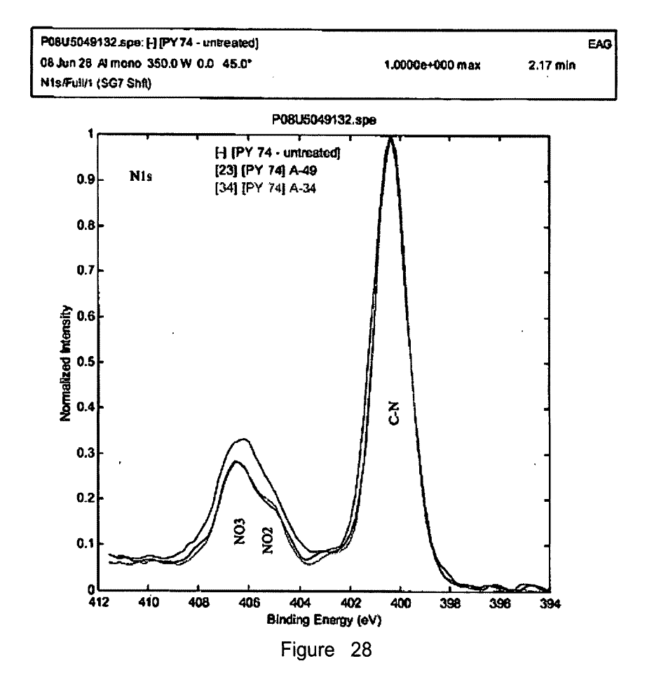

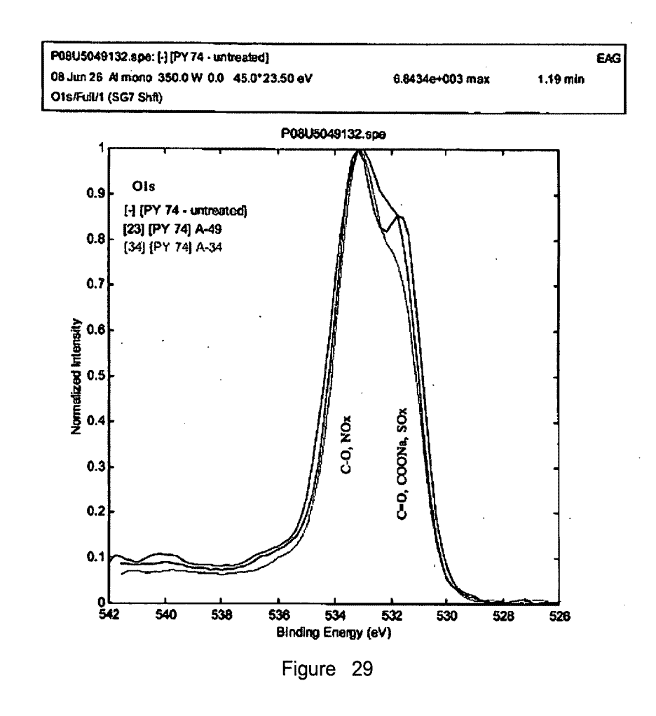

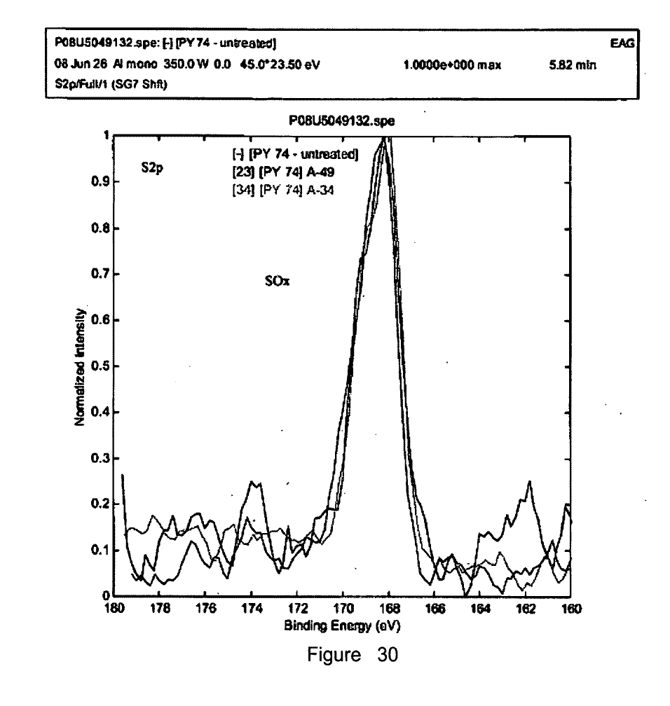

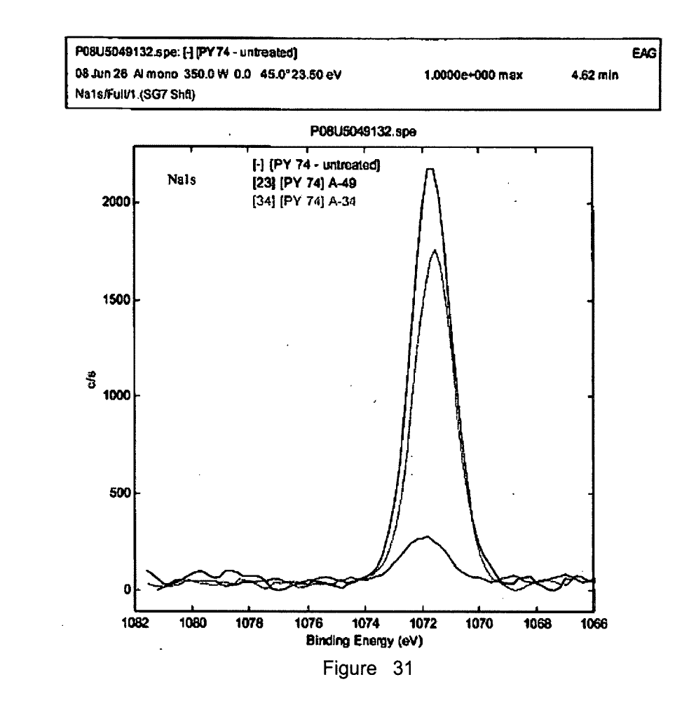

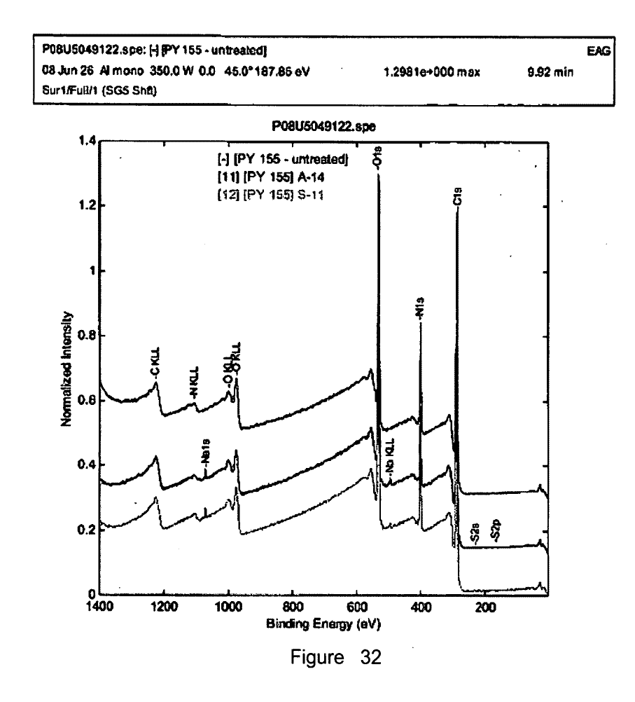

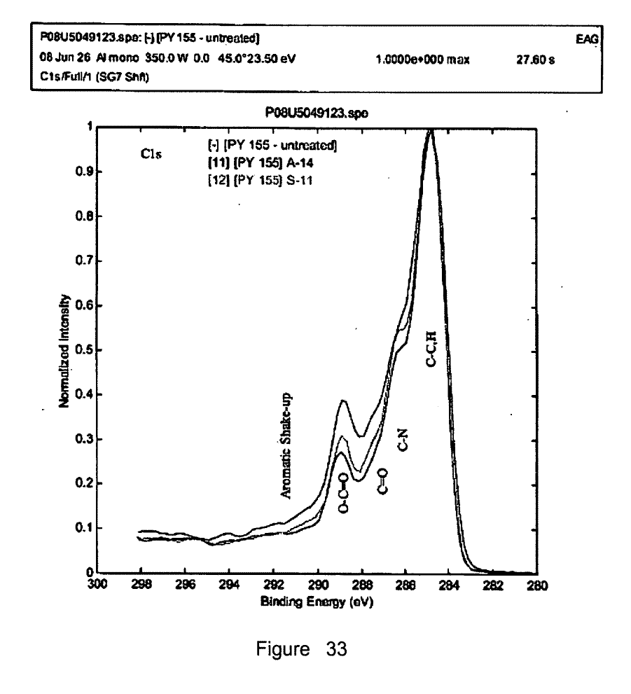

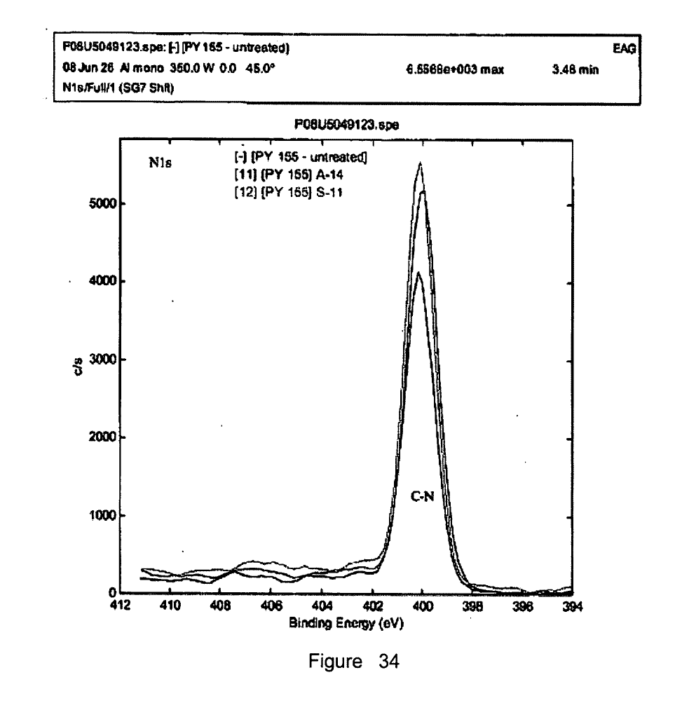

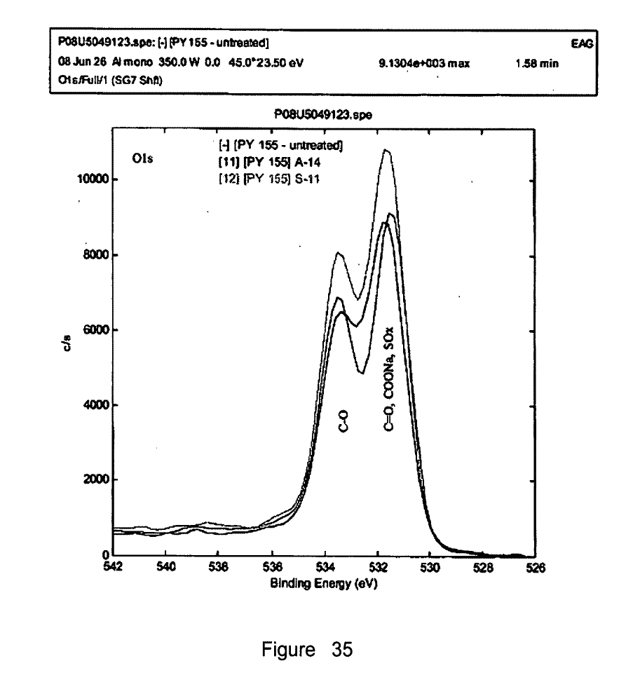

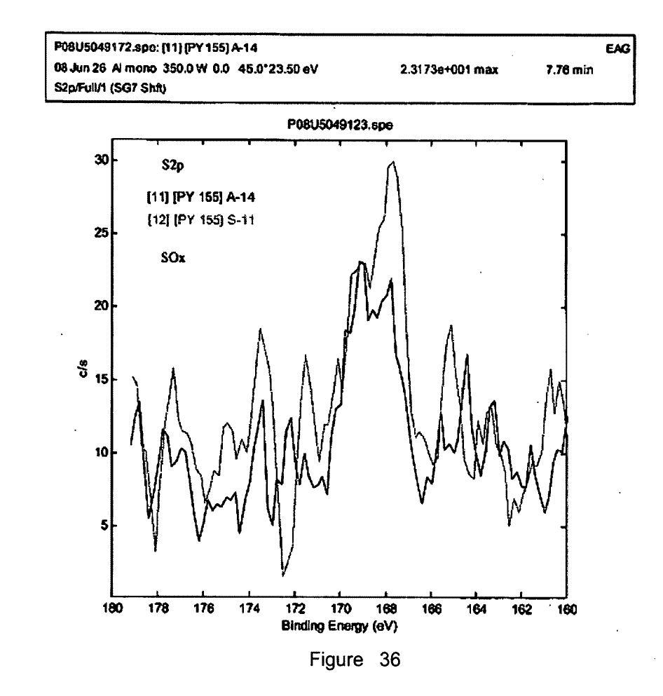

The XPS disclosed in Example 37 indicate that the surface modification as disclosed yields a modified Pigment Yellow No. 74 with a surface sodium in the atomic ratio of 1.0 to 1.6% which is expected to be present as COONa/CSO₃Na. In contrast, in the untreated pigment the surface sodium is only about 0.3. The XPS results for untreated Pigment Yellow No. 74 and Pigment Yellow No. 74 from Examples 23 and 34 are displayed in FIGS. 26-31. The XPS results for untreated Pigment Yellow No. 155 and for Pigment Yellow No. 155 from Examples 11 and 12 are displayed in FIGS. 32-36.

The level of Na is one measure of charge groups present on the pigment. Higher levels of Na may result from the surface modification of pigments. The levels of Na disclosed in the preceding paragraphs for modified pigments may indicate the ability to create a stable pigment dispersion with modified pigments of the present invention. The degree to which a modified pigment dispersion is stable may depend on the amount of charge groups present on the pigment, which can be indicated by the levels of sodium. These results may indicate that pigment dispersions prepared according to the present invention may be stable as a result of the mechanism of attachment.

Applications of Modified Pigments

The self-dispersing pigment according to the present invention may be used in a number of end use applications. These uses include, but are not limited to, coatings, paints, papers, adhesives, latexes, toners, textiles, fibers, plastics, and inks. Specific examples include, without limitation, printing ink for paper, textiles, fibers, metal deco and plastics, wood stains, writing instruments, and color filters. The self-dispersing pigments produced by the process of the invention are particularly well-suited for use in printing applications and wood stains. In one example, an inkjet ink incorporating a pigment of the present invention may be useful in high quality prints in an inkjet photo printer. The self-dispersing pigment according to the present invention may also be used in cosmetic applications, such as, without limitation, mascaras, eye liner, spray-on hair mascara, aqueous nail polish, and hair coloring or hair dyes.

One aspect of the present invention relates to inkjet ink formulations using the self-dispersing pigment described above. Inkjet formulations containing such pigments may do at least one of the following: 1) provide uniform, bleed-free images with high resolution and high density on print media; 2) not cause nozzle clogging which typically occurs due to drying of the ink at a distal end of a nozzle; 3) rapidly dry on paper; 4) exhibit good lightfastness and waterfastness; 5) demonstrate good long-term storage stability; and 6) demonstrate print characteristics which are independent of the paper quality.

The ink compositions of the present invention may be prepared by combining the above modified pigments with an aqueous vehicle and any suitable additives. The amount of modified pigment (by weight) in the ink composition is at least about 0.1%, particularly at least about 10%, and more particularly at least about 20%. Furthermore, the amount of modified pigment (by weight) in the ink composition is less than or equal to about 12%, particularly less than or equal to about 8%, and more particularly less than or equal to about 5%. This includes embodiments where the amount of modified pigment (by weight) in the ink composition is present in an amount ranging from about 2% to about 12%.

The aqueous vehicle may comprise water or water in combination with one or more water-soluble organic solvents. Water-soluble organic solvents may be combined with water to make up the aqueous vehicle. Water-soluble organic solvents may include alcohols, polyhydric alcohols such as ethylene glycol, ketones and ketone alcohols such as acetone and diacetone alcohol, ethers such as tetrahydrofuran and dioxane, lower alkyl ethers of polyhydric alcohols, such as ethylene glycol monomethyl (or monoethyl)ether, nitrogen-containing solvents such as pyrrolidone, N-methyl-2-pyrrolidone, sulfur-containing solvents such as thiodiethanol, sugars and derivatives thereof such as glucose, an oxyethylene adduct of glycerin; and an oxyethylene adduct of diglycerin. The water-soluble organic solvents may be used alone or in combination. If a mixture of water and a water-soluble organic solvent is used, the amount of water-soluble organic solvent (by weight) in the ink composition is at least about 5%, particularly at least about 15%, and more particularly at least about 25%. Furthermore, the amount of water-soluble organic solvent (by weight) in the ink composition is less than or equal to about 50%, particularly less than or equal to about 30%, and more particularly less than or equal to about 15%. This includes embodiments where the amount of water-soluble organic solvent (by weight) in the ink composition is about 5% to about 30%. The amount of water in the ink composition is at least about 40%, particularly at least about 50%, and more particularly at least about 60%. Furthermore, the amount of water (by weight) in the ink composition is less than or equal to about 90%, particularly less than or equal to about 80%, and more particularly less than or equal to about 70%. This includes embodiments where the amount of water (by weight) in the ink composition is about 40% to about 80%.

Additives may be incorporated into the aqueous vehicle to impart any number of desired properties, such as might be needed to adapt the ink to the requirements of a particular inkjet printer or to provide a balance of light stability, smear resistance, viscosity, surface tension, coating penetration, optical density, adhesion, highlighter resistance or crust resistance. Penetrants, for example, may be added to reduce bleed, improve wetting of the print media, and otherwise improve overall performance of the print image. Examples of penetrants may include, but are not limited to, alkyl alcohols having 1 to 4 carbon atoms, such as ethanol, glycol ethers, such as ethylene glycol monomethyl ether, diols such as 1,2-alkyl diols, formamide, acetamide, dimethylsulfoxide, sorbitol and sulfolane. The penetrants may be used alone or in combination. The amount of penetrant (by weight) in the ink composition ranges from 0% to about 60%, particularly from about 2% to about 40%, and more particularly from about 5% to about 20%. This includes embodiments where the amount of penetrant (by weight) in the ink composition is present in an amount ranging from about 10% to about 15%.

Surfactants may be added to the aqueous medium to reduce the surface tension of the ink composition. The surfactants may be anionic surfactants, non-ionic surfactants and/or cationic surfactants. Suitable surfactants may include those listed below and in U.S. Pat. No. 5,116,409 issued May 26, 1992, U.S. Pat. No. 5,861,447 issued Jan. 19, 1999, and U.S. Pat. No. 6,849,111 issued Feb. 1, 2005, each of which is hereby incorporated by reference.

Surfactants are commercially available under various well-known trade names, such as the PLURONIC® series (BASF Corporation, Parsippany, N.J.), the TETRONIC® series (BASF Corporation, Parsippany, N.J.), the ARQUAD® series (Akzo Chemical Inc., Chicago, Ill.), the TRITON® series (Union Carbide Corp., Danbury, Conn.), the SURFONIC® series (Texaco Chemical Company, Houston, Tex.), the ETHOQUAD® series (Akzo Chemical Inc., Chicago, Ill.), the ARMEEN® series (Akzo Chemical Inc., Chicago, Ill.), the ICONOL® series (BASF Corporation, Parsippany, N.J.), the SURFYNOL® series (Air Products and Chemicals, Inc. Allentown, Pa.), and the ETHOMEEN® series (Akzo Chemical Inc., Chicago, Ill.), to name a few.

The surfactants may be used alone or in combination. The amount of surfactant (by weight) in the ink composition may range from 0% to about 10%, particularly from about 0.1% to about 10%, and more particularly from about 0.3% to about 5%. This includes embodiments where the amount of surfactant (by weight) in the ink composition may range from about 0.1% to about 8%.

One or more humectants may be added to the aqueous vehicle to prevent clogging, caused by drying out during periods of latency, of inkjet nozzles. Humectants may be selected from materials having high hygroscopicity and water-solubility. Examples of humectants include, but are not limited to, polyols such as glycerol, lactams such as 2-pyrrolidone, urea compounds such as urea, 1,3-dimethylimidazolidinone, saccharides such as sorbitol, 1,4-cyclohexanedimethanol, 1-methyl-2-piperidone, N-ethylacetamide, 3-amino-1,2-propanediol, ethylene carbonate; butyrolacetone and Liponic EG-1. There are no particular limitations on the amount used of the humectant, but in general the amount of humectant (by weight) in the ink composition may range from 0% to about 30%, particularly from about 1% to about 15%, and more particularly from about 5% to about 10%.

Polymers may be added to the ink composition to improve the water-fastness, rub and highlighter fastness of the images on print media. Suitable polymers may include, but are not limited to, polyvinyl alcohol, polyester, polyestermelamine, styrene-acrylic acid copolymers, styrene-maleic acid copolymers, styrene-maleic acid-alkyl acrylate copolymers, styrene-metacrylic acid copolymers, styrene-metacrylic acid-alkyl acrylate copolymers, styrene-maleic half ester copolymers, vinyl-naphthalene-acrylic acid copolymers, vinyl naphthalene-maleic acid copolymers and salts thereof. The amount of polymer (by weight) in the ink composition may range from 0% to about 5%, particularly from about 0.1% to about 3%, and more particularly from about 0.2% to about 2.5%. This includes embodiments where the amount of polymer (by weight) in the ink composition may range from about 0.1% to about 3.0%.

Ink compositions of the present invention may be buffered to a desired pH using any number of pH modifiers. Suitable pH modifiers may include alkali hydroxides, alkali carbonates and bicarbonates, triethylamine, dimethylethanolamine, triethanolamine, nitric acid, hydrochloric acid, and sulfuric acid. The pH modifiers may be used alone or in combination. The amount of pH modifier (by weight) in the ink composition may range from 0% to about 3.0%, particularly from about 0.1% to about 2.0%, and more particularly from about 0.5% to about 1.5%. This includes embodiments where the amount of pH modifier (by weight) in the ink composition ranges from about 0.2% to about 2.5%.

Preservatives, such as biocides and fungicides, may also be added to the ink composition. Examples of suitable preservatives include sodium benzoate, pentachlorophenol sodium, 2-pyridinethiol-1-oxide sodium, sodium sorbate, sodium dehydroacetate, benzisothiazolinone, 1,2-dibenzothiazolin-3-one, methylisothiazolinone and chloromethylisothiazolinone. Commercially available biocides include UCARCIDE® 250 (available from Union Carbide Company), Proxel® CRL, Proxel® BDN, Proxel® GXL, Proxel® XL-2, Proxel® TN (available from Arch Chemicals, Smyrna, Ga.), Dowicides® (Dow Chemical, Midland, Mich.), Nuosept® (Huls America, Inc., Piscataway, N.J.), Omidines® (Olin Corp., Cheshire, Conn.), Nopcocides® (Henkel Corp., Ambler, Pa.), Troysans® (Troy Chemical Corp., Newark, N.J.), and XBINX® (PMC Specialties Group, Inc., Cincinnati, Ohio). The preservatives may be used alone or in combination. The amount of preservatives (by weight) in the ink composition may range from 0% to about 1.5%, particularly from about 0.05% to about 1.0%, and more particularly from about 0.1% to about 0.3%. This includes embodiments where the amount of preservative (by weight) in the ink composition may range from about 0.05% to about 0.5%.

The ink composition may contain one or more viscosity modifiers. Viscosity modifiers may include rosin compounds, alginic acid compounds, polyvinyl alcohol, hydroxypropyl cellulose, carboxymethyl cellulose, hydroxyethyl cellulose, methyl cellulose, salts of polyacrylic acid, polyvinyl pyrrolidone, gum arabic and starch. The amount of viscosity modifier (by weight) in the ink composition may range from 0% to about 10%, particularly from about 0.5% to about 8%, and more particularly from about 1% to about 5%. This includes embodiments where the amount of viscosity modifier (by weight) in the ink composition may range from about 1% to about 7%.

Other additives which may be incorporated into the aqueous vehicle may also include antioxidants, ultraviolet absorbers, chelating agents, electric conductivity adjusters, viscosity modifiers, oxygen absorbers, anti-kogation agents, anti-curling agents, anti-bleed agents, defoamers, and buffers. The ink compositions of the present invention may contain one or more colorants in addition to the pigment dispersion of the present invention.

The ink compositions of the present invention are particularly suited for use as an ink composition for inkjet printing wherein droplets of the ink composition are ejected from a printing apparatus and deposited onto a substrate to generate an image. Suitable printing apparatus include, but are not limited to, Continuous Ink Jet (CU), prop-on-Demand Valve (DoD Valve), prop-on-Demand Piezo-Electric (DoD Piezo) and Thermal Ink Jet (TIJ). Similarly, any suitable substrate may be employed including plain papers, bonded papers, coated papers, transparency materials, textile materials, plastics, polymeric films and inorganic substrates. However, it should be recognized by those skilled in the art that the above ink compositions may also have use in other applications including, but not limited to, general writing utensil applications and stamp applications.

The ink compositions of the present invention may be used alone, or with a color underlay, to produce a black image or in combination with other ink compositions to produce a color image. In some embodiments, the ink composition of the present invention is used in combination with other ink composition(s), such as a cyan ink, a magenta ink and/or a yellow ink. In other embodiments, a cyan ink, a magenta ink and a yellow ink are overprinted to form a black image and this printing is used in combination with the printing of the black ink of the present invention.

### Wood Stains

Another aspect of the present invention relates to aqueous formulations using the self-dispersing pigment described above as wood stains and coatings. Wood stain formulations containing such pigments may exhibit at least one of the following properties: 1) good wood absorption and adhesion; 2) good transparency; and 3) excellent water and light resistance.

Water resistance is measured by difference in measured DE* values of wood stain in dipped areas versus control. Lower DE* values may indicate higher water resistance. If DE* is small it may mean that there is minimal to no color change due to degradation or loss. For example, lower DE* values may indicate higher water resistance as seen with carboxy modified pigment dispersions. The DE* value of wood stains comprising the surface modified pigment of the present invention may be from about 0 to about 3, suitably about 0 to about 1.5. Delta E is the difference between two colors. L, a, and b values are measurements based on spherical color. +L=white, −L=black, +a=red, −a=green, +b=yellow, −b=blue. C is chroma (saturation) and H=Hue. Readings are measured using a spectrophotometer. Delta E=√(L₁−L₂)²+(a₁−a₂)²+(b₁−b₂)².

### Coatings

Coating formulations containing such pigments may exhibit at least one of the following properties: 1) good adhesion to substrates such as metal, paper, glass, plastic, and wood; 2) ease of application and drying; 3) good weather fastness, water and light resistance; 4) good gloss retention; and 5) good chemical and flocculation resistance.

As with water resistance, resistance to strong acids and bases of coatings are measured as the difference in DE* value of spotted versus control. The DE* value of coatings comprising the surface modified pigment of the present invention may be from about 0 to about 48, suitably about 0 to about 5.

### Color Filters

Another aspect of the present invention relates to aqueous formulations using the self-dispersing pigment described above in color filters. Color filters find application in display imaging areas including, without limitation, desktop monitor/laptop screens, LCD TV screens, cell phone display panels, digital camera screens, and GPS panels. Color filter formulations containing pigments of the present invention may exhibit at least one of the following properties: 1) good adhesion to glass and plastic film substrates; 2) good transparency; 3) ease of application and drying; and 4) good heat and light resistance.

The transmission values of a specific color filter is measured to determine its usefulness. The color filters may have maximum transmittance in a narrow band to provide the most utility.

In one embodiment, color filter formulations comprising carbon black may have no transmission bands, color filter formulations comprising magenta pigment dispersions may have a lowest transmission in the about 520 to about 560 nm range, color filter formulations comprising yellow pigment dispersions may have a lowest transmission in the about 400 to about 480 nm range, and color filter formulations comprising cyan pigment dispersions may have the lowest transmission in the about 600 to about 680 nm range.

### Textile Printing

Another aspect of the present invention relates to aqueous formulations using the self-dispersing pigment described above in textile printing applications. Textile printing formulations containing pigments of the present invention may exhibit at least one of the following properties: 1) good adhesion to textile fabrics such as cotton, nylon, polyester, wool, polyacrylic, or blends of the same; 2) ease of application and drying; 3) good water and light resistance; and 4) good washfastness.

The wash and water fastness properties of dyed textile may be measured by the difference in DE* value of a control versus a treated fabric.

The DE* value of a textile printing composition comprising a surface modified pigment of the present invention may be from about 0 to about 12, suitably from about 0.1 to about 8.0.

### Cosmetic Applications

Another aspect of the present invention relates to formulations using the self-dispersing pigments described above in cosmetic applications. Cosmetic applications may include those directed to, without limitation, the face, eyes, lips, hair, skin, and nails. Cosmetic applications may include, without limitation, mascaras, eye liner, spray-on hair mascara, aqueous nail polish, brush-on-brow, eye shadows, lipsticks, blushers and rouge, make-up, foundation, and hair coloring or hair dyes. The self dispersing pigment dispersions may be easy to incorporate into any aqueous phase portion of a cosmetic formula as they blend easily with polyols and preservatives. Better compatibility with silicones, esters (such as, without limitation, CCT), waxes (such as, without limitation, carnauba wax), and solvents (such as, without limitation, isododecane) helps in the emulsification and yields stable products. The self-dispersed pigments enable a formulator to create a product with higher color strength at equivalent pigment load than with use of a conventional pigment dispersion using a glycerine-water dispersion. The fluidity of the product allows the formulator the flexibility for even higher pigment loads which will enhance the product's pay-off leading to fewer strokes on application.

The properties of mascara comprising self-dispersed pigments of the present invention can be evaluated visually by applying the mascara evenly to the skin and comparing it side-by-side with mascaras that do not comprise self-dispersed pigments of the present invention.

### EXAMPLES

Exemplary embodiments of the present invention are provided in the following examples. The following examples are presented to illustrate the present invention and to assist one of ordinary skill in making and using the same. The examples are not intended in any way to otherwise limit the scope of the invention.

### Example 1

### Pigment Dispersion

### Example of Preparation of a Cyanuryl Tris Adduct with Sulfanilic Acid and Use in the Surface Modification of a Pigment

A solution of sulfanilic acid (114 g) in DI water (310 g), calcium free sodium hydroxide (32 g) and sodium bicarbonate (55 g) at a pH of 8.5 was added to a stirred mixture of cyanuric chloride (40.2 g, available from Lonza Walkersville, Inc., Walkersville, Md.), ice (570 g) and DI water (480 g) in three stages controlling the temperature <0° C., <3° C. and <10° C., respectively. After the addition, the pH was 7.1 and the reaction mixture was heated to 90° C. over 4.5 hours to get 1000 g of a clear liquid.

### Example 2

### Pigment Dispersion

### Example of Preparation of a Cyanuryl Tris Adduct with 4-Aminobenzoic Acid and Use in the Surface Modification of a Pigment

A solution of 4-aminobenzoic acid (90.1 g) in DI water (300 g), calcium free sodium hydroxide (30 g) and sodium bicarbonate (55 g) at a pH of 7.2 was added to a stirred mixture of cyanuric chloride (40.2 g, available from Lonza Walkersville, Inc., Walkersville, Md.), ice (550 g) and DI water (500 g) in three stages controlling the temperature <0° C., <3° C. and <10° C., respectively. After the addition, the pH was 7.1 and the reaction mixture was heated to 92° C. over 3 hours to get 901 g of a clear liquid.

### Example 3

### Pigment Dispersion

### Example of Converting a Polymer Stabilized Dispersion to a Self Dispersed Pigment Dispersion with Cyanuryl Tris Adduct with 4-Aminobenzoic Acid/Sulfanilic Acid

A resin stabilized 15% dispersion of Pigment Black (Carbon black) Sensijet® Black PB 094 available from Sensient Imaging Technologies, Inc., 300 g, was slowly added to a mixture of 157 g of the Tris 4-ABA reagent described in Example 2 and 300 g of DI water.

After one half hour, the reaction mixture was heated to 51° C. A solution of 16.8 g potassium persulfate and 15 g sodium bicarbonate in hot, 50° C. DI water (300 g) was introduced slowly while the pH was maintained between 7.5 and 9.0 with the addition of calcium free sodium hydroxide. After the addition of potassium persulfate solution, the reaction mixture was heated to 80° C. [Step 1]. The dissolved impurities were removed by ultrafiltration until the chloride and sulfate content of the feed sample were less than 50 ppm. The product was then concentrated to 16.7% solids and mixed with (0.3%, wt/wt) Proxel GXL (available from Arch Chemicals, Smyrna, Ga.). Finally, the product (300 g) was centrifuged at 10,000 rpm for 5 minutes and then filtered through a 0.7 micron GF filter.

### Examples 4-7

Examples 4-7 were prepared following the same process as set forth above for Example 3, except in some instances, the Tris reagent from Example 1 was used (shown in the table).

TABLE 1
Examples of attaching molecules to a polymer dispersed
pigment via a Cyanuric adduct.

| Example | Pigment | | Tris Adduct | | NaHCO₃ | K₂S₂O₈ | Equivalent | | Step 1 | |
|---|---|---|---|---|---|---|---|---|---|---|
| [#] | Type | (g) | Type | (g) | (g) | (g) | SA/4-ABA | (g) | ° C. | h |
| 4 | PB15² | 300 | 4-ABA | 177.8 | 16.7 | 16.8 | 4-ABA | 10.26 | 90 | 0.5 |
| 5 | PB15² | 300 | SA | 215 | 20 | 24 | SA | 14.1 | 95 | 0.5 |
| 6 | PV19³ | 500 | SA | 100 | 11 | 32 | SA | 11.4 | 85 | 3 |
| 7 | PV19³ | 300 | 4-ABA | 90 | 10 | 11 | 4-ABA | 8.85 | 83 | 0.5 |

¹Sensijet ® Black PB 094 from Sensient Imaging Technologies, Inc
²Sensijet ® Cyan PB15:3 from Sensient Imaging Technologies, Inc.
³Sensijet ® Magenta PV19 from Sensient Imaging Technologies, Inc

### Example 8

### Pigment Dispersion

### Example of Milling to (<100 nm) and Converting a Polymer Stabilized Dispersion to a Self Stabilized Dispersion Using a Cyanuryl Tris Adduct with 4-Aminobenzoic Acid/Sulfanilic Acid

A resin stabilized 15% dispersion of Pigment Black (Carbon black) Sensijet® Black PB 094, available from Sensient Imaging Technologies, Inc., 5 Kg, was milled with Buhler Micro Media P1 Perl Mill with 0.1 mm YTZ ceramic media for 4 hours at 43° C. As required additional styrene acrylic copolymer dispersant Joncryl 678 (available from BASF) was added (358 g) to prevent gross agglomeration.

A part (400 g) was slowly added to a mixture of 80.8 g of the Tris 4-ABA reagent described in Example 2 and 725 g of DI water.

After one half hour, the reaction mixture was heated to 47° C. A solution of 20 g potassium persulfate and 18 g sodium bicarbonate in hot, 50° C. DI water (300 g) was introduced slowly while the pH was maintained between 7.5 and 9.0 with the addition of calcium free sodium hydroxide (17 g). After the addition of potassium persulfate solution, the reaction mixture was heated to 95° C. [Step 1]. The dissolved impurities were removed by ultrafiltration until the chloride and sulfate content of the feed sample were less than 50 ppm. The product was then concentrated to 16.7% solids and mixed with (0.3%, wt/wt) Proxel GXL (available from Arch Chemicals, Smyrna, Ga.). Finally, the product (352 g) was centrifuged at 10,000 rpm for 5 minutes and then filtered through a 0.7 micron GF filter.

### Examples 9-13

Examples 9-13 were prepared following the same process as set forth above for Example 8, except in example 12, the Tris reagent from Example 1 was used (shown in the table).

TABLE 2
Examples of attaching molecules to a polymer dispersed pigment after particle
size reduction with a micro media mill, using a Cyanuric adduct.

| Example | Pigment | | Tris Adduct | | NaHCO₃ | K₂S₂O₈ | Equivalent | | Step 1 | |
|---|---|---|---|---|---|---|---|---|---|---|
| [#] | Type | (g) | Type | (g) | (g) | (g) | SA/4-ABA | (g) | ° C. | h |
| 9 | Carbon¹ | 500 | 4-ABA | 223.2 | 45 | 49 | 4-ABA | 22.3 | 85 | 0.5 |
| 10 | PB15² | 300 | 4-ABA | 177.8 | 16.7 | 16.8 | 4-ABA | 10.26 | 90 | 0.5 |
| 11 | PY155⁴ | 312 | 4-ABA | 90 | 13.6 | 11 | 4-ABA | 6.0 | 80 | 0.5 |
| 12 | PY155⁴ | 200 | SA | 332 | 20.1 | 11 | SA | 20 | 90 | 0.5 |
| 13 | PV19³ | 600 | 4-ABA | 73 | 5.5 | 16 | 4-ABA | 7.3 | 85 | 1 |

⁴Sensijet ® Yellow PY155 from Sensient Imaging Technologies, Inc

### Examples 14-20

Examples 14-21 were prepared following the same process as set forth for Example 3, using Lemantex pigment dispersions available from Sensient Imaging Technologies—Specialty Inks and colors, Switzerland.

TABLE 3
Additional examples of attaching molecules to a polymer dispersed
pigment via a Cyanuric adduct.

| | | | | | | 4-ABA | | |
|---|---|---|---|---|---|---|---|---|
| Example | Pigment | | Tris Adduct | NaHCO₃ | K₂S₂O₈ | Equivalent | Step 1 | |
| [#] | Type | (g) | (g) | (g) | (g) | (g) | ° C. | h |
| 14 | PB15⁵ | 463 | 60 | 4.2 | 13.5 | 6 | 55 | 16 |
| 15 | PB60⁶ | 496 | 66.3 | 5.0 | 16.2 | 6.6 | 55 | 16 |
| 16 | PG7⁷ | 519 | 66.3 | 5.4 | 16.2 | 6.6 | 54 | 16 |
| 17 | PR122⁸ | 400 | 101.8 | 7.1 | 22 | 10.1 | 55 | 16 |
| 18 | PR254⁹ | 440 | 71.8 | 5.6 | 17.6 | 7.2 | 54 | 16 |
| 19 | PY83¹⁰ | 403 | 65.3 | 5.0 | 16.2 | 6.5 | 53 | 16 |
| 20 | PY120¹¹ | 601 | 66.5 | 5.1 | 16.3 | 6.7 | 56 | 16 |

⁵Cyan PB 15:3 [11.2%] from Sensient Specialty Inks and Colors, Switzerland
⁶Blue PB 60 [13.5%] from Sensient Specialty Inks and Colors, Switzerland
⁷Green PG7 [10.7%] from Sensient Specialty Inks and Colors, Switzerland
⁸Magenta PR122 [12.9%] from Sensient Specialty Inks and Colors, Switzerland
⁹Red PR254 [11.6%] from Sensient Specialty Inks and Colors, Switzerland
¹⁰Orange PY83 [13.3%] from Sensient Specialty Inks and Colors, Switzerland
¹¹Yellow PY120 [10%] from Sensient Specialty Inks and Colors, Switzerland

### Example 21

Tris 4-ABA reagent described in Example 2 (200 g at 10.2% conc) is used to wet commercial Pigment Blue No. 15 wet cake¹² (100 g @100% solids) available from Clariant Colors (Charlotte, N.C.) along with 100 g of Sensijet Direct Blue 199 (available from Sensient Colors Inc, St. Louis, Mo.). The mixture is heated to 50° C. and stirred with 10 g of 25% solution of calcium free sodium hydroxide, an aqueous solution containing 1 g of Joncryl HPD 296 resin (available from BASF), and 20 g of poly(styrene-co-maleic anhydride) cumene terminated resin MW ˜1700 (available from Aldrich chemicals) to get an uniform mix. It is then milled with Hockmeyer Basket Mill with 0.2 mm YTZ ceramic media for 12 hours. A mixture of potassium persulfate (20 g) and sodium bicarbonate (13 g) is added to facilitate attachment. As needed, a defoamer to control the foaming and calcium free sodium hydroxide solution to hold the pH 9-10 are added.

The milled product above is then removed from the mill, combined with mill rinses and heated overnight at 50-55° C. to complete the reaction. The dissolved impurities are removed by ultrafiltration until the chloride and sulfate content of the feed sample are less than 50 ppm. The product is then concentrated to 18% solids and mixed with (0.3%, wt/wt) Proxel GXL (available from Arch Chemicals, Smyrna, Ga.). Finally, the product is centrifuged at 5,000 rpm for 20 minutes and then filtered through a 0.7 micron GF filter.

### Examples 22-24

Examples 22-24 were prepared following the same process as set forth above for Example 21 using different colored pigments as shown in the Table 4. The Sensijet Direct Blue 199 was replaced with Sensijet Acid Red 289 Na solution in example #22 and with 20 g of Surfynol CT-131 in example #23.

TABLE 4

| | | | | | | Final |
|---|---|---|---|---|---|---|
| | | | 4-ABA | | | conc of |
| Example | Pigment | | equivalent | NaHCO₃ | K₂S₂O₈ | pigment |
| [#] | Type | (g) | (g) | (g) | (g) | % |
| 22 | PR122¹³ | 120 | 60 | 0 | 39.6 | 15.34 |
| 23 | PY74¹⁴ | 120 | 20 | 0 | 21 | 15.53 |
| 24 | Carbon¹⁵ | 100 | 60 | 0 | 40 | 12.7 |

¹²Pigment Blue No. 15:3 wet cake, 45% solids, from Clariant Colors (Charlotte, NC)
¹³Pigment Red 122 from CIBA (Newport, DE)
¹⁴Pigment Yellow 74 from SUN (Parsippany, NJ)
¹⁵Carbon Black powder from Degussa, (Akron, OH)

### Example 25

Example of attaching Surfonamines to oxidized self-dispersed carbon.

Sensijet Black SDP1000 carbon¹⁶ dispersion (40 g @ 100%) is diluted to 5% concentration and mixed with the tris reagent¹⁷ in a 1 L beaker. The mixture is stirred with a regular overhead stirrer at 300-500 rpm while it is heated to 50° C. on a hot plate. To the heated pigment mixture, is added dropwise, a solution of potassium persulfate (6.81 g) and sodium bicarbonate (2.12 g) while adjusting the pH to 8-9 with Ca free sodium hydroxide. The reaction mixture is stirred at 50° C. for 20 hours and purified by ultrafiltration until the chloride and sulfate content of the feed sample are less than 50 ppm. The product is then concentrated to 11.1% solids and mixed with (0.3%, wt/wt) Proxel GXL (available from Arch Chemicals, Smyrna, Ga.). ¹⁶ Oxidized Carbon Black self dispersed liquid @ 14-14.5%, available from Sensient Colors Inc (St. Louis, Mo.)

### Examples 26-31

Example of attaching Surfonamines and PEHA to oxidized self-dispersed carbon. Examples 26-31 are prepared using Sensijet SDP1000¹⁶ oxidized carbon black pigment and tris reagents containing various Surfonamines and pentaethylhexamine as given in Table 5.

TABLE 5

| | Pigment | | | | Final conc |
|---|---|---|---|---|---|
| Example | SDP 1000 | Tris Adduct | NaHCO₃ | K₂S₂O₈ | of pigment |
| [#] | (g) | (g) | (g) | (g) | % |
| 26 | 40 | 7.96¹⁸ | 2.12 | 6.81 | 8.21 |
| 27 | 40 | 4.95¹⁹ | 0.54 | 1.62 | 15.1 |
| 28 | 40 | 4.48²⁰ | 0.61 | 1.95 | 15.83 |
| 29 | 40 | 4.09²¹ | 1.01 | 3.24 | 12.03 |
| 30 | 40 | 6.0²² | 2.74 | 8.81 | 12.86 |
| 31 | 40 | 4.89²³ | 2.12 | 6.81 | 10.35 |

¹⁷Tris adduct is obtained by reacting cyanuric chloride with 1 equiv. Surfonamine B-30 and 2 equiv. 4-aminobenzoic acid.
¹⁸Tris adduct is obtained by reacting cyanuric chloride with 1 equiv. Surfonamine B-60 and 2 equiv. 4-aminobenzoic acid.
¹⁹Tris adduct is obtained by reacting cyanuric chloride with 1 equiv. Surfonamine B-30 and 2 equiv Surfonamine L-100.
²⁰Tris adduct is obtained by reacting cyanuric chloride with 3 equiv. Surfonamine B-60.
²¹Tris adduct is obtained by reacting cyanuric chloride with 3 equiv. Surfonamine B-30.
²²Tris adduct is obtained by reacting cyanuric chloride with 3 equiv. 4-aminobenzoic acid. The reaction mixture also contained Joncryl HPD 196 (11.94 g as a 36% solution from BASF).
²³Tris adduct is obtained by reacting cyanuric chloride with 1 equiv. PEHA (pentaethylhexamine) and 2 equiv 4-aminobenzoic acid.

### Example 32

### Pigment Dispersion

### Example of Milling a Pigment with a Polymer Stabilizer and a Polymer of Styrene Co-Acrylic Acid Type Converting to a Self Stabilized Dispersion by the Use of Cyanuryl Tris Adduct with 4-Aminobenzoic Acid

Tris 4-ABA reagent described in Example 2 (400 g at 15% conc) is used to wet 100 g of commercial gas carbon black available from Degussa (Akron, Ohio), with a primary particle size of 20 nm and B.E.T surface area of 160 m²/g, along with 100 g of Sensijet Direct Blue 199 (available from Sensient Colors Inc, St. Louis, Mo.). The mixture is heated to 50° C. and stirred with 10 g of 25% solution of calcium free sodium hydroxide, an aqueous solution containing 1 g of Joncryl HPD 296 resin (available from BASF), and 55.6 g of Joncryl HPD 196 (available from BASF as a 36% solution) to get an uniform mix. It is then milled with Hockmeyer Basket Mill with 0.4 mm YTZ ceramic media for 14 hours. Potassium persulfate (40 g) is added to facilitate attachment. As needed, a defoamer to control the foaming and calcium free sodium hydroxide solution to hold the pH 9-10 are added.

The milled product above is then removed from the mill, combined with mill rinses purified by ultrafiltration until the chloride and sulfate content of the feed sample are less than 50 ppm. The product is then concentrated to 15.6% solids and mixed with (0.3%, wt/wt) Proxel GXL (available from Arch Chemicals, Smyrna, Ga.). Finally, the product is centrifuged at 10,000 rpm for 20 minutes and then filtered through a 0.7 micron GF filter.

### Example 33

### Example of Attaching Polyethyleneimine [PEI] Via Tris Reagents to Conventionally Dispersed Pigment(s)

A resin stabilized 20% dispersion of Sensijet® Magenta PV19³ (300 g) was slowly added to a mixture of 90 g of the Tris 4-ABA reagent described in Example 2 and 250 g of DI water.

A clear solution of Epomine SP-012 (79.5 g, 1200 MW, available from Nippon Shokubai) in 300 g DI water was cooled to 8° C. and mixed with cyanuric chloride (5 g, available from Lonza Walkersville, Inc., Walkersville, Md.) to form 690.6 g of tris epomine reagent, analogues to tris 4-ABA preparation described in Example #2. A part (48 g) of the tris epomine reagent was diluted with DI water (140 g), heated to 38° C. and then added to the treated pigment dispersion above. The reaction mixture was then heated to 51° C. and a solution of 13 g potassium persulfate and 11 g sodium bicarbonate in hot, 50° C. DI water (300 g) was introduced slowly while the pH was maintained between 10 and 10.5 with the addition of calcium free sodium hydroxide. After the addition of potassium persulfate solution, the reaction mixture was heated to 80° C. for one hour. The reaction mixture was held at 55-58° C. for 20 hours. The dissolved impurities were removed by ultrafiltration until the chloride and sulfate content of the feed sample were less than 50 ppm. The product was then concentrated to 15.5% solids and mixed with (0.3%, wt/wt) Proxel GXL (available from Arch Chemicals, Smyrna, Ga.). Finally, the product (300 g) was centrifuged at 10,000 rpm for 20 minutes and then filtered through a 0.7 micron GF filter.

### Example 34

### Pigment Dispersion

### Example of Milling a Pigment with a Grind Aid, a Polymer Stabilizer, and Converting to a Self Stabilized Dispersion by the Use of Cyanuryl Tris Adduct with 4-Aminobenzoic Acid

Surfynol CT-131 (available from Air Products, Allentown, Pa.) (20 g) diluted with 300 g DI water is used to wet and grind commercial Pigment Yellow 74¹⁴ (100 g). It is then milled with Hockmeyer Basket Mill with 0.4 mm YTZ ceramic media for 5 hours. The milled pigment is then treated with tris 4-ABA reagent described in Example 2 (81.4 g at 15.0% conc) according to procedure described in example 3-7. The final product (579.4 g) was centrifuged at 10,000 rpm for 10 minutes and then filtered through a 0.7 micron GF filter.

### Example 35

The example 34 was repeated with Tamol SN (available from Rohm and Haas, Philadelphia, Pa.) as grind aid replacing Surfynol CT-131 and special grade²⁴ of PY 74. ²⁴ Hansa Brilliant Yellow from Clariant Colors (Charlotte, N.C.)

### Example 36

The physical properties of the modified pigments from the examples above are set forth in the following table.

TABLE 6

| Example | Pigment | Solids | | Cl | SO₄ | Viscosity |
|---|---|---|---|---|---|---|
| [#] | Type | (%) | pH | ppm | ppm | Cps |
| 3 | Carbon¹ | 16.7 | 8.5 | 4 | 7 | 3.38 |
| 4 | PB15² | 14.3 | 7.9 | 1 | 5 | 2.7 |
| 5 | PB15² | 14.6 | 7.7 | 5 | 4 | 2.8 |
| 6 | PV19³ | 16.1 | 8.2 | 1 | 19 | 2.86 |
| 7 | PV19³ | 15.6 | 8.3 | 16 | 11 | 2.6 |
| 8 | Carbon¹ | 15.9 | 9.4 | 7 | 2 | 7.42 |
| 9 | Carbon¹ | 18.2 | 9.2 | 38 | 11 | 10.8 |
| 10 | PB15² | 17.6 | 8.3 | 20 | 9 | 9.15 |
| 11 | PY155⁴ | 14.8 | 8.1 | 2 | 4 | 2.89 |
| 12 | PY155⁴ | 14.7 | 8.2 | 1 | 3 | 2.90 |
| 13 | PV19³ | 14.9 | 8.5 | 9 | 25 | 5.69 |
| 14 | PB15⁵ | 24.6 | 8.5 | 5 | 4 | 5.77 |
| 15 | PB60⁶ | 16.4 | 8.3 | 10 | 98 | 4.52 |
| 16 | PG7⁷ | 13.2 | 9.7 | 5 | 12 | 2.04 |
| 17 | PR122⁸ | 17.7 | 8.1 | 300 | 182 | 4.27 |
| 18 | PR254⁹ | 17.0 | 6.1 | 1 | 7 | 3.75 |
| 19 | PY83¹⁰ | 19.5 | 8.3 | 15 | 3 | 5.2 |
| 20 | PY120¹¹ | 16.6 | 7.8 | 40 | 8 | 3.2 |
| 21 | PB15¹² | 18.3 | 9.7 | 6 | 1 | 4.9 |
| 22 | PR122¹³ | 15.3 | 8.9 | 1 | 3 | 2.2 |
| 23 | PY74¹⁴ | 15.5 | 9.9 | 9 | 51 | 2.19 |
| 24 | Carbon¹⁵ | 16.4 | 9.3 | 26 | 28 | 5.52 |
| 25 | Carbon¹⁶ | 11.1 | 9.7 | 32 | 136 | 2.4 |
| 26 | Carbon¹⁶ | 8.2 | 9.6 | 7 | 38 | 1.8 |
| 27 | Carbon¹⁶ | 15.1 | 9.2 | 5 | <1 | 2.56 |
| 28 | Carbon¹⁶ | 15.8 | 9.3 | 4 | 3 | 4.08 |
| 29 | Carbon¹⁶ | 12.0 | 9.4 | <1 | 12 | 1.88 |
| 30 | Carbon¹⁶ | 12.9 | 9.5 | 2 | 1 | 3.85 |
| 31 | Carbon¹⁶ | 10.4 | 11.1 | 16 | 10 | 1.60 |
| 32 | Carbon¹⁵ | 12.5 | 9.0 | 8 | 25 | 3.44 |
| 33 | PV19³ | 15.1 | 8.9 | 4 | 2 | 2.88 |
| 34 | PY74¹⁴ | 14.5 | 8.7 | 7 | 37 | 1.66 |
| 35 | PY74²⁴ | 14.4 | 8.3 | 2 | 4 | 1.97 |

Analytical Results of Pigment Dispersions.

| | | | | | | | Heavy |
|---|---|---|---|---|---|---|---|
| Example | Pigment | Conductivity | Surface tension | Na | K | S | metals²⁵ |
| [#] | Type | μS | Dynes/cm | ppm | Ppm | ppm | ppm |
| 3 | Carbon¹ | 2460 | 56.8 | 1131 | 699 | 487 | 38.8 |
| 4 | PB15² | 2500 | 43 | 869 | 660 | 293 | 35.4 |
| 5 | PB15² | 2500 | 41 | 725 | 630 | 722 | 31.4 |
| 6 | PV19³ | 2120 | 34.2 | 887 | 679 | 456 | 28.7 |
| 7 | PV19³ | 2000 | 39 | 746 | 449 | 132 | 33.3 |
| 8 | Carbon¹ | 3820 | 61.4 | 2863 | 1438 | 473 | 144 |
| 9 | Carbon¹ | 3750 | 62.5 | 3103 | 1586 | 605 | 178.9 |
| 10 | PB15² | 2870 | 41.4 | 1328 | 923 | 279 | 70.6 |
| 11 | PY155⁴ | 3820 | 36.8 | 1099 | 698 | 159 | 8.7 |
| 12 | PY155⁴ | 3720 | 39.7 | 360 | 256 | 356 | 45.9 |
| 13 | PV19³ | 1793 | 42.5 | 880 | 460 | 169 | 58.2 |
| 14 | *PB15⁵ | 4710 | 46.8 | 459 | 3000* | 477 | 185.6 |
| 15 | PB60⁶ | 2840 | 34.3 | 829 | 195 | 2 | 20.3 |
| 16 | PG7⁷ | 1422 | 39.8 | 587 | 33 | 186 | 63 |
| 17 | PR122⁸ | 1327 | 46.6 | 543 | 433 | 192 | 95.2 |
| 18 | PR254⁹ | 949 | 49.1 | 277 | 280 | 217 | 8.8 |
| 19 | PY83¹⁰ | 1403 | 44.6 | 462 | 358 | 189 | 147 |
| 20 | PY120¹¹ | 954 | 42 | 477 | 438 | 215 | 62 |
| 21 | PB15¹² | 4140 | 51.3 | 2796 | 469 | 653 | 55.3 |
| 22 | PR122¹³ | 4970 | 54.3 | 2859 | 577 | 685 | 24.2 |
| 23 | PY74¹⁴ | 4270 | 39.3 | 2382 | 526 | 327 | 33.9 |
| 24 | Carbon¹⁵ | 5370 | 40.1 | 2600 | 1060 | — | 16.7 |
| 25 | Carbon¹⁶ | 1181 | 38.0 | 1933 | 1206 | — | 51.7 |
| 26 | Carbon¹⁶ | 1082 | 38.2 | 1798 | 1172 | — | 23.0 |
| 27 | Carbon¹⁶ | 1660 | 34.2 | 2986 | 908 | — | 25.4 |
| 28 | Carbon¹⁶ | 1464 | 40.8 | 4047 | 1204 | — | 27.4 |
| 29 | Carbon¹⁶ | 886 | 37.0 | 2676 | 1427 | — | 23.3 |
| 30 | Carbon¹⁶ | 2970 | 42.7 | 3230 | 1673 | — | 21.8 |
| 31 | Carbon¹⁶ | 1730 | 66.8 | 3429 | 598 | — | 24.3 |
| 32 | Carbon¹⁵ | 4730 | 45.6 | 2261 | 913 | — | 18.0 |
| 33 | PV19³ | 1889 | 35.1 | 825 | 297 | 109 | 21.7 |
| 34 | PY74¹⁴ | 1359 | 40.8 | 591 | 107 | 375 | 21.4 |
| 35 | PY74²⁴ | 2450 | 59.4 | 1252 | 283 | 1596 | 22.2 |

²⁵Sum of Ca, Mg and Fe present as a contaminant in the raw materials and/or formed during the milling process.
*A potassium chelate called Belcene ® was used to remove heavy metals.

### Example 37

### X-Ray Photoelectron Spectroscopy (XPS) Analyses

XPS data were collected and analyzed for Black samples, Cyan samples, Magenta samples, and Yellow samples (Table 7). Dried samples of purified “Tris” reagents were also analyzed for identifying the nature of the groups attached to the pigment surface. The numbers in brackets in the table refer to example numbers.

TABLE 7
XPS of pigment samples.

| Sample | Source |
|---|---|
| [—] Carbon Black | Gas carbon black, available from Degussa, |
| | Akron, OH. |
| [—] Sensijet | Inkjet Grade Carbon Dispersion from Sensient |
| black SDP 1000 | Colors Inc, St. Louis, MO. |
| [3] [Carbon] A-01 | Dispersion from Example #3 with 4-ABA |
| | attachment |
| [8] [Carbon] A-04 | Dispersion from Example #8 with 4-ABA |
| | attachment |
| [24] [Carbon] A-51 | Dispersion from Example #24, 4-ABA and |
| | SMA attachment |
| [25] [Carbon] A-21 | Dispersion from Example #25 oxidized carbon |
| | with 4ABA and Surfonamine B 30 attachment |
| [26] [Carbon] A-27 | Dispersion from Example #26 oxidized carbon |
| | with 4ABA and Surfonamine B 60 attachment |
| [27] [Carbon] A-29 | Dispersion from Example #27 oxidized carbon |
| | with Surfonamine B 30 and L-100 attachment |
| [28] [Carbon] A-31 | Dispersion from Example #28 oxidized carbon |
| | with Surfonamine B 60 attachment |
| [29] [Carbon] A-33 | Dispersion from Example #29 oxidized carbon |
| | with Surfonamine B 30 attachment |
| [30] [Carbon] A-37 | Dispersion from Example #30 oxidized carbon |
| | with Styrene-Acrylate attachment |
| [31] [Carbon] A-43 | Dispersion from Example #31 oxidized carbon |
| | with PEHA attachment |
| [32] [Carbon] A-53 | Dispersion from Example #32 raw carbon with |
| | Styrene-Acrylate attachment |
| [—] PB 15 - untreated | Inkjet Grade Pigment Blue 15:3 from BASF |
| [10] [PB 15] A-02 | Dispersion from Example #10 with 4-ABA |
| | attachment |
| [14] [PB 15] A-03 | Dispersion from Example #14 with 4-ABA |
| | attachment |
| [21] [PB 15] A-05 | Dispersion from Example #21 with 4-ABA and |
| | SMA attachment |
| [—] [PR 122 - untreated] | Inkjet Grade Pigment Red 122 from CIBA |
| [17] [PR 122] A-06 | Dispersion from Example #17 with 4-ABA |
| | attachment |
| [22] [PR 122] A-57 | Dispersion from Example #22 with 4-ABA and |
| | SMA attachment |
| [6] [PV 19] S-03 | Dispersion from Example #6 with SA |
| | attachment |
| [7][PV 19] A-01 | Dispersion from Example #7 with 4-ABA |
| | attachment |
| [—] [PY 74 - untreated] | Inkjet Grade Pigment Yellow 74 from SUN |
| [23] [PY 74] A-49 | Dispersion from Example #23 with 4-ABA and |
| | SMA attachment |
| [34] [PY 74] A-34 | Dispersion from Example #34 with 4-ABA and |
| | alkyne attachment |
| [—] [PY 155 - untreated] | Inkjet Grade Pigment Yellow 155 from Clariant |
| [11 [PY 155] A-14 | Dispersion from Example #11 with 4-ABA |
| | attachment |
| [12] [PY 155] S-11 | Dispersion from Example #12 with SA |
| | attachment |

The XPS data were acquired by EAG Labs (in Chanhassen, Minn.) using a probe beam of focused, monochromatic Al K_{α} radiation. The x-rays generate photoelectrons that are energy analyzed and counted to reveal the atomic composition and chemistry of the sample surface. The escape depth of the photoelectrons limits the depth of analysis to the outer ˜50 Å. The data presented includes low resolution survey scans, which give the full spectrum between 0 and 1400 eV binding energy. Also included in the data are high resolution spectra from selected elements, which provide chemical state information. The spectra are used to obtain surface composition by integrating the areas under the photoelectron peaks and applying empirical sensitivity factors. The XPS data is presented in FIGS. 1-36.

TABLE 8
Analytical Conditions.

| | |
|---|---|
| Instrument: | Physical Electronics 5802 Multitechnique, |
| | Quantum 2000 Scanning XPS |
| X-ray Source: | Monochromatic Al K_{α} 1486.6 eV |
| Analysis Area: | 1.5 mm × 0.6 mm - 5802, 1.2 mm × 0.2 mm - |
| | Quantum 2000 |
| Take-off Angle: | 45° |
| Charge Correction: | C—C, C—H in C1s spectra set to 284.8 eV |
| Charge Neutralization: | Low energy electron and ion floods |

### Tables for Carbon Black Samples

The following tables were normalized to 100% of the elements detected. XPS does not detect H or He. Detection limits are typically between 0.05% and 1.0% for other elements. A dash “-” indicates the element was not detected. High 0 (more than 10 atomic %) and Na (more than 3 atomic %) for modified samples is indicative of a surface COONa bond introduced by oxidation. The S content as sulfide is typical of carbon black which appears to be partially oxidized under modification conditions. The levels of N, Na and K present in all samples, except the unreacted carbon, is a measure of charge groups present either as amino benzoic or maleic acid or acrylic acid or surface carboxylic or sulfonic acid groups as corresponding sodium or potassium salts.

TABLE 9-1
XPS Surface Concentrations of Carbon Black Samples (Atomic %).

| | | | | | | | |
|---|---|---|---|---|---|---|---|
| Sample | C | N | O | Na | S | Cl | K |

Group A (Attachment to Carboxy modified)

| | | | | | | | |
|---|---|---|---|---|---|---|---|
| [—] [Carbon - untreated] | 97.5 | — | 2.4 | — | 0.11 | 0.03 | — |
| Sensijet Black SDP 1000 | 81.4 | — | 13.0 | 5.3 | 0.11 | 0.19 | — |
| [25] [Carbon] A-21 | 79.6 | 2.6 | 13.2 | 3.5 | 0.10 | 0.30 | 0.7 |
| [26] [Carbon] A-27 | 78.8 | 0.5 | 15.1 | 4.7 | 0.04 | 0.14 | 0.8 |
| [27] [Carbon] A-29 | 79.0 | 1.4 | 14.9 | 3.9 | 0.05 | 0.26 | 0.5 |
| [28] [Carbon] A-31 | 80.0 | 0.3 | 13.8 | 4.9 | 0.05 | 0.11 | 0.9 |
| [29] [Carbon] A-33 | 80.7 | 1.0 | 13.5 | 3.9 | 0.04 | 0.11 | 0.7 |
| [30] [Carbon] A-37 | 83.0 | 0.8 | 11.8 | 3.8 | 0.1 | 0.1 | 0.5 |
| [31] [Carbon] A-43 | 82.9 | 1.3 | 10.9 | 4.4 | 0.1 | 0.2 | 0.2 |

Group B (Attachment to Carbon Black)

| | | | | | | | |
|---|---|---|---|---|---|---|---|
| [—] [Carbon - untreated] | 97.5 | — | 2.4 | — | 0.11 | 0.03 | — |
| [3] [Carbon] A-01 | 91.4 | 0.6 | 6.5 | 1.4 | 0.13 | — | — |
| [8] [Carbon] A-04 | 86.7 | 0.9 | 10.1 | 2.0 | 0.07 | 0.06 | — |
| [24] [Carbon] A-51 | 83.3 | 2.4 | 9.3 | 3.5 | 0.8 | 0.1 | 0.4 |
| [32] [Carbon] A-53 | 86.9 | 2.6 | 7.0 | 2.3 | 0.6 | 0.1 | 0.2 |

TABLE 9-2
Carbon Chemistries of Carbon Black Samples (% of total C).

| | | | | | |
|---|---|---|---|---|---|
| | | | | COONa, | Aromatic |
| Sample | C—C,H | C—O* | C═O | O—C═O | Shake-up |

Group A (Attachment to Carboxy modified)

| | | | | | |
|---|---|---|---|---|---|
| [—] [Carbon - | 86 | 3 | 0.7 | 0.2 | 10 |
| untreated] | | | | | |
| Sensijet Black SDP | 77 | 7 | 2 | 5 | 9 |
| 1000 | | | | | |
| [25] [Carbon] A-21 | 79 | 8 | 2 | 6 | 5 |
| [26] [Carbon] A-27 | 78 | 6 | 3 | 6 | 8 |
| [27] [Carbon] A-29 | 75 | 9 | 5 | 5 | 6 |
| [28] [Carbon] A-31 | 81 | 6 | 2 | 5 | 6 |
| [29] [Carbon] A-33 | 80 | 7 | 2 | 5 | 6 |
| [30] [Carbon] A-37 | 84 | 7 | 3 | 5 | 1 |
| [31] [Carbon] A-43 | 80 | 8 | 4 | 5 | 3 |

Group B (Attachment to Carbon Black)

| | | | | | |
|---|---|---|---|---|---|
| [—] [Carbon - | 86 | 3 | 0.7 | 0.2 | 10 |
| untreated] | | | | | |
| [3] [Carbon] A-01 | 91 | 4 | 0.3 | 1.6 | 3 |
| [8] [Carbon] A-04 | 90 | 5 | — | 2.6 | 2 |
| [24] [Carbon] A-51 | 86 | 8 | 0.3 | 4 | 2 |
| [32] [Carbon] A-53 | 88 | 8 | 1 | 2 | 1 |

TABLE 9-3
Nitrogen Chemistries of Carbon Black Samples (% of total N).
Group A (Attachment to Carboxy modified)

| Sample | N—C═N | NH |
|---|---|---|
| [—] [Carbon - untreated] | — | — |
| Sensijet Black SDP 1000 | — | — |
| [25] [Carbon] A-21 | 49 | 51 |
| [26] [Carbon] A-27 | 48 | 52 |
| [27] [Carbon] A-29 | 45 | 55 |
| [28] [Carbon] A-31 | 42 | 58 |
| [29] [Carbon] A-33 | 43 | 57 |
| [30] [Carbon] A-37 | 37 | 63 |
| [31] [Carbon] A-43 | 40 | 60 |

Group B (Attachment to Carbon Black)

| Sample | N—C═N | NH | NO₃ |
|---|---|---|---|
| [—] [Carbon - untreated] | — | — | — |
| [7] [Carbon] A-1 | 40 | 60 | — |
| [6] [Carbon] A-004 | 31 | 43 | 26 |
| [24] [Carbon] A-51 | 71 | 29 | — |
| [32] [Carbon] A-53 | 74 | 26 | — |

TABLE 9-4
Oxygen Chemistries of Carbon Black Samples (% of total O).

| | | |
|---|---|---|
| Sample | C═O, COONa, Sox | C—O |

Group A (Attachment to Carboxy modified)

| | | |
|---|---|---|
| [—] [Carbon - untreated] | 32 | 68 |
| Sensijet Black SDP 1000 | 65 | 35 |
| [25] [Carbon] A-21 | 49 | 51 |
| [26] [Carbon] A-27 | 58 | 42 |
| [27] [Carbon] A-29 | 43 | 57 |
| [28] [Carbon] A-31 | 55 | 45 |
| [29] [Carbon] A-33 | 52 | 48 |
| [30] [Carbon] A-37 | 60 | 40 |
| [31] [Carbon] A-43 | 63 | 37 |

Group B (Attachment to Carbon Black)

| | | |
|---|---|---|
| [—] [Carbon - untreated] | 32 | 68 |
| [3] [Carbon] A-01 | 44 | 56 |
| [8] [Carbon] A-04 | 50 | 50 |
| [24] [Carbon] A-51 | 72 | 28 |
| [32] [Carbon] A-53 | 61 | 39 |

TABLE 9-5
Sulfur Chemistries of Carbon Black Samples (% of total S).

| | | |
|---|---|---|
| Sample | Sulfides | SOx |

Group A (Attachment to Carboxy modified)

| | | |
|---|---|---|
| [—] [Carbon - untreated] | 69 | 31 |
| Sensijet Black SDP 1000 | 82 | 18 |
| [25] [Carbon] A-21 | 49 | 51 |
| [26] [Carbon] A-27 | 100 | 0 |
| [27] [Carbon] A-29 | 100 | 0 |
| [28] [Carbon] A-31 | 100 | 0 |
| [29] [Carbon] A-33 | 100 | 0 |
| [30] [Carbon] A-37 | 59 | 41 |
| [31] [Carbon] A-43 | 61 | 39 |

Group B (Attachment to Carbon Black)

| | | |
|---|---|---|
| [—] [Carbon - untreated] | 69 | 31 |
| [3] [Carbon] A-01 | 53 | 47 |
| [8] [Carbon] A-04 | 100 | — |
| [24] [Carbon] A-51 | 6 | 94 |
| [32] [Carbon] A-53 | 11 | 89 |

The S present in untreated carbon as sulfides was largely oxidized to sulfate/sulfone in all treated samples, adding to the surface charge groups.

### Tables for PB 15 Samples

TABLE 10-1
XPS Surface Concentrations of PB 15 Samples (Atomic %).

| Sample | C | N | O | Na | S | Cl | Cu |
|---|---|---|---|---|---|---|---|
| [—] [PB 15 - untreated] | 78.7 | 17.3 | 1.6 | 0.1 | 0.09 | — | 2.3 |
| [10] [PB 15] A-02 | 77.9 | 13.1 | 6.6 | 0.8 | 0.05 | — | 1.6 |
| [14] [PB 15] A-03 | 72.3 | 11.8 | 11.7 | — | 0.12 | — | 1.5 |
| [21] [PB 15] A-05 | 70.0 | 13.1 | 10.0 | 4.2 | 0.25 | 0.12 | 2.0 |

TABLE 10-2
Carbon Chemistries of PB 15 Samples (% of total C).

| | | | | COONa/ | Aromatic |
|---|---|---|---|---|---|
| Sample | C—C,H | N—C═N* | CN—Cu | CSO3Na | Shake-up |
| [—] [PB | 67 | 22 | 4.7 | 1.1 | 5 |
| 15 - | | | | | |
| untreated] | | | | | |
| [10] [PB 15] | 68 | 23 | 3.7 | 1.5 | 4 |
| A-02 | | | | | |
| [14] [PB 15] | 45 | 26 | 26 | — | 3 |
| A-03 | | | | | |
| [21] [PB 15] | 64 | 25 | 4 | 4 | 3 |
| A-05 | | | | | |

*C—O bonding may also contribute to the intensity of this band.

TABLE 10-3
Nitrogen Chemistries of PB 15 Samples (% of total N).

| | | | Aromatic |
|---|---|---|---|
| Sample | N—C═N | CN—Cu | Shake-up |
| [—] [PB 15 - untreated] | 79 | 9 | 12 |
| [10] [PB 15] A-02 | 77 | 8 | 15 |
| [14] [PB 15] A-03 | 76 | 16 | 9 |
| [21] [PB 15] A-05 | 87 | 7 | 6 |

TABLE 10-4
Oxygen Chemistries of PB 15 Samples (% of total O).

| | C═O, | |
|---|---|---|
| | COONa, | |
| Sample | Sox | C—O |
| [—] [PB 15 - untreated] | 69 | 31 |
| [10] [PB 15] A-02 | 38 | 62 |
| [14] [PB 15] A-03 | 4 | 96 |
| [21] [PB 15] A-05 | 69 | 31 |

### Tables for PR 122/PV19 Samples

TABLE 11-1
XPS Surface Concentrations of PR 122/PV19 Samples (Atomic %).

| Sample | C | N | O | Na | S | Cl |
|---|---|---|---|---|---|---|
| [—] [PR 122 - untreated] | 84.4 | 8.0 | 7.7 | — | — | — |
| [17] [PR 122] A-06 | 79.0 | 5.0 | 15.6 | 0.3 | — | — |
| [22] [PR 122] A-57 | 82.0 | 6.1 | 10.2 | 1.6 | 0.14 | — |
| [6] [PV 19] S-03 | 80.6 | 5.1 | 12.9 | 0.7 | 0.4 | 0.2 |
| [7 ][PV 19] A-01 | 82.4 | 6.7 | 10.2 | 0.6 | — | — |

TABLE 11-2
Carbon Chemistries of PR 122/PV 19 Samples (% of total C).

| | | | | C═O, | COONa, | Aromatic |
|---|---|---|---|---|---|---|
| Sample | C—C, H | C₂NH ^{#} | C—O | O—C—O | CSO₃Na | Shake-up |
| [-] [PR 122 - untreated] | 66 | 24 | — | 3 | — | 7 |
| [17] [PR 122] A-06 | 55 | 14 | 26 | 2 | — | 3 |
| [22] [PR 122] A-57 | 70 | 21 | — | 1 | 2 | 6 |
| [6] [PV 19] S-03 | 71 | 13 | 6 | 6 | 1 | 3 |
| [7] [PV 19] A-01 | 54 | 18 | 12 | 11 | 2 | 3 |

^{#} C₂NH denotes each of the C atoms bonded in the following group:

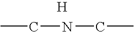

TABLE 11-3
Oxygen Chemistries of PR 122/PV 19 Samples (% of total O).

| | C═O, | | |
|---|---|---|---|
| | COONa, | | Aromatic |
| Sample | SOx | C—O | Shake-up |
| [—] [PR 122 - untreated] | 73 | 15 | 11 |
| [17] [PR 122] A-06 | 21 | 78 | 1 |
| [22] [PR 122] A-57 | 65 | 25 | 10 |
| [6] [PV 19] S-03 | 42 | 55 | 3 |
| [7] [PV 19] A-01 | 43 | 50 | 7 |

### Tables for PY 74 Samples

TABLE 12-1
XPS Surface Concentrations of PY 74 Samples (Atomic %).

| Sample | C | N | O | Na | S |
|---|---|---|---|---|---|
| [—] [PY 74 - | 64.6 | 13.8 | 20.8 | 0.3 | 0.3 |
| untreated] | | | | | |
| [23] [PY 74] A-49 | 69.6 | 9.5 | 19.1 | 1.6 | 0.1 |
| [34] [PY 74] A-34 | 66.8 | 9.7 | 21.5 | 1.0 | 0.3 |

TABLE 12-2
Carbon Chemistries of PY 74 Samples (% of total C).

| | | | | | COONa/ | Aromatic |
|---|---|---|---|---|---|---|
| Sample | C—C,H | C—NH* | C—O | C═O | CSO₃Na | Shake-up |
| [—] [PY | 45 | 17 | 21 | 11 | 1.8 | 4 |
| 74 - | | | | | | |
| untreated] | | | | | | |
| [23] [PY 74] | 72.5 | 8.3 | 12.4 | 5.7 | — | 1.2 |
| A-49 | | | | | | |
| [34] [PY 74] | 63.7 | 9.4 | 18.8 | 6.6 | — | 1.6 |
| A-34 | | | | | | |

*C—O bonding may also contribute to the intensity of this band.

TABLE 12-3
Nitrogen Chemistries of PY 74 Samples (% of total N).

| Sample | C—N | NO₂ | NO₃ |
|---|---|---|---|
| [—] [PY 74 - untreated] | 71 | 9 | 20 |
| [23] [PY 74] A-49 | 76 | 8 | 16 |
| [34] [PY 74] A-34 | 78 | 8 | 14 |

TABLE 12-4
Oxygen Chemistries of PY 74 Samples (% of total O).

| | C═O, | |
|---|---|---|
| Sample | COONa, SOx | C—O, NOₓ |
| [—] [PY 74 - untreated] | 41 | 59 |
| [23] [PY 74] A-49 | 39 | 61 |
| [34] [PY 74] A-34 | 33 | 67 |

Tables for PY 155 samples

TABLE 13-1
XPS Surface Concentrations of PY 155 samples (Atomic %)

| Sample | C | N | O | Na | S |
|---|---|---|---|---|---|
| [—] [PY 155 - untreated] | 67.2 | 10.8 | 22.0 | — | — |
| [5] [PY 155] A-14 | 70.1 | 8.3 | 21.1 | 0.4 | 0.04 |
| [4] [PY 155] S-11 | 68.2 | 9.5 | 21.9 | 0.4 | 0.03 |

TABLE 13-2
Carbon Chemistries of PY 155 samples (% of total C)

| | | | | O—C═O^{#}, | |
|---|---|---|---|---|---|
| | | | | COONa, | Aromatic |
| Sample | C—C,H | C—N* | C═O | SO₃Na | Shake-up |
| [—] [PY 155 - | 57 | 17 | 9 | 14 | 3 |
| untreated] | | | | | |
| [5] [PY 155] A-14 | 60 | 18 | 8 | 12 | 2 |
| [4] [PY 155] S-11 | 57 | 19 | 9 | 12 | 3 |

*C—O bonding may also contribute to the intensity of this band.
^{#}O—C═O is likely the main component of this band, as Na and S concentrations are very low

TABLE 13-3
Oxygen Chemistries of PY 155 samples (% of total O)

| Sample | C═O, COONa, Sulfate | C—O |
|---|---|---|
| [—] [PY 155 - untreated] | 54 | 46 |
| [5] [PY 155] A-14 | 54 | 46 |
| [4] [PY 155] S-11 | 55 | 45 |

The XPS results indicate that the surface modification as disclosed yields a modified carbon black with an increase in surface nitrogen, as an NH/N—C═N group distributed almost equally, in about 0.3 to 2.6 atomic %. It is bonded in NH and N—C═N groups of comparable concentrations. A smaller contribution of NO₃ group is also observed on one sample [8] [Carbon] A-04.

The XPS results indicate that the surface modification as disclosed yields a modified carbon black with a surface oxygen in the atomic ratio of 6.5 to 15.1% wherein ˜43 to 72% of the oxygen is present as C═O, COONa, or SOx group and the balance (˜57 to 28%) as a C—O group. In contrast, the surface oxygen in the untreated carbon black is only about 2.4 atomic % and is distributed as 32% as a C═O, COONa or SOx group and the balance (68%) as a C—O group. The XPS results indicate that the surface modification as disclosed yields a modified carbon black with an increase in surface sodium, as COONa, in about 1.4 to 5.3 atomic %.

The XPS results for untreated carbon blacks, Sensijet SDP 1000 carbon and carbon blacks from Examples 3, 8, 24, 25-31, and 32 is displayed in FIGS. 1-12.

The XPS results indicate that surface modification as disclosed yields a modified Pigment Blue No. 15 with significantly higher surface sodium content (0.8 to 4.2 atomic %) compared to a low concentration of 0.1 atomic % in the untreated pigment. The XPS results for untreated Pigment Blue No. 15 and Pigment Blue No. 15 from Examples 10, 14, and 21 are displayed in FIGS. 13-19.

The XPS results indicate that the surface modification as disclosed yields a modified Pigment Red No. 122 with a surface sodium present at concentrations in the range of 0.3-1.6 atomic % while in comparison the untreated pigment has none. The XPS results for untreated Pigment Red No. 122, Pigment Red No. 122 from Examples 17 and 22, and Pigment Violet 19 from Examples 6 and 7 are displayed in FIGS. 20-25.

The XPS indicate that the surface modification as disclosed yields a modified Pigment Yellow No. 74 with a surface sodium in the atomic ratio of 1.0 to 1.6% which is expected to be present as COONa/CSO₃Na. In contrast, in the untreated pigment the surface sodium is only about 0.3. The XPS results for untreated Pigment Yellow No. 74 and Pigment Yellow No. 74 from Examples 23 and 34 are displayed in FIGS. 26-31. The XPS results for untreated Pigment Yellow No. 155 and for Pigment Yellow No. 155 from Examples 11 and 12 are displayed in FIGS. 32-36.

### Example 38

TABLE 14
Elemental analysis (% C,H,N, & S).

| Sample - [Ex#] | | | | | [S] | | | [H] |
|---|---|---|---|---|---|---|---|---|
| [Pigment Type] | C | H | N | S²⁶ | mmol/g | Na²⁶ | K²⁶ | mmol/g |
| 1 [—] [PB 15 - untreated] | 66.78 | 3.09 | 18.42 | 0.25 | 0.078 | — | — | — |
| 2 [21] [PB 15¹²]A-05 | 62.04 | 3.52 | 14.87 | 0.36 | 0.112 | 1.53 | 0.26 | 0.732 |
| 3 [10] [PB 15²]A-02 | 65.28 | 4.34 | 13.62 | 0.16 | 0.050 | 0.75 | 0.52 | 0.459 |
| 4 [14] [PB 15⁵]A-03 | 62.45 | 4.09 | 14.78 | 0.19 | 0.059 | 0.19 | 1.22 | 0.395 |
| 5 [—] [PR122¹³ - untreated] | 76.79 | 4.72 | 8.16 | — | — | — | — | — |
| 6 [22] [PR 122¹³]A-57 | 71.47 | 4.80 | 6.15 | 0.45 | 0.140 | 1.87 | 0.38 | 0.911 |
| 7 [17] [PR 122⁸]A-06 | 71.95 | 5.50 | 6.57 | 0.11 | 0.034 | 0.31 | 0.24 | 0.196 |
| 8 [6] [PV 19³]S-03 | 71.68 | 4.64 | 7.34 | 0.28 | 0.087 | 0.55 | .42 | 0.347 |
| 9 [7] [PV 19³]A-01 | 71.64 | 4.7 | 7.08 | 0.08 | 0.025 | 0.48 | 0.29 | 0.283 |
| 10 [33] [PV 19³]A-02 | 73.67 | 4.93 | 7.62 | 0.07 | 0.022 | 0.55 | 0.20 | 0.290 |
| 11 [—] [PY 74⁷ - untreated] | 52.98 | 4.47 | 13.53 | 0.31 | 0.097 | — | — | — |
| 12 [23] [PY 74¹⁴]A-49 | 54.11 | 5.04 | 10.91 | 0.21 | 0.065 | 1.54 | 0.34 | 0.757 |
| 13 [34] [PY 74¹⁴]A-34 | 53.04 | 4.68 | 12.55 | 0.26 | 0.081 | 0.41 | 0.07 | 0.196 |
| 14 [—] [PY 155 - untreated] | 56.69 | 4.35 | 11.55 | 0.19 | 0.059 | — | — | — |
| 15 [11] [PY 155⁴]A-14 | 57.53 | 5.05 | 8.88 | 0.11 | 0.034 | 0.74 | 0.47 | 0.442 |
| 16 [12] [PY 155⁴]S-11 | 57.44 | 4.79 | 9.14 | 0.24 | 0.075 | 0.24 | 0.17 | 0.148 |
| 17 [—] [Carbon¹⁵ - untreated] | 91.35 | 1.15 | 0.10 | 0.32 | 0.100 | — | — | — |
| 18 [—] [Carbon^{16 -} oxidized] | 75.12 | 2.03 | — | 0.19 | 0.059 | 2.93 | 0.01 | 1.277 |
| 19 [3] [Carbon¹]A-01 | 86.89 | 1.48 | 0.50 | 0.29 | 0.090 | 0.68 | 0.42 | 0.403 |
| 20 [8] [Carbon¹]A-04 | 77.51 | 2.02 | 1.01 | 0.30 | 0.094 | 1.80 | 0.90 | 1.013 |
| 21 [24] [Carbon¹⁵]A-51 | 79.45 | 1.51 | 0.90 | — | — | 1.59 | 0.65 | 0.858 |
| 22 [25] [Carbon¹⁶]A-21 | 75.46 | 1.97 | 0.94 | — | — | 1.74 | 1.09 | 1.036 |
| 23 [26] [Carbon¹⁶]A-27 | 76.33 | 1.40 | 0.29 | — | — | 2.19 | 1.43 | 1.318 |
| 24 [27] [Carbon¹⁶]A-29 | 75.28 | 1.98 | 0.64 | — | — | 1.98 | 0.60 | 1.015 |
| 25 [28] [Carbon¹⁶]A-31 | 78.59 | 1.33 | 0.12 | — | — | 2.56 | 0.76 | 1.308 |
| 26 [29] [Carbon¹⁶]A-33 | 76.26 | 1.86 | 0.34 | — | — | 2.23 | 1.19 | 1.274 |
| 27 [30] [Carbon¹⁶]A-37 | 74.55 | 1.88 | <0.50 | — | — | 2.50 | 1.30 | 1.420 |
| 28 [31] [Carbon¹⁶]A-43 | 76.44 | 1.27 | 0.89 | — | — | 3.30 | 0.58 | 1.584 |
| 29 [32] [Carbon¹⁵]A-53 | 79.51 | 2.04 | 0.88 | — | — | 1.81 | 0.73 | 0.974 |

²⁶The sulfur, sodium and potassium were calculated @ 100% solids from ICP metal analysis of the original dispersion.

The results of the elemental analysis indicate that the surface modification as disclosed yields a modified Pigment Blue No. 15 with 0.050-0.112 mMoles of 5 and 0.395-0.732 mMoles of active hydrogen per gram of pigment.

The results of the elemental analysis indicate that the surface modification as disclosed yields a modified Pigment Red No. 122 with 0.034-0.140 mMoles of 5 and 0.196-0.911 mMoles of active hydrogen per gram of pigment.

The results of the elemental analysis indicate that the surface modification as disclosed yields a modified Pigment Yellow No. 74 with 0.065-0.081 mMoles of 5 and 0.196-0.757 mMoles of active hydrogen per gram of pigment

The results of the elemental analysis indicate that the surface modification as disclosed yields a modified Pigment Yellow No. 155 with 0.034-0.075 mMoles of 5 and 0.148-0.442 mMoles of active hydrogen per gram of pigment

The results of the elemental analysis indicate that the surface modification as disclosed yields a modified Pigment Violet No. 19 with 0.022-0.087 mMoles of 5 and 0.283-0.347 mMoles of active hydrogen per gram of pigment

The results of the elemental analysis indicate that the surface modification as disclosed yields a modified Carbon Black with 0.403-1.584 mMoles of active hydrogen per gram of pigment.

### Example 39

### Particle Size Measurement

Samples comprising 8-15% solids were prepared by diluting one drop of sample to 15 ml deionized water and loading into a 1 cm disposable cuvette, avoiding air bubbles. Malvern Zetasizer Nano series Model ZEN3600 was then used to measure mean particle size in the sample.

TABLE 15
Particle Size Measurements and Stability data of Pigment Dispersions.

| Example | Pigment | Viscosity | | | Particle Size | | | pH | |
|---|---|---|---|---|---|---|---|---|---|
| [#] | Type | Initial | Week 1 | Week 3 | Initial | Week 1 | Week 3 | Initial | Final |
| 22 | PR122¹³ | 2.20 | 2.19 | 2.21 | 100.3 | 100.9 | 102 | 8.9 | 8.5 |
| 17 | PR122⁸ | 4.27 | 3.57 | 3.62 | 129.4 | 127.5 | 126.2 | 8.1 | 7.0 |
| 6 | PR122¹³ | 2.86 | 2.66 | 2.83 | 137.8 | 134.7 | 137 | 8.2 | 7.9 |
| 14 | PB15⁵ | 5.77 | 3.92 | 3.89 | 131.5 | 129.7 | 133.2 | 8.5 | — |
| 5 | PB15² | 2.80 | 3.09 | 3.11 | 136.3 | 135.1 | 131.9 | 7.7 | 7.8 |
| 10 | PB15² | 9.15 | 6.05 | 5.67 | 102 | 102 | 109 | 8.3 | 8.2 |
| 11 | PY155⁴ | 2.89 | 2.71 | 2.42 | 150 | 152 | 154 | 8.1 | 7.3 |
| 12 | PY155⁴ | 2.90 | 2.90 | 2.80 | 151 | 165 | 178 | 8.2 | 7.4 |
| 23 | PY74¹⁴ | 2.44 | 3.45 | 3.64 | 99 | 113 | 130 | 9.9 | 9.1 |
| 34 | PY74¹⁴ | 1.97 | 2.06 | 2.03 | 125 | 128 | 129 | 8.7 | 8.0 |
| 32 | Carbon¹⁶ | 3.44 | — | 3.69 | 120 | 111 | 116 | 9.0 | 8.6 |

### Example 40

### Redispersion Studies

The following dispersions (Ex #22-24) were dried and redispersed in DI water of pH 7.5 as a powder, as described below.

About 0.5 g of the dry powder was mixed with DI water (pH=7.5), made up to about 80.0 g and sonicated for 5 minutes. A part of the dispersions were then filtered through 0.7 micron GF/F (available from Fisher Scientific) 25 mm diameter syringe filters and the weight of the residue and filtrate, after drying were recorded. The results in Table 16 shows that >93% the modified pigments were effectively redispersed in neutral water. The average particle size (D50, nm) shows that even under these extreme conditions the particles do resist agglomeration.

TABLE 16
Redispersion of modified pigments from Examples 22, 23 and 24.

| | Example #22 | Example #23 | Example #24 |
|---|---|---|---|
| Weight of dried powder (g) | 0.5112 | 0.5065 | 0.5038 |
| Weight of mix in DI water (g) | 80.004 | 80.1351 | 80.0226 |
| Weight of sample filtered | 29.2631 | 29.7757 | 30.3676 |
| Average Particle Size | 135 | 152 | 136 |
| (D₅₀) nm | | | |
| Dry weight of filtrate | 0.1827 | 0.1823 | 0.1780 |
| Dry weight of residue | 0.0014 | 0.0009 | 0.0032 |
| % Redispersed | 97.71 | 96.87 | 93.10 |

### Example 41

The following ink base was made according to the procedure described below and used to make final inks with black dispersions.

TABLE 17
Ink Base I formulation.

| Ingredients | % by Weight |
|---|---|
| Water, deionized | 9.6 |
| 2-Pyrrolidone water blend | 10.0 |
| 1,5-pentanediol | 5.0 |
| PEG 600 Carb. Polyethylene Glycol | 4.0 |
| Nipacide BIT 20 | 0.3 |
| Surfynol 104E solution | 0.1 |
| 1,2-hexanediol | 1.0 |

First, 9.6% by weight of water was added to a clean vessel. A mixing device was then placed inside the vessel to agitate the water and provide mixing while the other ingredients are added. Mixing was achieved by using a magnetic stirring device. Next, 10% by weight of 2-pyrrolidone, 5% by weight of 1,5-pentanediol, 4% by weight of PEG 600, and 1% by weight of 1,2-hexanediol were added to the vessel. These were allowed to dissolve. Then, 0.1% by weight of Surfynol 104E solution and 0.3% by weight of Nipacide BIT 20 were added and allowed to dissolve.

### Example 42

The following inks were made according to the procedure described below using pigment dispersion from Example #24.

TABLE 18
Inks A-C.

| | Ink A | | |
|---|---|---|---|
| | Matt | Ink B | Ink C |
| | Black | Photo Black | Light Grey |
| Water, deionized | 35.58 | 47.38 | 65.3 |
| (g) | | | |
| Dispersion (g) | 34.42 | 22.62 | 4.70 |
| Inkbase (g) | 30.00 | 30.00 | 30.00 |
| Surfynol 465 (g) | 0.17 | 0.17 | 0.17 |
| Surfynol 440 (g) | 0.12 | 0.12 | 0.12 |

A second vessel was prepared by adding calculated % by weight of DI water to the pigment dispersion to the vessel per Table 18. A magnetic stirring device was then placed into the vessel. Next the ink base, followed by surfynol surfactants (Air Products & Chemicals, Inc., Allentown, Pa.), were slowly added to the pigment dispersion in the second vessel. The dispersion was mixed during this process. After all of the diluent has been added, the ink was mixed for about 1 hour, or until it was completely homogenous. After mixing, the ink was filtered using a 1 micron glass filter (available from Whatman, Kent, England).

### Example 43

The following ink base was made according to the procedure described below and used to make final inks with color dispersions.

TABLE 19
Ink Base II formulation.

| Ingredients | % by Weight |
|---|---|
| Water | 12.3 |
| Glycerine | 14 |
| PEG 600 | 2 |
| Butyl Carbitol | 3 |
| TEA | 0.1 |
| Cobratec | 0.3 |
| Xbinx 19G | 0.3 |
| Ethanol | 2 |
| Butanol | 1 |

First, 12.3% by weight of water was added to a clean vessel. A mixing device was then placed inside the vessel to agitate the water and provide mixing while the other ingredients are added. Mixing was achieved by using a magnetic stirring device. Next, 14% by weight of glycerine, 2% by weight of PEG 600, 3% by weight of butyl carbitol, 2% by weight of ethanol, and 1% by weight of butanol were added to the vessel. These were allowed to dissolve. Then, 0.1% by weight of triethanolamine was added and allowed to dissolve. Finally, 0.3% by weight of Cobratec solution and 0.3% by weight of Xbinx 19G were added and allowed to dissolve.

### Example 44

The following inks were made according to the procedure described below.

TABLE 20
Inks D-L.

| | | Ink E | Ink F | Ink G | Ink H |
|---|---|---|---|---|---|
| | Ink D | Dark | Light | Dark | Light |
| | Yellow | Cyan | Cyan | Magenta | Magenta |
| Pigment | Example | Example | Example | Example | Example |
| Dispersion | #23 | #21 | #21 | #22 | #22 |
| from: | | | | | |
| Water, | 41.00 | 38.13 | 48.90 | 35.71 | 55.04 |
| deionized (g) | | | | | |
| Dispersion | 24.00 | 26.87 | 16.10 | 29.29 | 9.96 |
| (g) | | | | | |
| Ink base (g) | 35.00 | 35.00 | 35.00 | 35.00 | 35.00 |
| Surfynol | 0.087 | 0.087 | 0.087 | 0.17 | 0.17 |
| 465 (g) | | | | | |
| Surfynol | 0.058 | 0.058 | 0.058 | 0.12 | 0.12 |
| 440 (g) | | | | | |

A second vessel was prepared by adding the calculated percentage by weight of DI water to the pigment dispersion to the vessel per Table 20. A magnetic stirring device was then placed into the vessel. Next the ink base, followed by surfynol surfactants (Air Products & Chemicals, Inc., Allentown, Pa.), were slowly added to the pigment dispersion in the second vessel. The dispersion was mixed during this process. After all of the diluent has been added, the ink was mixed for about 1 hour, or until it was completely homogenous. After mixing, the ink was filtered using a 1 micron glass filter (available from Whatman, Kent, England).

### Example 45

### Print Performance

### Print Testing of Ink Set Made with Dispersions from Examples 21-24 with Mixed 4-ABA and SMA Attachment

Test pages were printed with an Epson C88+ printer Model B251A and HP Photosmart Plus B9180 printer (known to use pigmented ink sets) using four different commonly used copy papers. The printed pages, identified by ink set and media, were analyzed by the Center for Integrated Manufacturing, Rochester Institute of Technology, Rochester, N.Y. Image Quality was measured with ImageXpert Full Motion System. Optical Density was measured with X-rite 939 Spectrodensitometer. Ozone Exposure was measured using RIT custom ozone chamber and Sutherland Rub test was done with Sutherland rub fixture. Highlighter A is Sanford Yellow Major Accent® and Highlighter B is Avery Dennison Fluorescent Yellow Hi-Liter®. Ozone fading is defined by RIT as follows: “The color change is described by calculating the Delta E 2000 (ÄE00) and reporting according to ASTM D2244-02 Calculation of Color Tolerances and Color Differences from Instrumentally Measured Color Coordinates.” Mottle is determined as follows: “A solid color block is broken into regions of interest (ROI) and the grayness is measured in each region. The average and standard deviation is calculated for the entire solid block. The larger the standard deviation, the more mottle in the sample.”

The print performance characteristics of the color set using ink A, D, E and G printed with Epson C88+ printer is identified below:

TABLE 21

| | Black | Yellow | Cyan | Magenta | Black | Yellow | Cyan | Magenta |
|---|---|---|---|---|---|---|---|---|
| | HP MP-ColorLok | | | | Xerox 4200 | | | |
| Optical | 1.16 | 0.626 | 0.833 | 0.952 | 0.993 | 0.632 | 0.81 | 0.818 |
| Density | | | | | | | | |
| Rub | 0.04 | 0.01 | 0.03 | 0.01 | 0.02 | 0.01 | 0.03 | 0.01 |
| Resistance | | | | | | | | |
| (OD Diff) | | | | | | | | |
| Highlighter A | 0.115 | — | 0.049 | 0.055 | 0.024 | — | 0.002 | 0.017 |
| Resistance | | | | | | | | |
| (OD Diff) | | | | | | | | |
| Highlighter B | 0.039 | — | 0.015 | 0.021 | 0.009 | — | 0.006 | 0.017 |
| Resistance | | | | | | | | |
| (OD Diff) | | | | | | | | |
| Water | 0.015 | 0.007 | 0.015 | 0.022 | 0.005 | 0 | 0.006 | 0.009 |
| resistance (OD | | | | | | | | |
| Diff) | | | | | | | | |
| Ozone Fade | 1.037 | 0.369 | 2.11 | 1.792 | 1.11 | 0.754 | 3.173 | 1.954 |
| Mottle | 2.353 | 1.913 | 2.169 | 2.201 | 1.924 | 1.477 | 1.746 | 1.536 |
| | Office Depot 104 | | | | Hammerill GW | | | |
| Optical Density | 1.068 | 0.674 | 0.873 | 0.898 | 0.963 | 0.655 | 0.833 | 0.826 |
| Rub Resistance | 0.01 | 0.01 | 0.02 | 0.01 | 0.03 | 0.01 | 0.03 | 0.02 |
| (OD Diff) | | | | | | | | |
| Highlighter A | 0.01 | — | 0.012 | 0.019 | 0.049 | — | 0.038 | 0.03 |
| Resistance (OD | | | | | | | | |
| Diff) | | | | | | | | |
| Highlighter B | 0.011 | — | 0.007 | 0.011 | 0.036 | — | 0.022 | 0.021 |
| Resistance (OD | | | | | | | | |
| Diff) | | | | | | | | |
| Water | 0.006 | 0 | 0.001 | 0.005 | 0.006 | 0.003 | 0.004 | 0.009 |
| resistance (OD | | | | | | | | |
| Diff) | | | | | | | | |
| Ozone Fade | 0.884 | 0.53 | 3.507 | 1.827 | 0.881 | 0.482 | 2.518 | 1.732 |
| Mottle | 1.699 | 1.871 | 1.757 | 1.427 | 2.777 | 1.833 | 1.92 | 2.985 |

The print performance characteristics of the color set using ink A, B, C, D, E, F and G printed with HP Photosmart Pro B9180 printer is identified below:

TABLE 22

| | Black | Yellow | Cyan | Magenta | Black | Yellow | Cyan | Magenta |
|---|---|---|---|---|---|---|---|---|
| | HP MP-ColorLok | | | | Xerox 4200 | | | |
| Optical Density | 1.099 | 1.084 | 0.97 | 0.928 | 0.839 | 0.901 | 0.842 | 0.79 |
| Rub Resistance | 0.05 | 0.01 | 0.06 | 0.02 | 0.02 | 0.01 | 0.04 | 0.01 |
| (OD Diff) | | | | | | | | |
| Highlighter A | 0.135 | — | 0.085 | 0.108 | 0 | — | 0.019 | 0 |
| Resistance (OD | | | | | | | | |
| Diff) | | | | | | | | |
| Highlighter B | 0.051 | — | 0.041 | 0.019 | 0.01 | — | 0.021 | 0.002 |
| Resistance (OD | | | | | | | | |
| Diff) | | | | | | | | |
| Water | 0.01 | 0.008 | 0.012 | 0.018 | 0.005 | 0.002 | 0.006 | 0.014 |
| resistance (OD | | | | | | | | |
| Diff) | | | | | | | | |
| Ozone Fade | 1.798 | 1.101 | 2.703 | 2.497 | 1.28 | 1.112 | 2.167 | 1.656 |
| Mottle | 1.595 | 1.141 | 1.768 | 2.85 | 2.064 | 1.681 | 1.336 | 2.071 |
| | Office Depot 104 | | | | Hammerill GW | | | |
| Optical Density | 0.906 | 0.917 | 0.906 | 0.911 | 0.781 | 0.835 | 0.848 | 0.834 |
| Rub Resistance | 0.01 | 0.01 | 0.04 | 0.01 | 0.03 | 0.01 | 0.05 | 0.01 |
| (OD Diff) | | | | | | | | |
| Highlighter A | 0.017 | — | 0.011 | 0.017 | 0.037 | — | 0.032 | 0.023 |
| Resistance (OD | | | | | | | | |
| Diff) | | | | | | | | |
| Highlighter B | 0.005 | — | 0.013 | 0.007 | 0.02 | — | 0.032 | 0.009 |
| Resistance (OD | | | | | | | | |
| Diff) | | | | | | | | |
| Water | 0.004 | 0.002 | 0.003 | 0.011 | 0.007 | 0.003 | 0.004 | 0.011 |
| resistance (OD | | | | | | | | |
| Diff) | | | | | | | | |
| Ozone Fade | 1.063 | 0.983 | 2.693 | 1.68 | 1.398 | 0.839 | 1.94 | 1.417 |
| Mottle | 1.671 | 1.534 | 1.413 | 1.88 | 3.161 | 3.35 | 2.53 | 2.773 |

The print results show that the pigment dispersions produced by the process described in the corresponding experiments produce dispersions suitable to make high quality pigmented inkjet inks. Side by side comparison of these prints with print output from the dispersions made by the process disclosed in U.S. Patent Publication No. US20090050014A1, published Feb. 26, 2009 which is hereby incorporated by reference, shows that these prints are more vibrant and equally durable.

### Example 46

### Wood Stain Application Performance

The following wood stains were prepared and tested at 6% dry pigment loading with a resin solution consisting of 18% Joncryl 95 (available from BASF) and the balance de-ionized water. Waterfastness comparison of drawdowns on Leneta Form 3NT-3 using a wire wound rod #7 (available from Paul N. Gardner Company, Pompano Beach, Fla.) was done with 1″×4″ strips. Half of each strip was dipped in de-ionized water for one minute. The strips were allowed to dry at ambient temperature. The color difference (DE*) was read with Datacolor SF600 PLUS-CT colorimeter. Lower DE* indicates better waterfastness.

TABLE 23
Wood stain comparison.

| Example | Pigment | Dipped area vs. Control | | | | | |
|---|---|---|---|---|---|---|---|
| [#] | Type | DL* | Da* | Db* | DC* | DH* | DE* |
| 24 | Carbon¹⁵ | 0.00 | 0.00 | −0.01 | −0.01 | 0.00 | 0.01 |
| 22 | PR122¹³ | 0.34 | 0.42 | −0.23 | 0.47 | −0.10 | 0.59 |
| 23 | PY74¹⁴ | −0.37 | 0.22 | −1.15 | −1.16 | −0.17 | 1.23 |
| 21 | PB15¹² | −0.27 | 0.25 | −0.09 | −0.02 | 0.27 | 0.38 |

### Example 47

### Coating Performance

The following coating formulations (Masstone) were prepared and tested at 6% dry pigment loading with a resin solution consisting of 25% acrylic vehicle (available from Valspar, Wheeling, Ill.) and the balance de-ionized water. Each Masstone color was mixed with a latex based tint base (available from Sherwin Williams, Cleveland, Ohio) at 1:10 ratio for the tint preparation. The drawdown was prepared on Leneta form 2A using a 6.0 mil wire wound rod. Chemical resistance was measured separately by spotting 10 drops of 10% hydrochloric acid and 10 drops of 10% sodium hydroxide solution on a Masstone drawdown. The degree of chemical resistance is measured by taking the DE* value between the spotted area versus the control area.

TABLE 24
Coating resistance to strong acid (10% Hydrochloric acid).

| Example | Pigment | Spotted area vs Control | | | | | |
|---|---|---|---|---|---|---|---|
| [#] | Type | DL* | Da* | Db* | DC* | DH* | DE* |
| 24 | Carbon¹⁵ | 0.11 | 0.01 | −0.19 | −0.11 | −0.16 | 0.22 |
| 22 | PR122¹³ | −0.43 | −1.67 | −0.76 | −1.79 | −0.41 | 1.88 |
| 23 | PY74¹⁴ | −0.73 | 0.01 | −1.15 | −1.13 | −0.23 | 1.36 |
| 21 | PB15¹² | 0.12 | −0.63 | 1.38 | −1.47 | 0.35 | 1.52 |

TABLE 25
Coating resistance to strong base (10% Sodium hydroxide).

| Exam- | | | | | | | |
|---|---|---|---|---|---|---|---|
| ple | Pigment | Spotted area vs Control | | | | | |
| [#] | Type | DL* | Da* | Db* | DC* | DH* | DE* |
| 24 | Carbon¹⁵ | 1.15 | −0.02 | −0.04 | −0.04 | −0.01 | 1.15 |
| 22 | PR122¹³ | 0.25 | 3.93 | 1.46 | 4.17 | 0.48 | 4.20 |
| 23 | PY74¹⁴ | −22.86 | 20.31 | −35.84 | −22.93 | −34.22 | 47.11 |
| 21 | PB15¹² | −0.95 | −1.53 | 1.49 | −2.11 | −0.34 | 2.34 |

### Example 48

### Color Filter Application Performance

The following color filter formulations were prepared and tested at 6% dry pigment loading adjusted to 75% of the total with de-ionized water and then mixed with a vehicle (25%) consisting of 30% Valspar acrylic vehicle, 30% Joncryl 1972 (available from BASF) and 40% 1-methoxy-2-propanol (Propylene Glycol Monomethyl Ether). Transmission values of the color filter coatings on a transparent olefin polymer substrate using a wire wound rod #7 (Paul N. Gardner Company, Pompano Beach, Fla.) were measured after drying at ambient temperature.

TABLE 26
Transmission Values of Color Filter Coatings.

| | | % Transmittance (nm) | | | | | | | |
|---|---|---|---|---|---|---|---|---|---|
| Example [#] | Pigment Type | 400 | 440 | 480 | 520 | 560 | 600 | 640 | 680 |
| 24 | Carbon¹⁵ | 1.54 | 2.20 | 2.98 | 3.90 | 4.81 | 5.55 | 6.47 | 7.45 |
| 22 | PR122¹³ | 62.05 | 66.44 | 57.49 | 36.60 | 23.90 | 65.52 | 80.51 | 83.28 |
| 23 | PY74¹⁴ | 8.51 | 2.07 | 5.05 | 59.92 | 74.48 | 79.85 | 82.76 | 84.53 |
| 21 | PB15¹² | 43.99 | 73.00 | 81.92 | 71.97 | 22.55 | 3.83 | 3.38 | 6.03 |

### Example 49

### Textile Printing Application Performance

The following printing pastes were prepared and tested at 6% dry pigment loading with Delta Ceramcoat Textile Medium (33%) (available from Delta), Valspar Acrylic Vehicle (5%) and the balance de-ionized water. The drawdowns of the print pastes on a white cotton fabric were prepared using a 6.0 mil wire wound rod. After drying at ambient temperature the prints were heat fixed at 140° C. for 10 minutes in an oven. The fabric was cut into 1″×4″ strips and half of each strip (1″×2″) was immersed in boiling de-ionized water for five minutes. Afterwards, the exposed strips were washed in cold tap water for one minute and allowed to dry at ambient temperature. The washfastness and waterfastness were assessed by measuring the total color difference (DE*) between control and treated fabric.

TABLE 27
Wash and Water Fastness Evaluation.

| Example | Pigment | Washed Fabric vs Control | | | | | |
|---|---|---|---|---|---|---|---|
| [#] | Type | DL* | Da* | Db* | DC* | DH* | DE* |
| 24 | Carbon¹⁵ | −0.04 | 0.02 | 0.21 | 0.21 | 0.06 | 0.22 |
| 22 | PR122¹³ | 1.37 | 10.54 | 2.60 | 10.75 | 1.47 | 10.94 |
| 23 | PY74¹⁴ | 4.20 | −0.57 | 7.77 | 7.66 | 1.41 | 8.85 |
| 21 | PB15¹² | −0.80 | 0.28 | −0.34 | 0.40 | 0.18 | 0.92 |

### Example 50

### Cosmetic Application Performance

The following mascaras (AG8-106A and Glycerine-Water Control) were prepared according to the procedures described below and were tested as visual color strength.

The wax base included Caprylyl Methicone (Dow Corning FZ-3196), Lauryl PEG/PEG-18/18 Methicone (Dow Corning DC 5200) and C30-45 Alkyldimethylsilyl Polypropylsilsesquioxane (Dow Corning DC SW-885 C30 Resin Wax) as emollient, emulsifier and film former respectively.

The pigment dispersions tested included the following:
1. TW 1829 Original formula (Isododecane control) has Isoblack 902 AT20 which is 20% Unipure Black LC 902 in Isododecane. (Isoblack 902 AT20 and Unipure Black LC 902 are available from Sensient Cosmetic Technologies.)
2. Glycerine-Water control (AG8-112A) has Noir Covarine W9793 which is 25%>Unipure Black LC 902 in Water/Glycerin (Noir Covarine W9793 is available from Sensient Cosmetic Technologies.)
3. AG8-106A has Example #28 which is 15.85% SDP Carbon Black dispersion in water.

The procedure followed included the following steps:

(1) Preparation of Phase A by mixing with a propeller blade at 85° C. the ingredients indicated in Table 28 with a propeller blade to get a homogeneous mix.

(2) Add Phase B with mixing.

(3) Prepare Phase C, mix at 60° C. to get a homogeneous mix.

(4) Add Phase C to bulk, emulsify using Turrax homogenizer for 2 minutes.

(5) Add and mix Phase D to bulk and de-aerate.

(6) Pour into an appropriate container at 60° C.

TABLE 28
Formula using dispersions from Example 28 and Glycerine-Water
Control.

| | | Glycerine-Water |
|---|---|---|
| | Example | Pigment |
| | #28 | Dispersion |
| | AG8-106A | AG8-112A |
| | % W/W | % W/W |
| Phase A | | |
| Dow Corning FZ-3196 | 4.00 | 4.00 |
| Dow Corning 5200 Formulation Aid | 6.00 | 6.00 |
| Carnuaba Wax | 2.50 | 2.50 |
| Dow Corning SW-8005 C30 Resin Wax | 4.00 | 4.00 |
| Caprylic/Capric Triglyceride | 3.00 | 3.00 |
| Phase B | | |
| Covabead PMMA 2MUSI (available | 10.00 | 10.00 |
| from Sensient) | | |
| Phase C | | |
| SDP Carbon Dispersion (1.60% dry) | 10.11 | — |
| Noir Covarine W9793 (1.60% dry) | — | 6.40 |
| Preservative (Germaben II) | 1.00 | 1.00 |
| Propylene glycol | 2.50 | 2.50 |
| Pure Water | 11.89 | 15.60 |
| Covacryl P12 (available from Sensient) | 25.00 | 25.00 |
| Phase D | | |
| Isododecane | 20.00 | 20.00 |

The procedure below was followed for the preparation of the isododecane dispersion control:

TABLE 29
Formula for isododecane dispersion control (TW1829).

| | | % W/W |
|---|---|---|
| Phase A | Dow Corning FZ-3196 | 4.00 |
| | Dow Corning 5200 Formulation Aid | 6.00 |
| | Carnauba Wax # 104F | 2.50 |
| | Dow Corning SW-8005 C30 Resin | 4.00 |
| | Wax | |
| | Caprylic/Capric Triglyceride | 3.00 |
| Phase B | Covabead 2 MUSI | 10.00 |
| Phase C | Isoblack 902 AT20 | 8.00 |
| Phase D | Isododecane | 20.00 |
| Phase E | Pure water | 14.00 |
| | Propylene glycol | 2.50 |
| | Preservative | 1.00 |
| Phase F | Covacryl P12 | 25.00 |

(1) Prepare, heat and mix Phase A (same as in Table 28) to 75° C. until homogenous.

(2) Add Phase B (same as in Table 28) to A while mixing using a propeller blade mixer.

(3) Add Phase C (Isoblack 902 AT20, 8.0 g) to Bulk under stifling. Maintain temperature for ≦2 minutes.

(4) Cool Bulk to 65° C. and add Phase D (Isododecane, 20.0 g), mix until homogenous.

(5) Add Phase E (pure water 14.0 g, propylene glycol and preservative 1.0 g) to bulk, emulsify using Turrax homogenizer for 2 minutes.

(6) Add Phase F (Covacryl P12, 25.0 g) to bulk and mix.

(7) Pour into an appropriate container at 55° C.

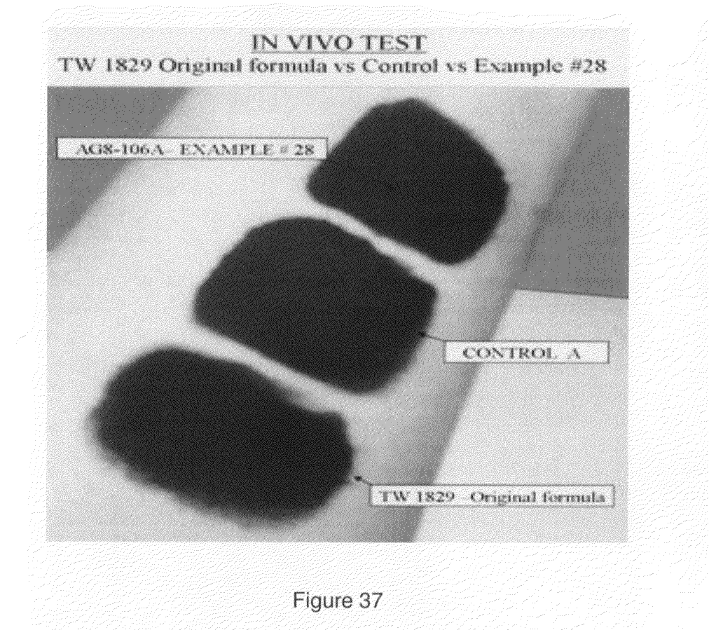

The three mascaras (AG8-106A, Glycerine-Water Control and Isododecane Control (see above for the preparation) were evaluated for in vivo color performance, as shown in FIG. 37. The color of the mascara made with Example # 28 (AG8-106A) is deeper than the other two samples.

What is claimed is:

1. A modified pigment comprising a polymer, wherein the pigment is directly attached to a nitrogen atom, the nitrogen atom being attached directly or indirectly to a group comprising —S-Z, wherein S is a substituted or unsubstituted alkyl group, substituted or unsubstituted aromatic group, or polymer chain having a molecular weight range from about 300 to about 20000, and Z is a hydrogen, carboxyl, sulfonyl, phenolic, phosphoryl, ammonium, trimethylammonium, or tributylammonium group.

2. The modified pigment of claim 1, wherein the nitrogen atom is part of an amino group.

3. The modified pigment of claim 1, wherein S is an aromatic group.

4. The modified pigment of claim 2, wherein the nitrogen atom is part of (a) aminobenzoic acid, (b) aminobenzenesulfonic acid, or (c) aminophenol.

5. The modified pigment of claim 1, wherein the polymer has a molecular weight range from about 300 to about 20000.

6. The modified pigment of claim 1, wherein the polymer is an amine containing polymer.

7. A pigment dispersion comprising the modified pigment according to claim 1.

8. The pigment dispersion of claim 7, wherein the pigment dispersion comprises water.

9. The pigment dispersion of claim 7, wherein the dispersion has a solid content of greater than about 40% w/w.

10. An ink jet ink comprising the modified pigment according to claim 1.

11. The modified pigment of claim 1, wherein the modified pigment is redispersible in an aqueous or non-aqueous medium.

12. The modified pigment of claim 11, wherein the medium is a non-aqueous medium.

13. A modified pigment comprising a polymer, wherein the pigment is attached to an organic group through a carbon atom that is part of a N—C═N bond.

14. The modified pigment of claim 13, wherein the N—C═N bond is part of a triazine.

15. The modified pigment of claim 14, wherein the triazine is substituted with a group comprising S-Z, wherein S is a substituted or unsubstituted alkyl group, substituted or unsubstituted aromatic group, or polymer chain having a molecular weight range from about 300 to about 20000, and Z is a hydrogen, carboxyl, sulfonyl, phenolic, phosphoryl, ammonium, trimethylammonium, or tributylammonium group.

16. The modified pigment of claim 15, wherein the triazine is substituted with the group comprising S-Z and a second group comprising S-Z, the group comprising S-Z and the second group comprising S-Z being the same or different.

17. The modified pigment of claim 14, wherein the triazine is a 1,3,5 triazine.

18. The modified pigment of claim 14, wherein the triazine is substituted with the polymer.

19. A cosmetic formulation comprising a pigment covalently bonded to an organic group.

20. The cosmetic formulation of claim 19, wherein the pigment has attached at least one group comprising N—S, a triazine substituted with at least one group comprising N—S, and a combination thereof, wherein N is a nucleophilic group and S is an organic group.

21. The cosmetic formulation of claim 19, wherein the pigment has attached a polymer.

22. The cosmetic formulation of claim 19, wherein the cosmetic formulation comprises a pigment dispersion.

23. A method of modifying a pigment dispersion, the method comprising:
reacting a substituted triazine with a pigment dispersion.

24. The method of claim 23, wherein reacting the triazine with the pigment dispersion produces a modified pigment dispersion having a modified pigment, and wherein the pigment dispersion comprises a polymer prior to reaction with the triazine, resulting in the modified pigment having a polymer on a surface thereof.

25. The method of claim 23, further comprising reacting a polymer with the triazine and the pigment dispersion.

26. The method of claim 25, wherein the polymer is directly attached to the surface of the pigment.

27. The method of claim 25, wherein the triazine is substituted with at least one polymer.

28. The method of claim 23, wherein the substituted triazine is formed by reacting cyanuric chloride with a secondary compound or a mixture of secondary compounds to displace at least one reactive chlorine to form the substituted triazine.

29. The method of claim 28, wherein about three equivalents of the secondary compound or mixture of secondary compounds are reacted with the cyanuric chloride to displace all reactive chlorines.

30. The method of claim 23, wherein the triazine is substituted with at least one group comprising N—S, wherein N is a nucleophilic group and S is an organic group.

31. The method of claim 30, wherein the group comprising N—S further comprises Z to form a group comprising N—S-Z, and Z is a hydrogen, carboxyl, sulfonyl, phenolic, phosphoryl, ammonium, trimethylammonium, or tributylammonium group.

32. The method of claim 23, wherein the pigment dispersion comprises water.

33. A method of modifying a pigment, the method comprising:
reacting cyanuric chloride with a secondary compound or a mixture of secondary compounds to displace at least one reactive chlorine to form a substituted triazine; and
attaching the substituted triazine and at least one polymer to a surface of a pigment.

34. The method of claim 33, wherein the pigment is dispersed in a medium.

35. The method of claim 33, wherein the polymer attaches to the surface of the pigment through the substituted triazine.

36. The method of claim 33, wherein the polymer attaches directly to the surface of the pigment.

37. A method of modifying a pigment, the method comprising:
displacing at least one reactive chorine of a cyanuric chloride with at least one polymer to form a substituted triazine; and
reacting the substituted triazine with a pigment dispersed in a medium.

38. The method of claim 37, wherein the triazine is further substituted with at least one group comprising N—S, wherein N is a nucleophilic group and S is an organic group.

39. The method of claim 38, wherein the group comprising N—S further comprises Z to form a group comprising N—S-Z, wherein N is an amine, an imine, a pyridine, or a thiol group; S is substituted or unsubstituted alkyls, aryls, or polymer chains having a molecular weight range from about 300 to about 20000; and Z is a hydrogen, carboxyl, sulfonyl, phenolic, phosphoryl, ammonium, trimethylammonium, or tributylammonium group.

40. A method of modifying a pigment, the method comprising:
attaching at least one polymer to a pigment in a pigment dispersion to form a modified pigment, the modified pigment having directly attached a nitrogen atom, and the nitrogen atom being directly or indirectly attached to an organic group.

41. The method of claim 40, wherein the polymer attaches directly to a surface of the pigment.

42. The method of claim 40, wherein the organic group comprises at least one group comprising —S-Z, wherein S is a substituted or unsubstituted alkyl group, substituted or unsubstituted aromatic group, or polymer chain having a molecular weight range from about 300 to about 20000, and Z is a hydrogen, carboxyl, sulfonyl, phenolic, phosphoryl, ammonium, trimethylammonium, or tributylammonium group.

43. The method of claim 40, wherein the nitrogen atom is part of (a) aminobenzoic acid, (b) aminobenzenesulfonic acid, or (c) aminophenol.
